(19) United States
(12) Patent Application Publication
(10) Pub. No.: US 2023/0397494 A1
(43) Pub. Date: Dec. 7, 2023

(54) ORGANIC COMPOUND, CARRIER-TRANSPORT MATERIAL, HOST MATERIAL, LIGHT-EMITTING DEVICE, LIGHT-EMITTING APPARATUS, ELECTRONIC DEVICE, AND LIGHTING DEVICE

(71) Applicant: SEMICONDUCTOR ENERGY LABORATORY CO., LTD., Atsugi-shi, Kanagawa-ken (JP)

(72) Inventors: Hiroshi KADOMA, Sagamihara-shi, Kanagawa (JP); Nobuharu OHSAWA, Zama, Kanagawa (JP); Satoshi SEO, Sagamihara, Kanagawa (JP); Harue OSAKA, Atsugi-shi, Kanagawa (JP)

(21) Appl. No.: 18/250,572

(22) PCT Filed: Oct. 19, 2021
(86) PCT No.: PCT/IB2021/059590

§ 371 Date: Apr. 26, 2023

(30) Foreign Application Priority Data
Oct. 30, 2020 (JP) 2020-182681

(51) Int. Cl. H10K 85/60; C07D 471/06; C09K 11/06

(52) U.S. Cl. CPC H10K 85/6572; C07D 471/06; C09K 11/06; H10K 85/6576; H10K 85/636; H10K 50/166

(57) ABSTRACT

A novel organic compound suitable for a host material of a light-emitting device, particularly a host material of a phosphorescent device is provided. An organic compound in which any of a carbazolyl group, a dibenzothiophenyl group, a dibenzofuranyl group, and a diphenylamino group is bonded to the 3-position of imidazophenanthridine through an arylene group, or an organic compound in which a diphenylamino group is bonded to the 3-position of triazolophenanthridine through an arylene group is provided.

### TECHNICAL FIELD

One embodiment of the present invention relates to an organic compound, a light-emitting element, a light-emitting device, a display module, a lighting module, a display apparatus, alight-emitting apparatus, an electronic device, and alighting device. Note that one embodiment of the present invention is not limited to the above technical field. The technical field of one embodiment of the invention disclosed in this specification relates to an object, a method, or a manufacturing method. One embodiment of the present invention relates to a process, a machine, manufacture, or a composition of matter. Specifically, examples of the technical field of one embodiment of the present invention disclosed in this specification include a semiconductor device, a display apparatus, a liquid crystal display apparatus, a light-emitting apparatus, a lighting device, a power storage device, a memory device, an imaging device, a driving method thereof, and a manufacturing method thereof.

### BACKGROUND ART

Light-emitting devices (organic EL devices) including organic compounds and utilizing electroluminescence (EL) have been put to more practical use. In the basic structure of such light-emitting devices, an organic compound layer containing a light-emitting material (an EL layer) is sandwiched between a pair of electrodes. Carriers are injected by application of a voltage to the device, and recombination energy of the carriers is used, whereby light emission can be obtained from the light-emitting material.

Such light-emitting devices are of self-light-emitting type and thus have advantages over liquid crystal, such as high visibility and no need for backlight when used for pixels of a display, and are suitable as flat panel display elements. Displays including such light-emitting devices are also highly advantageous in that they can be thin and lightweight. Moreover, such light-emitting devices also have a feature that the response speed is extremely fast.

Since light-emitting layers of such light-emitting devices can be successively formed two-dimensionally, planar light emission can be achieved. This feature is difficult to realize with point light sources typified by incandescent lamps and LEDs or linear light sources typified by fluorescent lamps; thus, such light-emitting devices also have a great potential as planar light sources typified by lighting.

Displays or lighting devices including light-emitting devices are suitable for a variety of electronic devices as described above, and research and development of light-emitting devices has progressed for more favorable characteristics. For example, Patent Document 1 discloses a structure in which an imidazophenanthridine derivative is used as a host material of a phosphorescent device, and Patent Document 2 discloses a structure in which a triazolophenanthridine derivative is used as a host material of a phosphorescent device.

### REFERENCES

### Patent Documents

[Patent Document 1] Japanese Published Patent Application No. 2014-033195
[Patent Document 2] Japanese Published Patent Application No. 2017-175128

### SUMMARY OF THE INVENTION

### Problems to be Solved by the Invention

An object of one embodiment of the present invention is to provide a novel organic compound. An object of another embodiment of the present invention is to provide a novel material for a carrier-transport layer. An object of another embodiment of the present invention is to provide a novel host material. An object of another embodiment of the present invention is to provide a host material for a phosphorescent device. An object of another embodiment of the present invention is to provide a novel organic compound with which a light-emitting device having high emission efficiency can be fabricated. An object of another embodiment of the present invention is to provide an organic compound with which a light-emitting device having a long lifetime can be provided.

An object of another embodiment of the present invention is to provide a light-emitting device with high emission efficiency. An object of another embodiment of the present invention is to provide a light-emitting device, a light-emitting apparatus, an electronic device, and a display apparatus each having low power consumption. An object of another embodiment of the present invention is to provide a light-emitting device, a light-emitting apparatus, an electronic device, and a display apparatus each having high reliability.

Note that the description of these objects does not preclude the existence of other objects. One embodiment of the present invention does not need to achieve all these objects. Note that other objects will be apparent from the description of the specification, the drawings, the claims, and the like, and other objects can be derived from the description of the specification, the drawings, the claims, and the like.

It is only necessary that any one of the above-described objects be achieved in the present invention.

### Means for Solving the Problems

In order to achieve the above objects, an organic compound in which any of a carbazolyl group, a dibenzothiophenyl group, a dibenzofuranyl group, and a diarylamino group is bonded to the 3-position of imidazophenanthridine through an arylene group, or an organic compound in which a diarylamino group is bonded to the 3-position of triazolophenanthridine through an arylene group is provided in one embodiment of the present invention.

That is, one embodiment of the present invention is an organic compound represented by General Formula (G1) below.

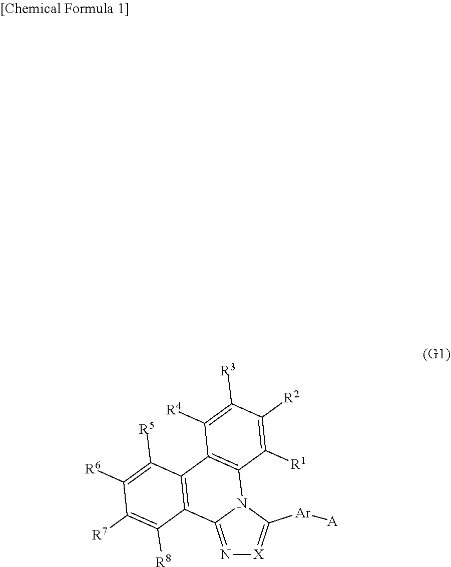

Note that in General Formula (G1) above, X represents nitrogen or substituted or unsubstituted carbon, and Ar represents a substituted or unsubstituted arylene group having 6 to 12 carbon atoms. Furthermore, R¹ to R⁸ each independently represent any of hydrogen, an alkyl group having 1 to 6 carbon atoms, a cyclic alkyl group having 3 to 7 carbon atoms, and a substituted or unsubstituted aryl group having 6 to 13 carbon atoms. Furthermore, A represents a substituted or unsubstituted diarylamino group when X represents nitrogen, and A represents any of a substituted or unsubstituted diarylamino group, a substituted or unsubstituted carbazolyl group, a substituted or unsubstituted dibenzothiophenyl group, and a substituted or unsubstituted dibenzofuranyl group when X represents carbon.

Alternatively, another embodiment of the present invention is the organic compound in the above structure, in which R¹ to R⁸ each represent hydrogen.

Alternatively, one embodiment of the present invention is an organic compound represented by General Formula (G2) below.

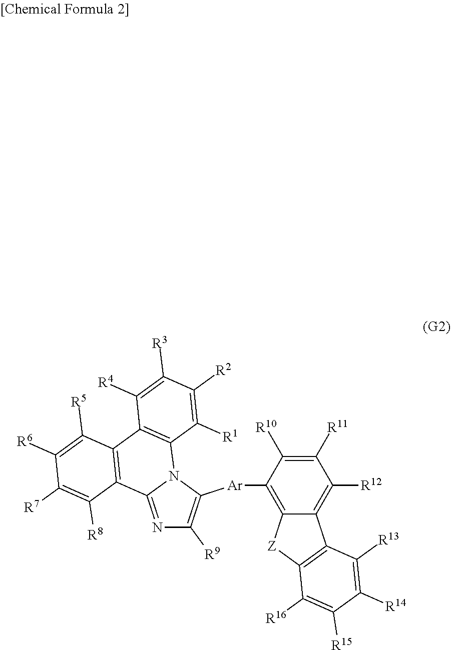

Note that in General Formula (G2) above, Z represents oxygen or sulfur, and Ar represents a substituted or unsubstituted arylene group having 6 to 12 carbon atoms. Furthermore, R¹ to R¹⁶ each independently represent any of hydrogen, an alkyl group having 1 to 6 carbon atoms, a cyclic alkyl group having 3 to 7 carbon atoms, and a substituted or unsubstituted aryl group having 6 to 13 carbon atoms.

Alternatively, another embodiment of the present invention is the organic compound in the above structure, in which R¹ to R¹⁶ each represent hydrogen.

Alternatively, another embodiment of the present invention is an organic compound represented by General Formula (G3) below.

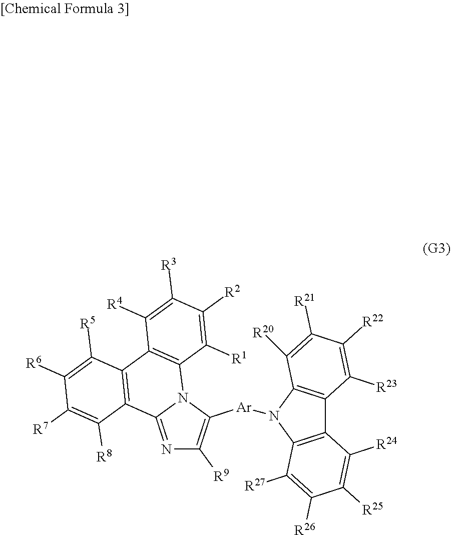

Note that in General Formula (G3) above, Ar represents a substituted or unsubstituted arylene group having 6 to 12 carbon atoms. Furthermore, R¹ to R⁹ and R²⁰ to R²⁷ each independently represent any of hydrogen, an alkyl group having 1 to 6 carbon atoms, a cyclic alkyl group having 3 to 7 carbon atoms, and a substituted or unsubstituted aryl group having 6 to 13 carbon atoms.

Alternatively, another embodiment of the present invention is the organic compound in the above structure, in which R¹ to R⁹ and R²⁰ to R²⁷ each represent hydrogen.

Alternatively, another embodiment of the present invention is the organic compound in the above structure, in which Ar is represented by any of Structural Formulae (Ar-1) to (Ar-3) below.

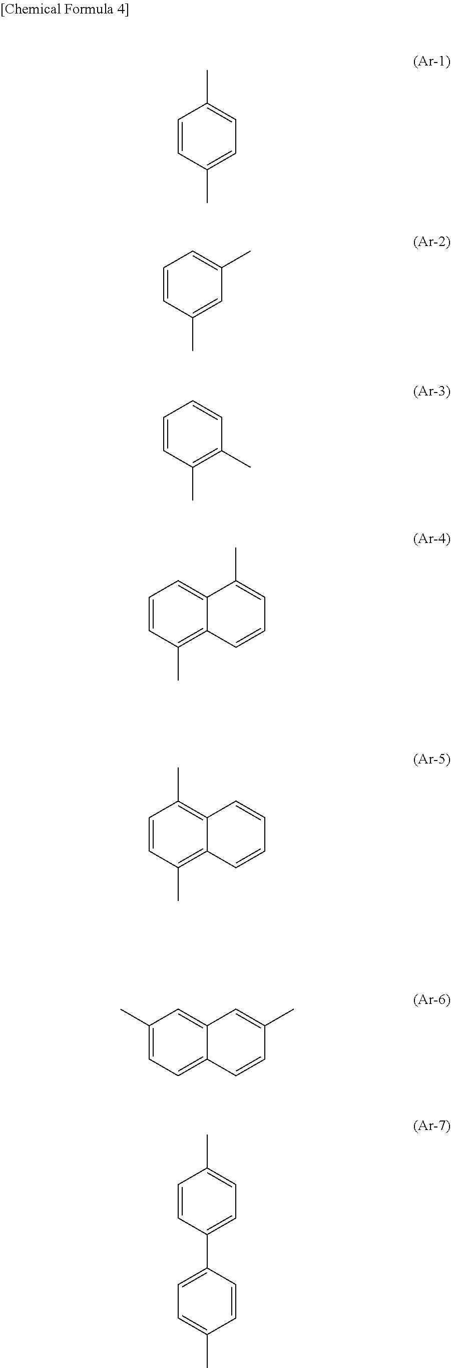

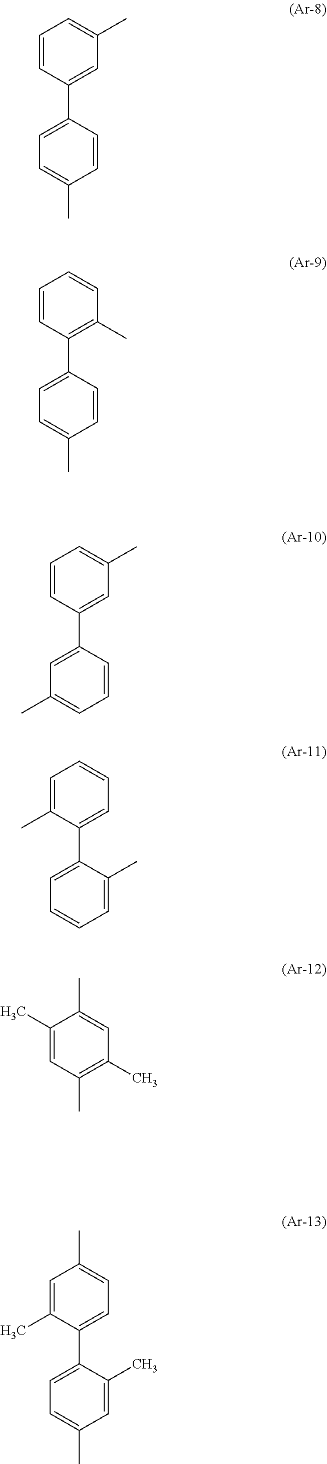

Alternatively, another embodiment of the present invention is the organic compound in the above structure, in which Ar is a group represented by any of Structural Formulae (Ar-1), (Ar-2), (Ar-7), (Ar-8), and (Ar-10) above.

Alternatively, another embodiment of the present invention is the organic compound in the above structure, in which the Ar is a group represented by Structural Formulae (Ar-1) above.

Alternatively, another embodiment of the present invention is an organic compound represented by Structural Formula (100) below.

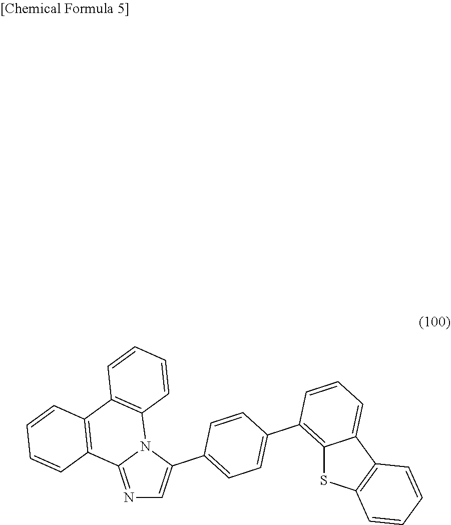 [SMILES: c1ccc2c(c1)sc1c(-c3ccc(-c4cnc5c6ccccc6c6ccccc6n45)cc3)cccc12]

Alternatively, another embodiment of the present invention is an organic compound represented by Structural Formula (135) below.

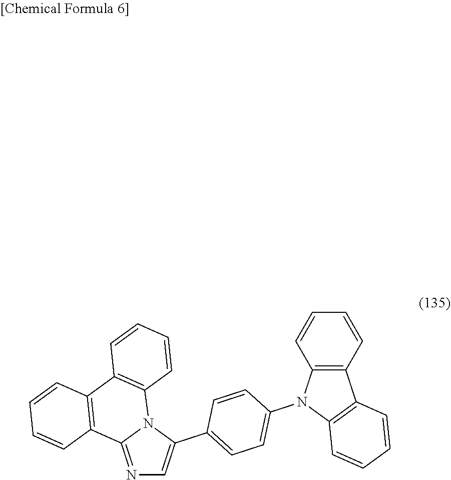 [SMILES: c1ccc2c(c1)c1ccccc1n1c(-c3ccc(-n4c5ccccc5c5ccccc54)cc3)cnc21]

Alternatively, another embodiment of the present invention is a material for a carrier-transport layer of a light-emitting device containing any of the above organic compounds.

Alternatively, another embodiment of the present invention is a host material of a light-emitting device containing any of the above organic compounds.

Alternatively, another embodiment of the present invention is a light-emitting device including an anode, a cathode, and an EL layer positioned between the anode and the cathode. The EL layer includes a light-emitting layer and the light-emitting layer contains a light-emitting material and the above organic compound.

Alternatively, another embodiment of the present invention is an electronic device including the above light-emitting device, and a sensor, an operation button, a speaker, or a microphone.

Alternatively, another embodiment of the present invention is a light-emitting apparatus including the above light-emitting device and a transistor or a substrate.

Alternatively, another embodiment of the present invention is a lighting device including the above light-emitting device and a housing.

Note that the light-emitting apparatus in this specification includes, in its category, an image display device that uses a light-emitting device. The light-emitting apparatus may also include, in its category, a module in which a light-emitting device is provided with a connector such as an anisotropic conductive film or a TCP (Tape Carrier Package), a module in which a printed wiring board is provided at the end of a TCP, and a module in which an IC (integrated circuit) is directly mounted on a light-emitting device by a COG (Chip On Glass) method. Furthermore, a lighting device may include the light-emitting apparatus.

### Effect of the Invention

One embodiment of the present invention can provide a novel organic compound. Another embodiment of the present invention can provide a novel material for a carrier-transport layer. Another embodiment of the present invention can provide a novel host material. Another embodiment of the present invention can provide a host material that can be suitably used for a phosphorescent device. Another embodiment of the present invention can provide a novel organic compound with which a light-emitting device having high emission efficiency can be fabricated. Another embodiment of the present invention can provide an organic compound with which a light-emitting device having a long lifetime can be provided.

Another embodiment of the present invention can provide a light-emitting device with high emission efficiency. Another embodiment of the present invention can provide a light-emitting device, a light-emitting apparatus, an electronic device, and a display apparatus each having low power consumption. Another embodiment of the present invention can provide a light-emitting device, a light-emitting apparatus, an electronic device, and a display apparatus each having high reliability.

Note that the description of these effects does not preclude the existence of other effects. One embodiment of the present invention does not need to have all these effects. Note that other effects will be apparent from the description of the specification, the drawings, the claims, and the like, and other effects can be derived from the description of the specification, the drawings, the claims, and the like.

### BRIEF DESCRIPTION OF THE DRAWINGS

FIG. 1A, FIG. 1B, and FIG. 1C are schematic diagrams of light-emitting devices.

FIG. 2A and FIG. 2B are conceptual views of an active matrix light-emitting apparatus.

FIG. 3A and FIG. 3B are conceptual views of an active matrix light-emitting apparatus.

FIG. 4 is a conceptual view of an active matrix light-emitting apparatus.

FIG. 5A and FIG. 5B are conceptual views of a passive matrix light-emitting apparatus.

FIG. 6A and FIG. 6B are diagrams illustrating a lighting device.

FIG. 7A, FIG. 7B1, FIG. 7B2, and FIG. 7C are diagrams illustrating electronic devices.

FIG. 8A, FIG. 8B, and FIG. 8C are diagrams illustrating electronic devices.

FIG. 9 is a diagram illustrating a lighting device.

FIG. 10 is a diagram illustrating a lighting device.

FIG. 11 is a diagram illustrating in-vehicle display devices and lighting devices.

FIG. 12A and FIG. 12B are diagrams illustrating an electronic device.

FIG. 13A, FIG. 13B, and FIG. 13C are diagrams illustrating an electronic device.

FIG. 14A and FIG. 14B are ¹H NMR charts of DBTPIPt-II.

FIG. 15 shows an absorption spectrum and an emission spectrum of DBTPIPt-II in a toluene solution.

FIG. 16 shows an absorption spectrum and an emission spectrum of DBTPIPt-II in a thin film state.

FIG. 17A and FIG. 17B are ¹H NMR charts of CzPIPt.

FIG. 18 shows an absorption spectrum and an emission spectrum of CzPIPt in a toluene solution.

FIG. 19 shows an absorption spectrum and an emission spectrum of CzPIPt in a thin film state.

FIG. 20A and FIG. 20B are ¹H NMR charts of mDPhATPt.

FIG. 21 shows an absorption spectrum and an emission spectrum of mDPhATPt in a toluene solution.

FIG. 22 shows an absorption spectrum and an emission spectrum of mDPhATPt in a thin film state.

FIG. 23 shows luminance-current density characteristics of a light-emitting device 1, a light-emitting device 2, a comparative light-emitting device 1, and a comparative light-emitting device 2.

FIG. 24 shows current efficiency-luminance characteristics of the light-emitting device 1, the light-emitting device 2, the comparative light-emitting device 1, and the comparative light-emitting device 2.

FIG. 25 shows luminance-voltage characteristics of the light-emitting device 1, the light-emitting device 2, the comparative light-emitting device 1, and the comparative light-emitting device 2.

FIG. 26 shows current-voltage characteristics of the light-emitting device 1, the light-emitting device 2, the comparative light-emitting device 1, and the comparative light-emitting device 2.

FIG. 27 shows external quantum efficiency-luminance characteristics of the light-emitting device 1, the light-emitting device 2, the comparative light-emitting device 1, and the comparative light-emitting device 2.

FIG. 28 shows emission spectra of the light-emitting device 1, the light-emitting device 2, the comparative light-emitting device 1, and the comparative light-emitting device 2.

FIG. 29 shows normalized luminance-time change characteristics of the light-emitting device 1, the light-emitting device 2, the comparative light-emitting device 1, and the comparative light-emitting device 2.

FIG. 30 shows luminance-current density characteristics of a light-emitting device 3 and a comparative light-emitting device 3.

FIG. 31 shows current efficiency-luminance characteristics of the light-emitting device 3 and the comparative light-emitting device 3.

FIG. 32 shows luminance-voltage characteristics of the light-emitting device 3 and the comparative light-emitting device 3.

FIG. 33 shows current-voltage characteristics of the light-emitting device 3 and the comparative light-emitting device 3.

FIG. 34 shows external quantum efficiency-luminance characteristics of the light-emitting device 3 and the comparative light-emitting device 3.

FIG. 35 shows luminance-power efficiency characteristics of the light-emitting device 3 and the comparative light-emitting device 3.

FIG. 36 shows emission spectra of the light-emitting device 3 and the comparative light-emitting device 3.

FIG. 37 shows luminance-current density characteristics of a light-emitting device 4 and a comparative light-emitting device 4.

FIG. 38 shows the current efficiency-luminance characteristics of the light-emitting device 4 and the comparative light-emitting device 4.

FIG. 39 shows luminance-voltage characteristics of the light-emitting device 4 and the comparative light-emitting device 4.

FIG. 40 shows current-voltage characteristics of the light-emitting device 4 and the comparative light-emitting device 4.

FIG. 41 shows external quantum efficiency-luminance characteristics of the light-emitting device 4 and the comparative light-emitting device 4.

FIG. 42 shows luminance-power efficiency characteristics of the light-emitting device 4 and the comparative light-emitting device 4.

FIG. 43 shows emission spectra of the light-emitting device 4 and the comparative light-emitting device 4.

FIG. 44 shows luminance-current density characteristics of a light-emitting device 5 and a comparative light-emitting device 5.

FIG. 45 shows the current efficiency-luminance characteristics of the light-emitting device 5 and the comparative light-emitting device 5.

FIG. 46 shows luminance-voltage characteristics of the light-emitting device 5 and the comparative light-emitting device 5.

FIG. 47 shows current-voltage characteristics of the light-emitting device 5 and the comparative light-emitting device 5.

FIG. 48 shows external quantum efficiency-luminance characteristics of the light-emitting device 5 and the comparative light-emitting device 5.

FIG. 49 is shows luminance-power efficiency characteristics of the light-emitting device 5 and the comparative light-emitting device 5.

FIG. 50 shows emission spectra of the light-emitting device 5 and the comparative light-emitting device 5.

### MODE FOR CARRYING OUT THE INVENTION

Embodiments of the present invention will be described in detail below with reference to the drawings. Note that the present invention is not limited to the following description, and it will be readily appreciated by those skilled in the art that modes and details of the present invention can be modified in various ways without departing from the spirit and scope of the present invention. Therefore, the present invention should not be construed as being limited to the description in the following embodiments.

### Embodiment 1

In this embodiment, an organic compound of one embodiment of the present invention will be described. An organic compound of one embodiment of the present invention is an organic compound represented by General Formula (G1) below.

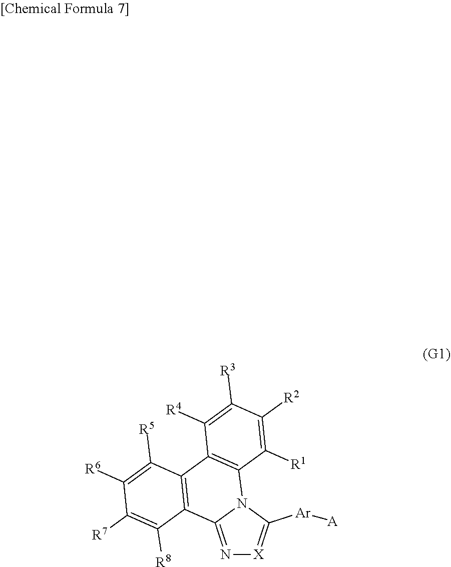

Note that in General Formula (G1) above, X represents nitrogen or substituted or unsubstituted carbon. In the case where X is carbon having a substituent, the substituent is any of an alkyl group having 1 to 6 carbon atoms, a cyclic alkyl group having 3 to 7 carbon atoms, and a substituted or unsubstituted aryl group having 6 to 13 carbon atoms.

In addition, Ar represents a substituted or unsubstituted arylene group having 6 to 12 carbon atoms.

Furthermore, R¹ and R⁸ each independently represent any of hydrogen, an alkyl group having 1 to 6 carbon atoms, a cyclic alkyl group having 3 to 7 carbon atoms, and a substituted or unsubstituted aryl group having 6 to 13 carbon atoms.

Moreover, A represents a substituted or unsubstituted diarylamino group when X represents nitrogen, and represents any of a substituted or unsubstituted diarylamino group, a substituted or unsubstituted carbazolyl group, a substituted or unsubstituted dibenzothiophenyl group, and a substituted or unsubstituted dibenzofuranyl group when X represents carbon.

Note that an aryl group of the above diarylamino group is a substituted or unsubstituted aryl group having 6 to 13 carbon atoms; specifically, a phenyl group, a naphthyl group, a biphenyldiyl group, and a fluorenyl group can each be independently given.

In the organic compound represented by General Formula (G1) above, when R¹ to R⁸ each represent hydrogen, the synthesis is easy and a raw material is easily prepared, so that such organic compound is advantageous in cost.

Note that in the organic compound represented by General Formula (G1) above, it is preferable that X represent carbon and A represent a dibenzothiophenyl group or a dibenzofuranyl group. It is particularly preferable that a dibenzothiophenyl group or a dibenzofuranyl group be bonded to the 4-position of Ar, and an organic compound represented by General Formula (G2) below is preferable.

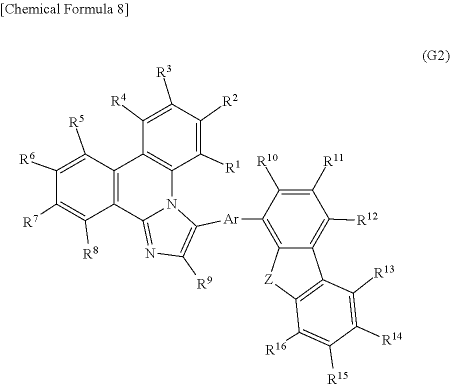

In General Formula (G2) above, Z represents oxygen or sulfur.

In addition, Ar represents a substituted or unsubstituted arylene group having 6 to 12 carbon atoms.

Furthermore, R¹ and R¹⁶ each independently represent any of hydrogen, an alkyl group having 1 to 6 carbon atoms, a cyclic alkyl group having 3 to 7 carbon atoms, and a substituted or unsubstituted aryl group having 6 to 13 carbon atoms.

In the organic compound represented by General Formula (G2) above, when R¹ to R¹⁶ each represent hydrogen, the synthesis is easy and a raw material is easily prepared, so that such organic compound is advantageous in cost.

Furthermore, in the organic compound represented by General Formula (G1) above, it is preferable that X represent carbon and A represent a carbazolyl group because in that case, a favorable hole-transport property is obtained. In particular, a carbazolyl group is preferably bonded to the 9-position of nitrogen Ar, i.e., an organic compound represented by General Formula (G3) below is preferable.

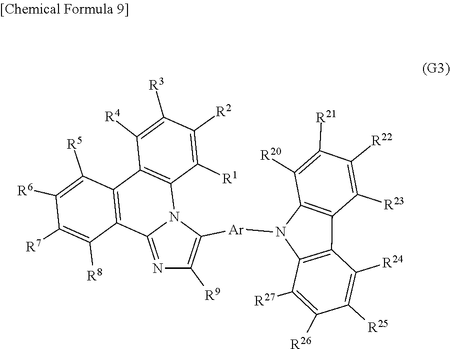

In General Formula (G3) above, Ar represents a substituted or unsubstituted arylene group having 6 to 12 carbon atoms.

Furthermore, R¹ to R⁹ and R²⁰ to R²⁷ each independently represent any of hydrogen, an alkyl group having 1 to 6 carbon atoms, a cyclic alkyl group having 3 to 7 carbon atoms, and a substituted or unsubstituted aryl group having 6 to 13 carbon atoms.

In the organic compound represented by General Formula (G3) above, when R¹ to R⁹ and R²⁰ to R²⁷ each represent hydrogen, the synthesis is easy and a raw material is easily prepared, so that such organic compound is advantageous in cost.

In any of the organic compounds represented by General Formulae (G1) to (G3) above, examples of an arylene group having 6 to 12 carbon atoms represented as Ar include a phenylene group, a naphthylene group, and a biphenyldiyl group; in particular, an organic compound having a group represented by any of Structural Formulae (Ar-1) to (Ar-13) below is preferable. It is particularly preferable that Ar be the group represented by any of Structural Formulae (Ar-1), (Ar-2), (Ar-7), (Ar-8), and (Ar-10) because in that case, a synthesis yield is high and a raw material is inexpensive; in particular, Ar is preferably the group represented by Structural Formula (Ar-1) below.

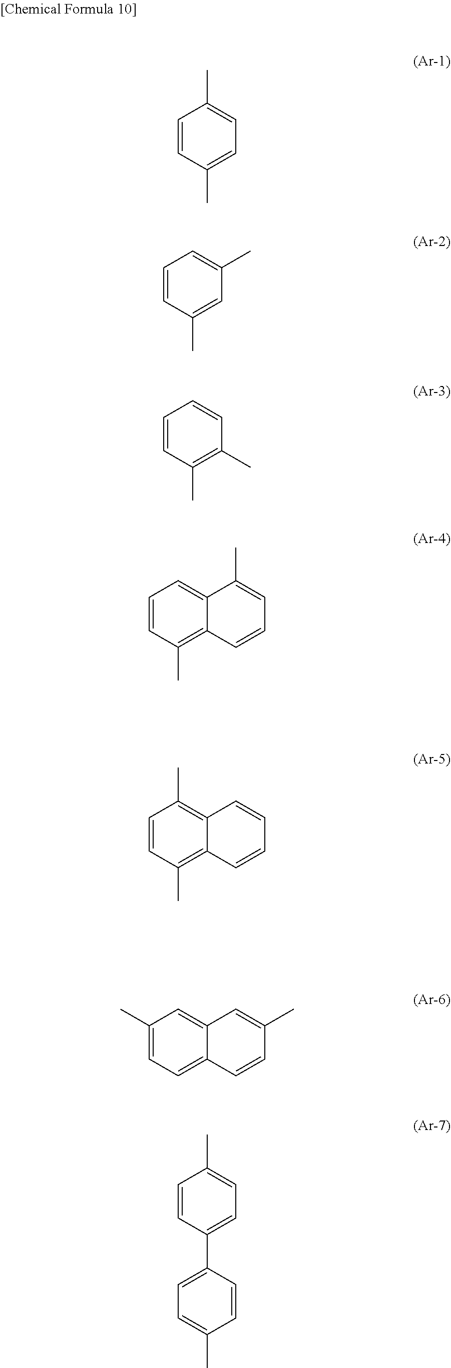

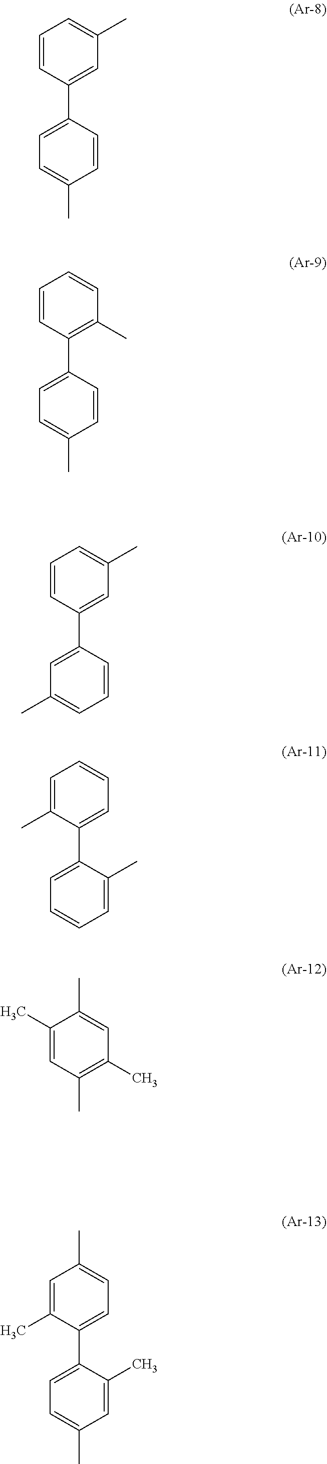

Note that in this specification, when the expression “substituted or unsubstituted” is used in the description of a group and a skeleton, as a substituent that the group and the skeleton can have, an alkyl group having 1 to 6 carbon atoms, a cyclic alkyl group having 3 to 7 carbon atoms, and an aryl group having 6 to 13 carbon atoms can be given. In these substituents, adjacent substituents may form a ring.

In this specification, specific examples of the alkyl group having 1 to 6 carbon atoms include a methyl group, an ethyl group, a propyl group, an isopropyl group, a butyl group, a tert-butyl group, a pentyl group, and a hexyl group. Examples of the cyclic alkyl group having 3 to 7 carbon atoms include a cyclopropyl group, a cyclobutyl group, a cyclopentyl group, a cyclohexyl group, a 1-methylcyclohexyl group, a 2,6-dimethylcyclohexyl group, a cycloheptyl group, and a cyclooctyl group. Examples of the aryl group having 6 to 13 carbon atoms include a phenyl group, a naphthyl group, a biphenyl group, and a fluorenyl group. As the alkyl group having 1 to 6 carbon atoms, the cyclic alkyl group having 3 to 7 carbon atoms, and the aryl group having 6 to 13 carbon atoms, groups represented by Structural Formulae (R-2) to (R-32) below are preferable.

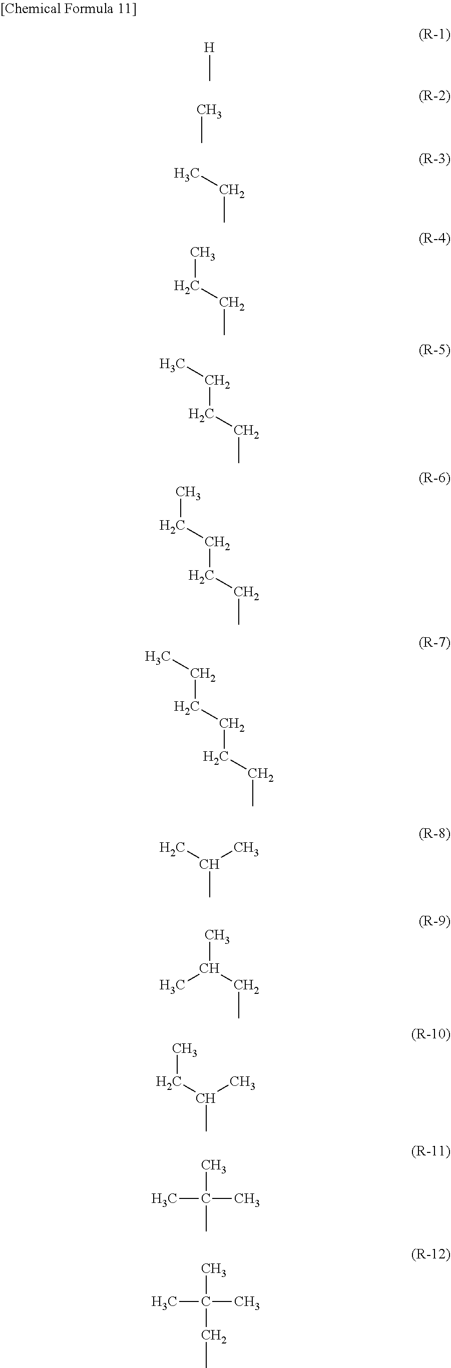

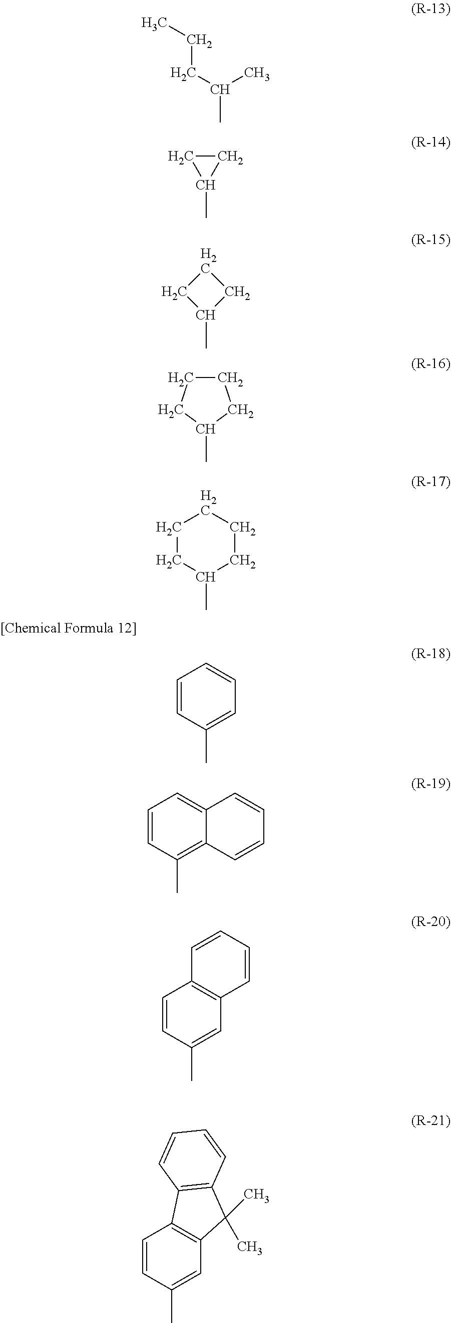

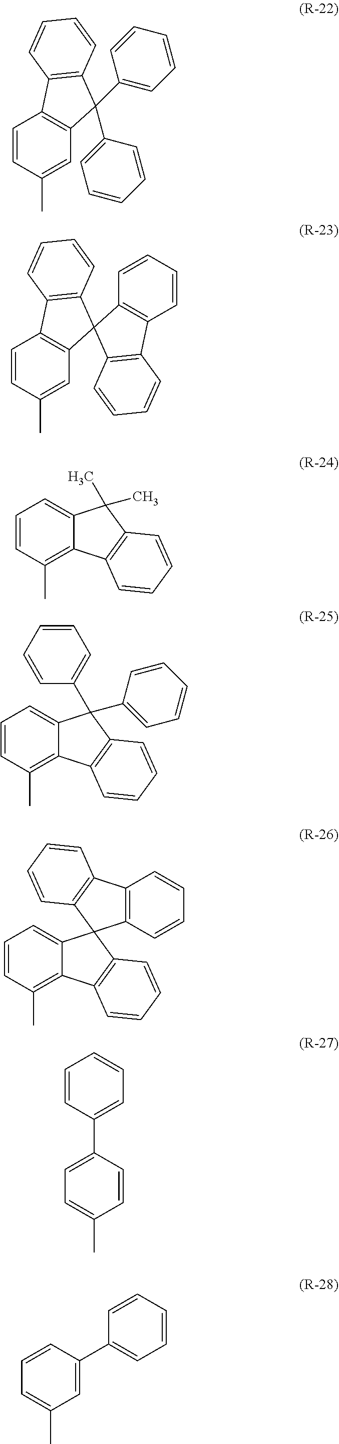

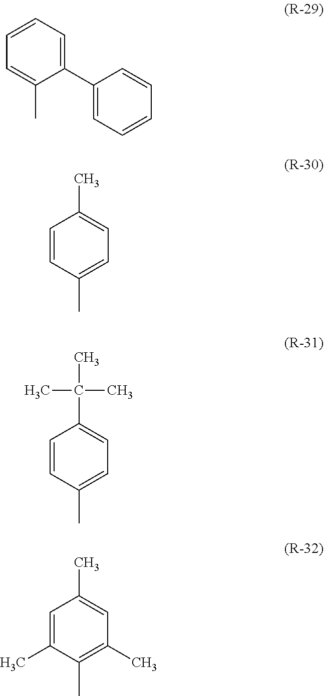

When the organic compound of one embodiment of the present invention having the above structure is used for a light-emitting device, a light-emitting device with high emission efficiency can be provided. A light-emitting device with a long lifetime can also be provided. Furthermore, the organic compound of one embodiment of the present invention can be suitably used as a material for a carrier-transport layer or a host material.

Specific examples of the organic compounds with the above structures are shown below.

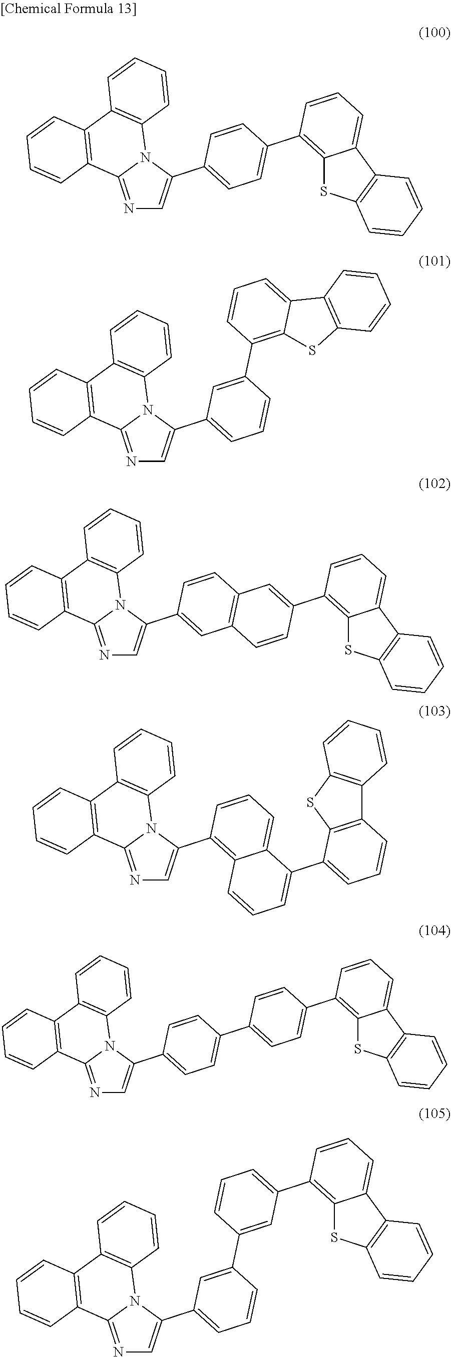

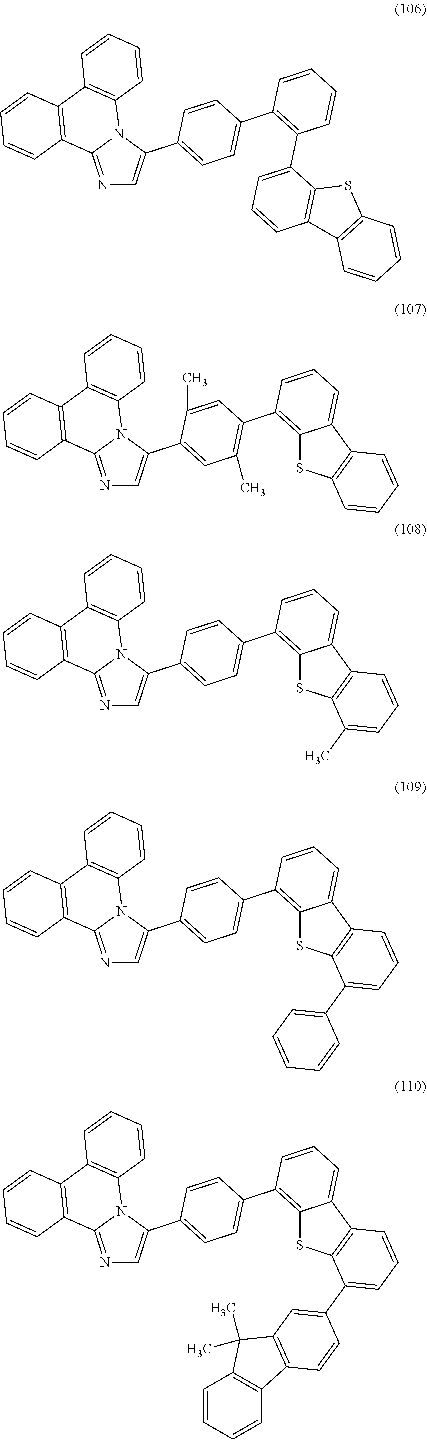 [SMILES: c1ccc(-c2cccc3c2sc2ccccc23)c(-c2ccc(-c3cnc4c5ccccc5c5ccccc5n34)cc2)c1 ; Cc1cc(-c2cnc3c4ccccc4c4ccccc4n23)c(C)cc1-c1cccc2c1sc1ccccc12 ; Cc1cccc2c1sc1c(-c3ccc(-c4cnc5c6ccccc6c6ccccc6n45)cc3)cccc12 ; c1ccc(-c2cccc3c2sc2c(-c4ccc(-c5cnc6c7ccccc7c7ccccc7n56)cc4)cccc23)cc1 ; CC1(C)c2ccccc2-c2ccc(-c3cccc4c3sc3c(-c5ccc(-c6cnc7c8ccccc8c8ccccc8n67)cc5)cccc34)cc21]

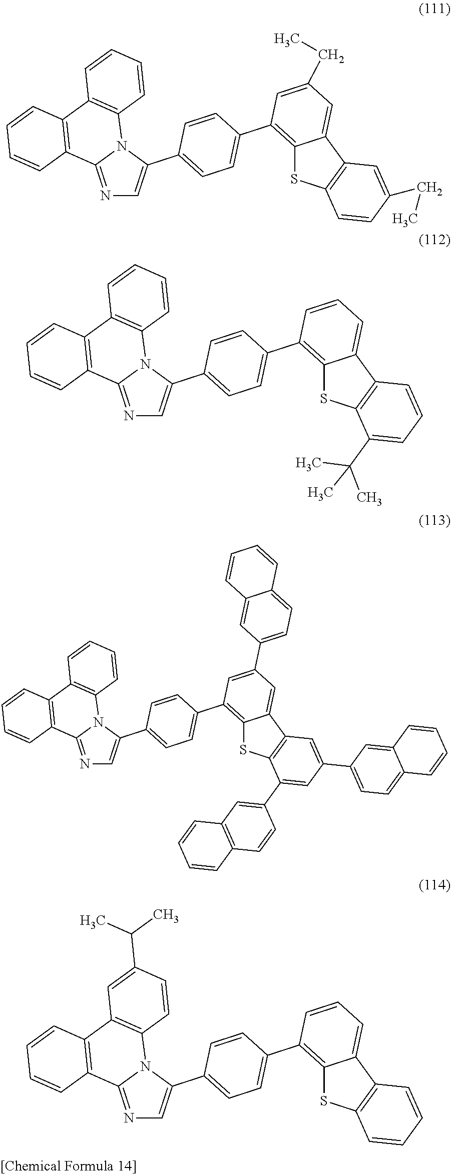 [SMILES: CCc1ccc2sc3c(-c4ccc(-c5cnc6c7ccccc7c7ccccc7n56)cc4)cc(CC)cc3c2c1 ; CC(C)(C)c1cccc2c1sc1c(-c3ccc(-c4cnc5c6ccccc6c6ccccc6n45)cc3)cccc12 ; c1ccc2cc(-c3cc(-c4ccc(-c5cnc6c7ccccc7c7ccccc7n56)cc4)c4sc5c(-c6ccc7ccccc7c6)cc(-c6ccc7ccccc7c6)cc5c4c3)ccc2c1 ; CC(C)c1ccc2c(c1)c1ccccc1c1ncc(-c3ccc(-c4cccc5c4sc4ccccc45)cc3)n21]

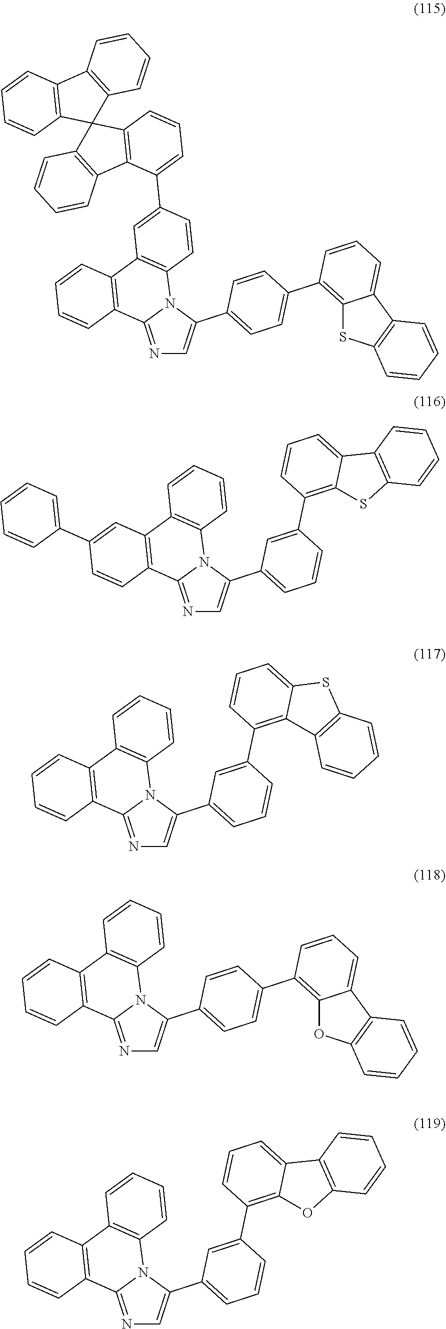 [SMILES: c1ccc2c(c1)-c1ccccc1C21c2ccccc2-c2c(-c3ccc4c(c3)c3ccccc3c3ncc(-c5ccc(-c6cccc7c6sc6ccccc67)cc5)n43)cccc21 ; c1ccc(-c2ccc3c(c2)c2ccccc2n2c(-c4cccc(-c5cccc6c5sc5ccccc56)c4)cnc32)cc1 ; c1cc(-c2cccc3sc4ccccc4c23)cc(-c2cnc3c4ccccc4c4ccccc4n23)c1 ; c1ccc2c(c1)oc1c(-c3ccc(-c4cnc5c6ccccc6c6ccccc6n45)cc3)cccc12 ; c1cc(-c2cccc3c2oc2ccccc23)cc(-c2cnc3c4ccccc4c4ccccc4n23)c1]

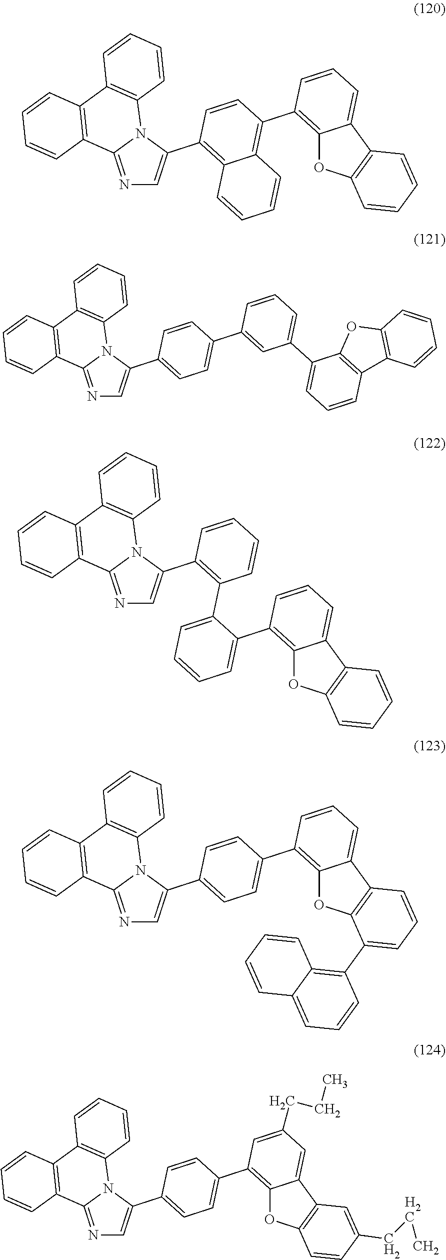 [SMILES: c1ccc2c(c1)oc1c(-c3ccc(-c4cnc5c6ccccc6c6ccccc6n45)c4ccccc34)cccc12 ; c1cc(-c2ccc(-c3cnc4c5ccccc5c5ccccc5n34)cc2)cc(-c2cccc3c2oc2ccccc23)c1 ; c1ccc(-c2cccc3c2oc2ccccc23)c(-c2ccccc2-c2cnc3c4ccccc4c4ccccc4n23)c1 ; c1ccc2c(-c3cccc4c3oc3c(-c5ccc(-c6cnc7c8ccccc8c8ccccc8n67)cc5)cccc34)cccc2c1 ; CCCc1ccc2oc3c(-c4ccc(-c5cnc6c7ccccc7c7ccccc7n56)cc4)cc(CCC)cc3c2c1]

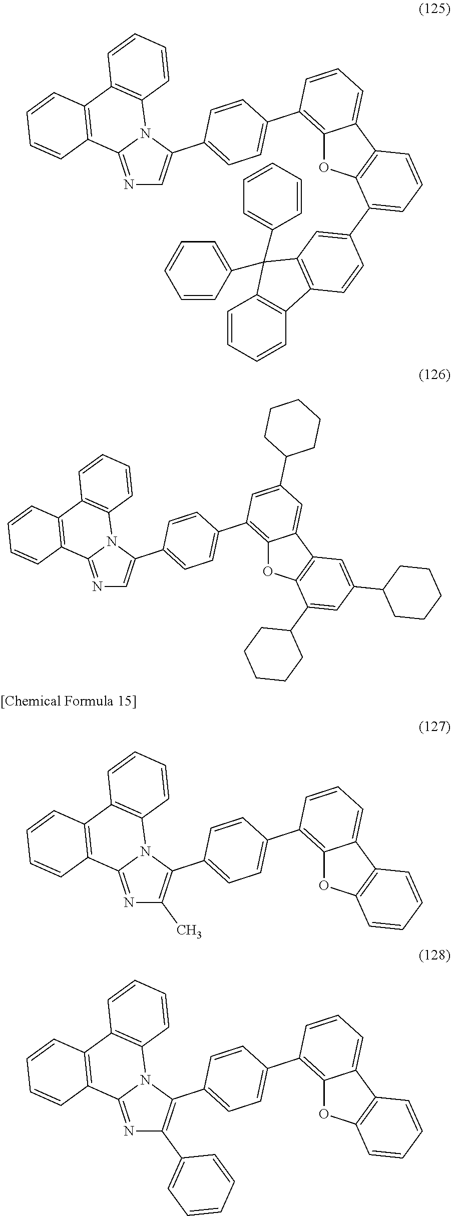 [SMILES: c1ccc(C2(c3ccccc3)c3ccccc3-c3ccc(-c4cccc5c4oc4c(-c6ccc(-c7cnc8c9ccccc9c9ccccc9n78)cc6)cccc45)cc32)cc1 ; c1ccc2c(c1)c1ccccc1n1c(-c3ccc(-c4cc(C5CCCCC5)cc5c4oc4c(C6CCCCC6)cc(C6CCCCC6)cc45)cc3)cnc21 ; Cc1nc2c3ccccc3c3ccccc3n2c1-c1ccc(-c2cccc3c2oc2ccccc23)cc1 ; c1ccc(-c2nc3c4ccccc4c4ccccc4n3c2-c2ccc(-c3cccc4c3oc3ccccc34)cc2)cc1]

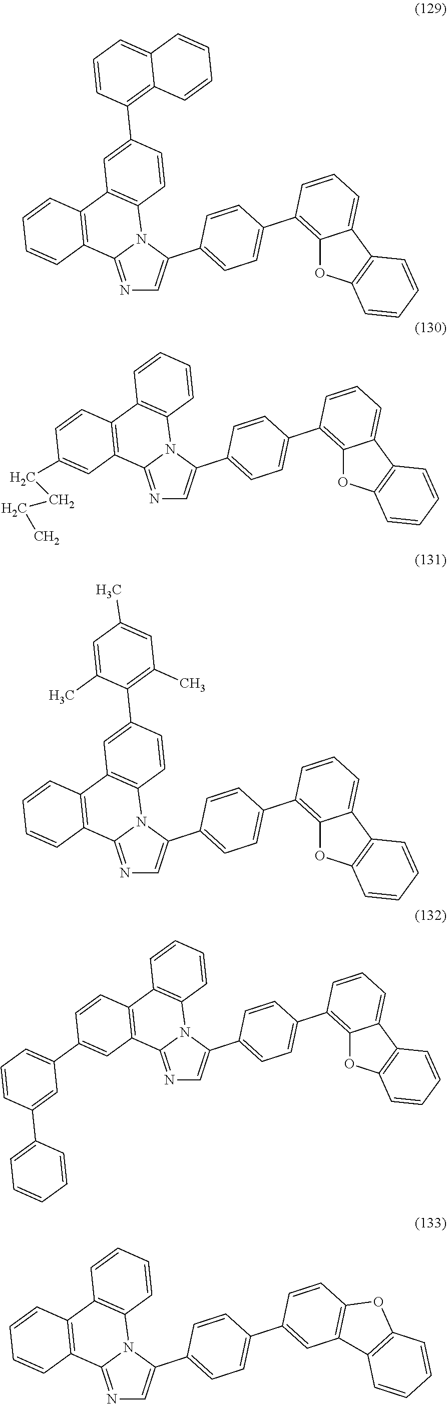 [SMILES: c1ccc2c(-c3ccc4c(c3)c3ccccc3c3ncc(-c5ccc(-c6cccc7c6oc6ccccc67)cc5)n43)cccc2c1 ; CCCCc1ccc2c3ccccc3n3c(-c4ccc(-c5cccc6c5oc5ccccc56)cc4)cnc3c2c1 ; Cc1cc(C)c(-c2ccc3c(c2)c2ccccc2c2ncc(-c4ccc(-c5cccc6c5oc5ccccc56)cc4)n32)c(C)c1 ; c1ccc(-c2cccc(-c3ccc4c5ccccc5n5c(-c6ccc(-c7cccc8c7oc7ccccc78)cc6)cnc5c4c3)c2)cc1 ; c1ccc2c(c1)oc1ccc(-c3ccc(-c4cnc5c6ccccc6c6ccccc6n45)cc3)cc12]

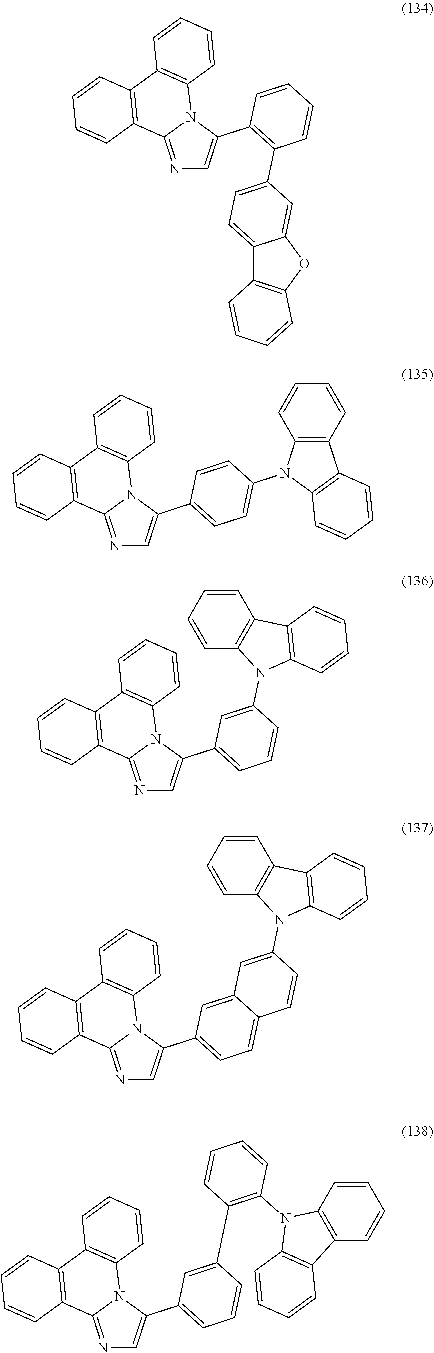 [SMILES: c1ccc(-c2cnc3c4ccccc4c4ccccc4n23)c(-c2ccc3c(c2)oc2ccccc23)c1 ; c1ccc2c(c1)c1ccccc1n1c(-c3ccc(-n4c5ccccc5c5ccccc54)cc3)cnc21 ; c1cc(-c2cnc3c4ccccc4c4ccccc4n23)cc(-n2c3ccccc3c3ccccc32)c1 ; c1ccc2c(c1)c1ccccc1n1c(-c3ccc4ccc(-n5c6ccccc6c6ccccc65)cc4c3)cnc21 ; c1cc(-c2ccccc2-n2c3ccccc3c3ccccc32)cc(-c2cnc3c4ccccc4c4ccccc4n23)c1]

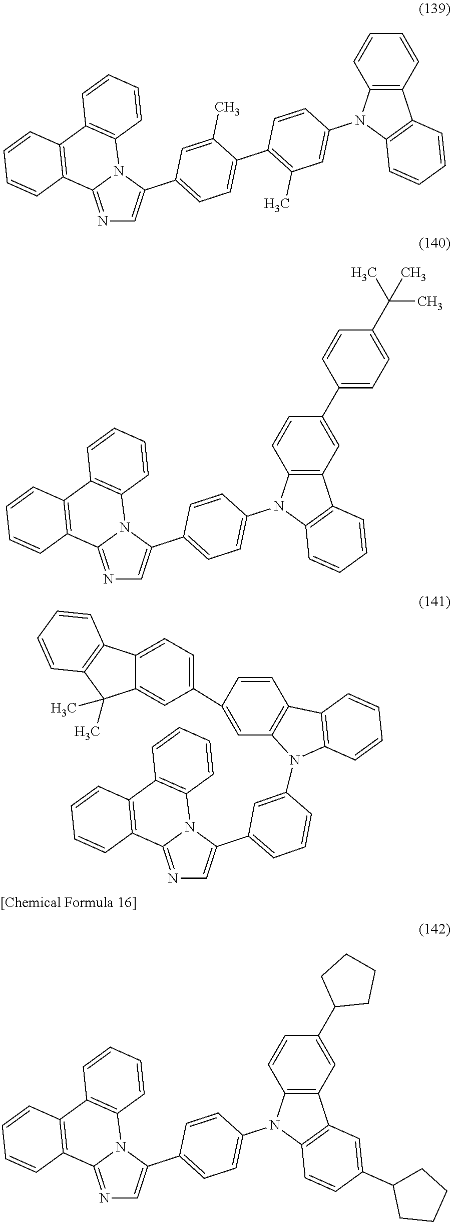

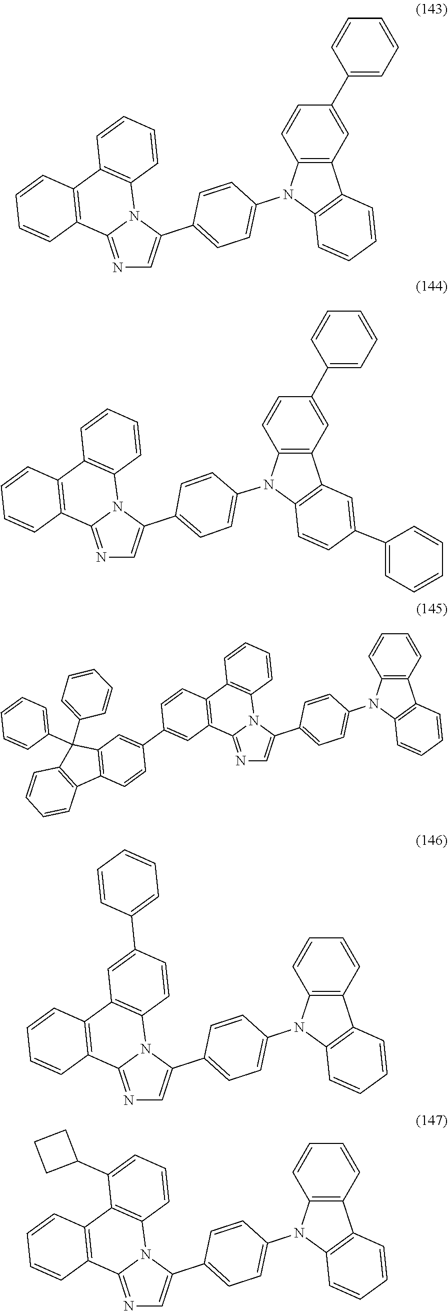

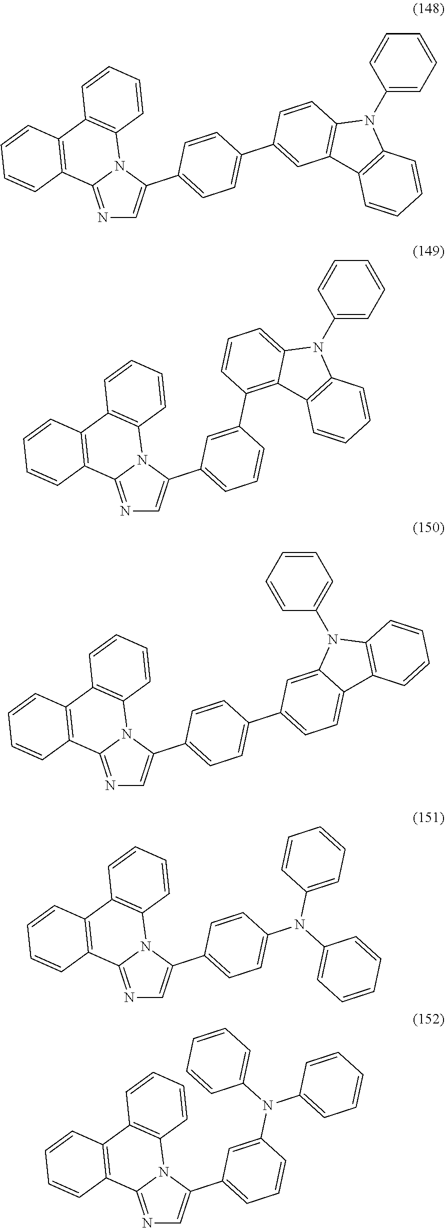 [SMILES: c1ccc(-n2c3ccccc3c3cc(-c4ccc(-c5cnc6c7ccccc7c7ccccc7n56)cc4)ccc32)cc1 ; c1ccc(-n2c3ccccc3c3c(-c4cccc(-c5cnc6c7ccccc7c7ccccc7n56)c4)cccc32)cc1 ; c1ccc(-n2c3ccccc3c3ccc(-c4ccc(-c5cnc6c7ccccc7c7ccccc7n56)cc4)cc32)cc1 ; c1ccc(N(c2ccccc2)c2ccc(-c3cnc4c5ccccc5c5ccccc5n34)cc2)cc1 ; c1ccc(N(c2ccccc2)c2cccc(-c3cnc4c5ccccc5c5ccccc5n34)c2)cc1]

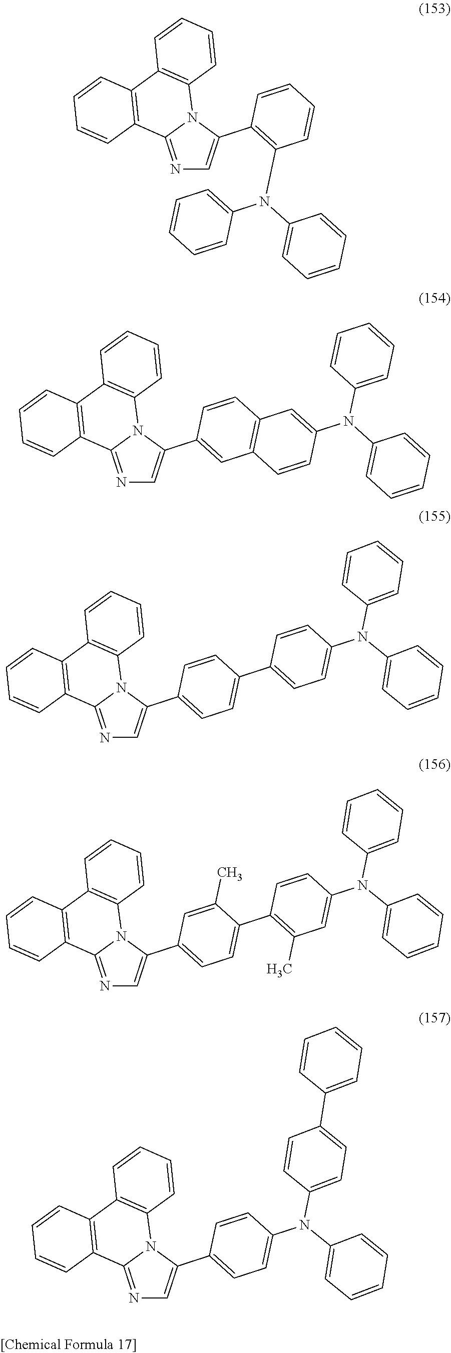

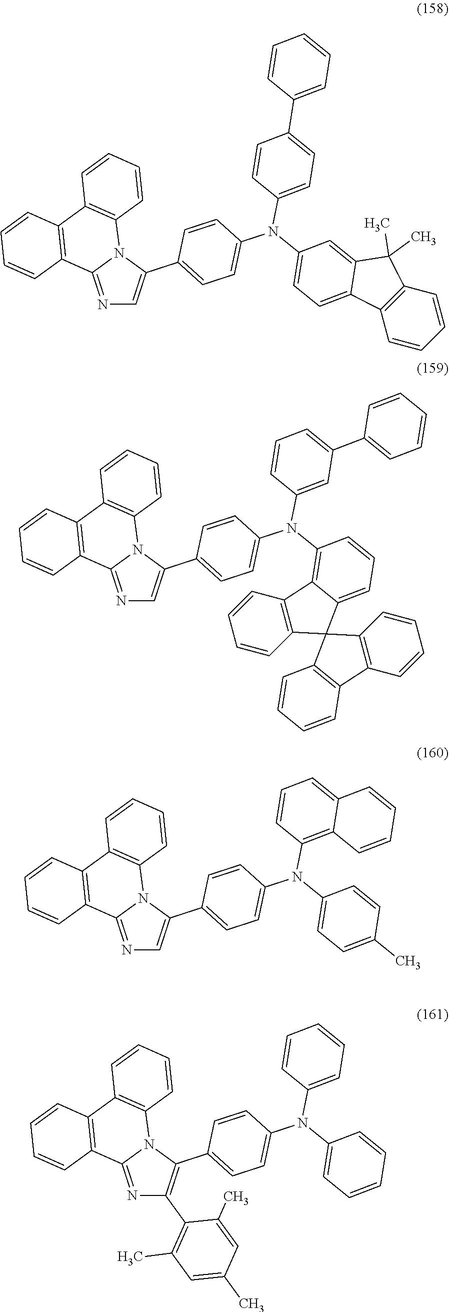 [SMILES: CC1(C)c2ccccc2-c2ccc(N(c3ccc(-c4ccccc4)cc3)c3ccc(-c4cnc5c6ccccc6c6ccccc6n45)cc3)cc21 ; c1ccc(-c2cccc(N(c3ccc(-c4cnc5c6ccccc6c6ccccc6n45)cc3)c3cccc4c3-c3ccccc3C43c4ccccc4-c4ccccc43)c2)cc1 ; Cc1ccc(N(c2ccc(-c3cnc4c5ccccc5c5ccccc5n34)cc2)c2cccc3ccccc23)cc1 ; Cc1cc(C)c(-c2nc3c4ccccc4c4ccccc4n3c2-c2ccc(N(c3ccccc3)c3ccccc3)cc2)c(C)c1]

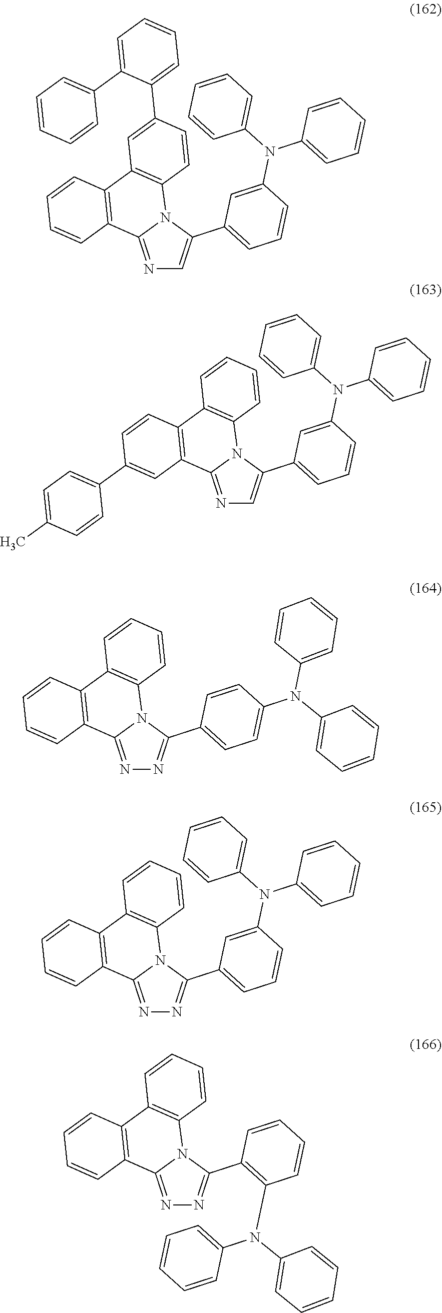

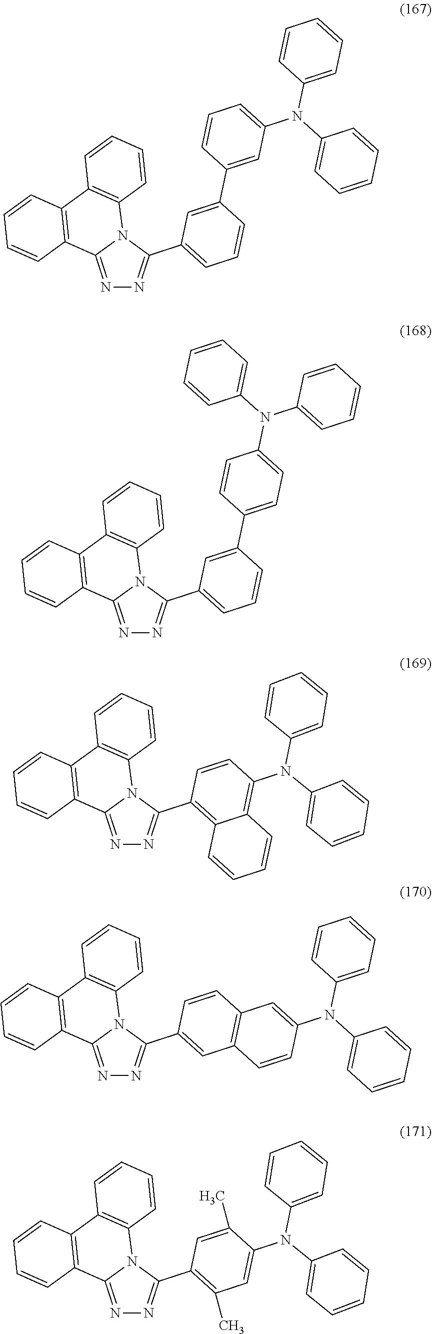 [SMILES: c1ccc(N(c2ccccc2)c2cccc(-c3cccc(-c4nnc5c6ccccc6c6ccccc6n45)c3)c2)cc1 ; c1ccc(N(c2ccccc2)c2ccc(-c3cccc(-c4nnc5c6ccccc6c6ccccc6n45)c3)cc2)cc1 ; c1ccc(N(c2ccccc2)c2ccc(-c3nnc4c5ccccc5c5ccccc5n34)c3ccccc23)cc1 ; c1ccc(N(c2ccccc2)c2ccc3cc(-c4nnc5c6ccccc6c6ccccc6n45)ccc3c2)cc1 ; Cc1cc(N(c2ccccc2)c2ccccc2)c(C)cc1-c1nnc2c3ccccc3c3ccccc3n12]

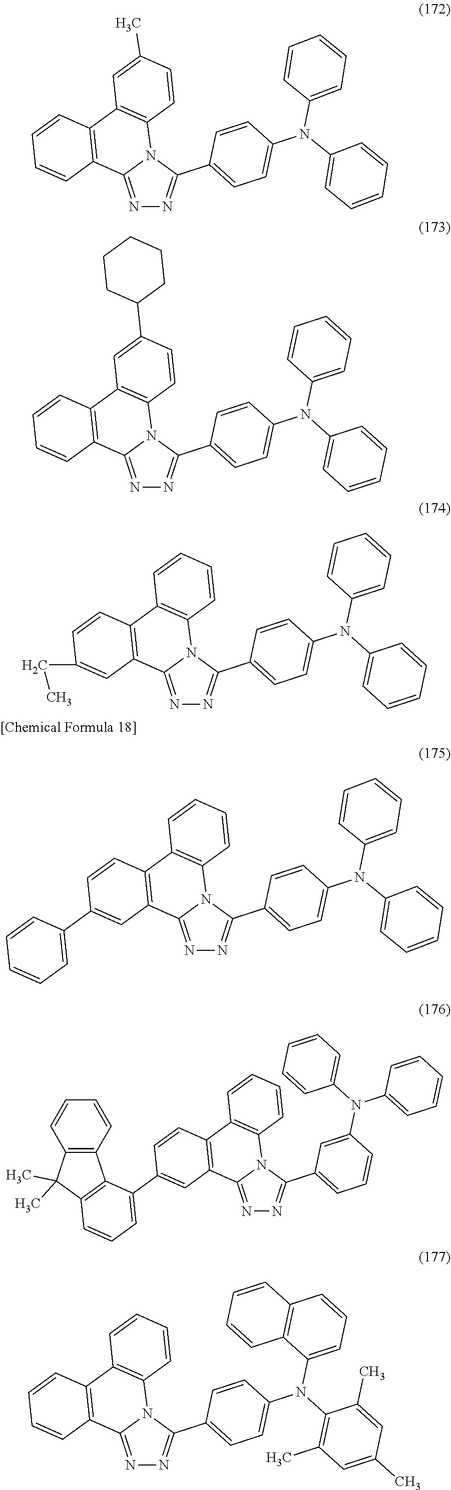

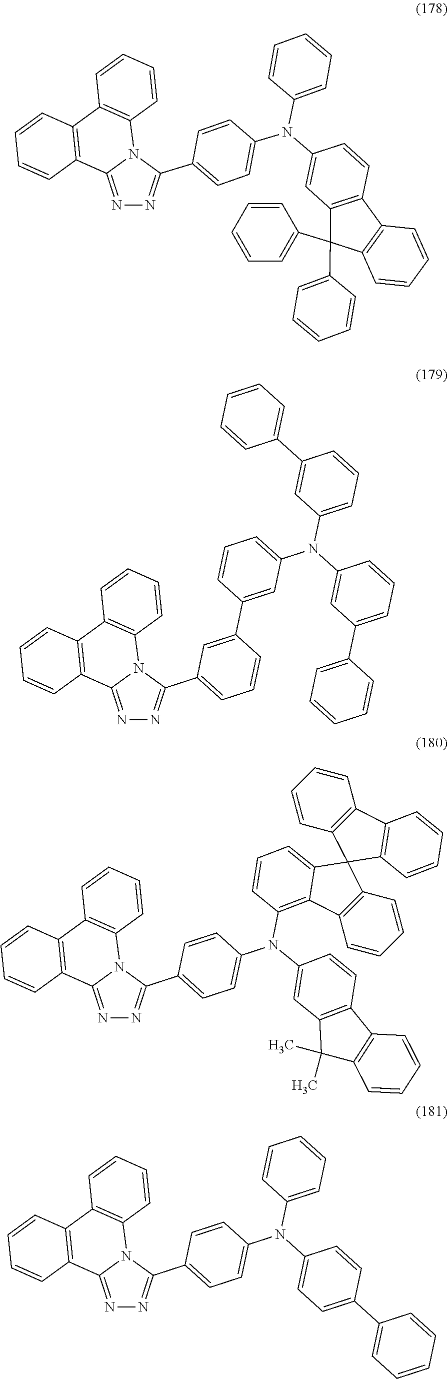 [SMILES: c1ccc(N(c2ccc(-c3nnc4c5ccccc5c5ccccc5n34)cc2)c2ccc3c(c2)C(c2ccccc2)(c2ccccc2)c2ccccc2-3)cc1 ; c1ccc(-c2cccc(N(c3cccc(-c4ccccc4)c3)c3cccc(-c4cccc(-c5nnc6c7ccccc7c7ccccc7n56)c4)c3)c2)cc1 ; CC1(C)c2ccccc2-c2ccc(N(c3ccc(-c4nnc5c6ccccc6c6ccccc6n45)cc3)c3cccc4c3-c3ccccc3C43c4ccccc4-c4ccccc43)cc21 ; c1ccc(-c2ccc(N(c3ccccc3)c3ccc(-c4nnc5c6ccccc6c6ccccc6n45)cc3)cc2)cc1]

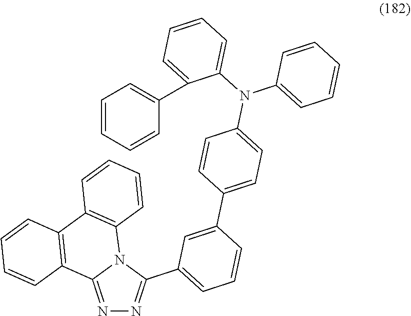 [SMILES: c1ccc(-c2ccccc2N(c2ccccc2)c2ccc(-c3cccc(-c4nnc5c6ccccc6c6ccccc6n45)c3)cc2)cc1]

Next, a method for synthesizing the organic compound of one embodiment of the present invention is exemplified.

As shown in Synthesis Scheme (A-1), the organic compound of one embodiment of the present invention represented by General Formula (G1) above can be obtained in such a manner: a halide of a 1,2,4-triazolo[4,3-f]phenanthridine derivative or imidazo[1,2-J]phenanthridine derivative, or a 1,2,4-triazolo[4,3-f]phenanthridine derivative or imidazo[1,2-J]phenanthridine derivative having triflate as a substituent (Compound 1) is coupled with an organoboron compound or a boronic acid of an aryl group having a carbazolyl group, a dibenzothiophenyl group, a dibenzofuranyl group, or a diarylamino group (Compound 2) by the Suzuki-Miyaura reaction.

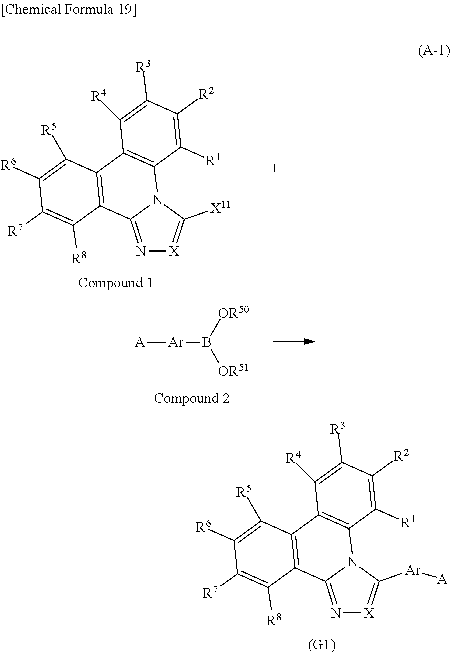

In Synthesis Scheme (A-1), X represents nitrogen or carbon. Note that when X represents carbon, the carbon may have a substituent. Ar represents a substituted or unsubstituted arylene group having 6 to 12 carbon atoms. R¹ to R⁸ each independently represent any of hydrogen, an alkyl group having 1 to 6 carbon atoms, a cyclic alkyl group having 3 to 7 carbon atoms, and a substituted or unsubstituted aryl group having 6 to 13 carbon atoms. A represents a substituted or unsubstituted diarylamino group when X represents N, and represents a substituted or unsubstituted carbazolyl group, a substituted or unsubstituted dibenzothiophenyl group, a substituted or unsubstituted dibenzofuranyl group, or a substituted or unsubstituted diarylamino group when X represents carbon. In Synthesis Scheme (A-1), R^{5′} and R⁵¹ each independently represent any of hydrogen and an alkyl group having 1 to 6 carbon atoms, and R^{5′} and R⁵¹ may be bonded to each other to form a ring. Furthermore, X¹¹ represents halogen or a triflate group.

In the case where the reaction represented by Synthesis Scheme (A-1) is performed by the Suzuki-Miyaura reaction, a palladium catalyst is preferably used; examples of the palladium catalyst include palladium(II) acetate, tetrakis(triphenylphosphine)palladium(0), and bis(triphenylphosphine)palladium(II)dichloride, and other catalysts effective for the reaction may be used. Examples of a ligand of the palladium catalyst that can be used in the above synthesis include tri(ortho-tolyl)phosphine, triphenylphosphine, and tricyclohexylphosphine. Note that other ligands of a palladium catalyst that are effective for the reaction may be used.

A base is preferably used in the reaction of Synthesis Scheme (A-1) above. Examples of a base that can be used in the reaction include an organic base typified by sodium tert-butoxide and an inorganic base typified by potassium carbonate or sodium carbonate, and other bases can also be used.

The reaction represented by Synthesis Scheme (A-1) is preferably performed using a solvent. Examples of a solvent that can be used in the reaction include a mixed solvent of toluene and water; a mixed solvent of toluene, alcohol typified by ethanol, and water; a mixed solvent of xylene and water; a mixed solvent of xylene, alcohol typified by ethanol, and water; a mixed solvent of benzene and water; a mixed solvent of benzene, alcohol typified by ethanol, and water; and a mixed solvent of water and an ether typified by ethylene glycol dimethyl ether. Note that a mixed solvent of toluene and water, a mixed solvent of toluene, ethanol, and water, or a mixed solvent of water and an ether such as ethylene glycol dimethyl ether is further preferable. In addition, other solvents effective for the reaction may be used.

In the Suzuki-Miyaura coupling reaction represented by Synthesis Scheme (A-1) above, a cross coupling reaction is performed using an organoboron compound or a boronic acid as Compound 2; and the cross coupling reaction may be performed using, other than the above, an organic aluminum, organic zirconium, organic zinc, or organic tin compound or the like.

Furthermore, in the synthesis scheme shown in Synthesis Scheme (A-1), a halide of a 1,2,4-triazolo[4,3-f]phenanthridine derivative or imidazo[1,2-f]phenanthridine derivative, or a 1,2,4-triazolo[4,3-f]phenanthridine derivative or imidazo[1,2-f]phenanthridine derivative having triflate as a substituent (Compound 1) is reacted with an organoboron compound or a boronic acid of an aryl group having a carbazolyl group, a dibenzothiophenyl group, a dibenzofuranyl group, or a diarylamino group (Compound 2). An organoboron compound or a boronic acid of a 1,2,4-triazolo[4,3-f]phenanthridine derivative or an imidazo[1,2-f]phenanthridine derivative may be reacted with a halide of an aryl group having a carbazolyl group, a dibenzothiophenyl group, a dibenzofuranyl group, or a diarylamino group, or an aryl group having a carbazolyl group, a dibenzothiophenyl group, a dibenzofuranyl group, or a diarylamino group and having triflate as a substituent.

As shown in Synthesis Scheme (A-2), the organic compound of one embodiment of the present invention represented by General Formula (G2) below can be obtained in such a manner: a halide of an imidazo[1,2-f]phenanthridine derivative or an imidazo[1,2-J]phenanthridine derivative having triflate as a substituent (Compound 3) is coupled with an organoboron compound or a boronic acid of a dibenzothiophenyl group or a dibenzofuranyl group (Compound 4) by the Suzuki-Miyaura reaction.

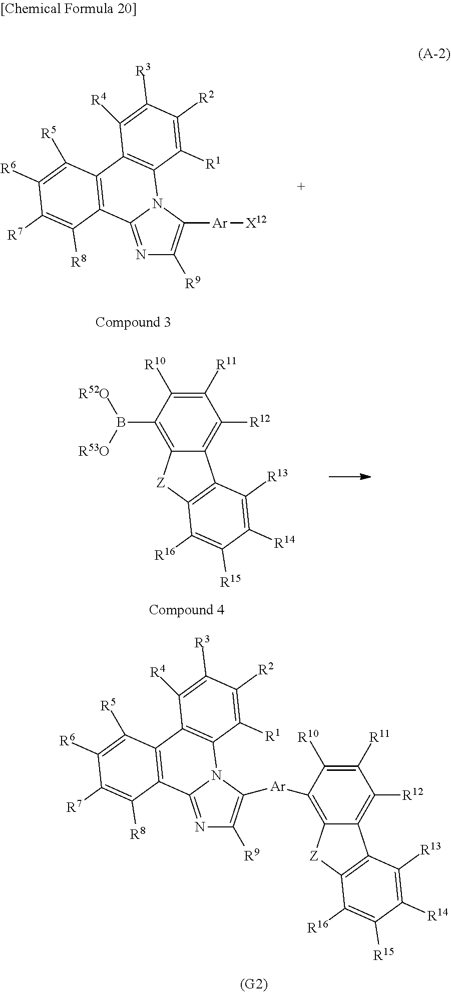

In Synthesis Scheme (A-2), Z represents oxygen or sulfur. Ar represents a substituted or unsubstituted arylene group having 6 to 12 carbon atoms. R¹ to R⁹ each independently represent any of hydrogen, an alkyl group having 1 to 6 carbon atoms, a cyclic alkyl group having 3 to 7 carbon atoms, and a substituted or unsubstituted aryl group having 6 carbon atoms to 13 carbon atoms. R¹⁰ to R¹⁶ each independently represent any of hydrogen, an alkyl group having 1 to 6 carbon atoms, a cyclic alkyl group having 3 to 7 carbon atoms, and a substituted or unsubstituted aryl group having 6 to 13 carbon atoms. In Synthesis Scheme (A-2), R⁵² and R⁵³ each independently represent any of hydrogen and an alkyl group having 1 to 6 carbon atoms, and R⁵² and R⁵³ may be bonded to each other to form a ring. Furthermore, X¹² represents halogen or a triflate group.

In the case of performing the synthesis scheme (A-2) by the Suzuki-Miyaura reaction, a palladium catalyst is preferably used; examples of the palladium catalyst include palladium(II) acetate, tetrakis(triphenylphosphine)palladium(0), and bis(triphenylphosphine)palladium(II) dichloride, and other catalysts effective for the reaction may be used. Examples of a ligand of the palladium catalyst that can be used in the above synthesis include tri(ortho-tolyl)phosphine, triphenylphosphine, and tricyclohexylphosphine. Note that other ligands of a palladium catalyst that are effective for performing the reaction may be used.

A base is preferably used in the reaction represented by Synthesis Scheme (A-2) above. Examples of a base that can be used in the reaction include an organic base typified by sodium tert-butoxide and an inorganic base typified by potassium carbonate or sodium carbonate, and other bases may be used.

The reaction represented by Synthesis Scheme (A-2) is preferably performed using a solvent. Examples of a solvent that can be used in the reaction include a mixed solvent of toluene and water; a mixed solvent of toluene, alcohol typified by ethanol, and water; a mixed solvent of xylene and water; a mixed solvent of xylene, alcohol typified by ethanol, and water; a mixed solvent of benzene and water; a mixed solvent of benzene, alcohol typified by ethanol, and water; and a mixed solvent of water and an ether typified by ethylene glycol dimethyl ether. Note that a mixed solvent of toluene and water, a mixed solvent of toluene, ethanol, and water, or a mixed solvent of water and an ether typified by ethylene glycol dimethyl ether is further preferable. In addition, other solvents effective for the reaction may be used.

In the Suzuki-Miyaura coupling reaction represented by Synthesis Scheme (A-2) above, a cross coupling reaction is performed using an organoboron compound or a boronic acid as Compound 4; and the cross coupling reaction may be performed using, other than the above, an organic aluminum, organic zirconium, organic zinc, or an organic tin compound or the like.

Furthermore, in the synthesis scheme shown in Synthesis Scheme (A-2), a halide of an imidazo[1,2-f]phenanthridine derivative or an imidazo[1,2-f]phenanthridine derivative having triflate as a substituent (Compound 3) is reacted with an organoboron compound or a boronic acid of a dibenzothiophenyl group or a dibenzofuranyl group (Compound 4). An organoboron compound or a boronic acid of an imidazo[1,2-f]phenanthridine derivative may be reacted with a halide of a dibenzothiophenyl group or dibenzofuranyl group, or a dibenzothiophenyl group or dibenzofuranyl group having triflate as a substituent.

As shown in Synthesis Scheme (A-3), the organic compound of one embodiment of the present invention represented by General Formula (G3) below can be obtained in such a manner: a halide of an imidazo[1,2-f]phenanthridine derivative or an imidazo[1,2-J]phenanthridine derivative having triflate as a substituent (Compound 5) is coupled with a carbazole derivative (Compound 6) by the Hartwig-Buchwald reaction.

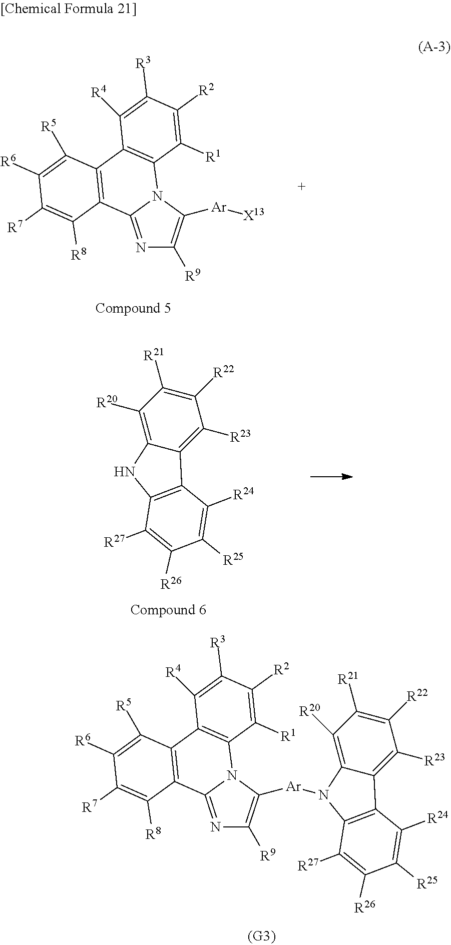

In Synthesis Scheme (A-3), Ar represents a substituted or unsubstituted arylene group having 6 to 12 carbon atoms. Furthermore, R¹ to R⁹ each independently represent any of hydrogen, an alkyl group having 1 to 6 carbon atoms, a cyclic alkyl group having 3 to 7 carbon atoms, and a substituted or unsubstituted aryl group having 6 to 13 carbon atoms. Furthermore, R²⁰ to R²⁷ each independently represent any of hydrogen, an alkyl group having 1 to 6 carbon atoms, a cyclic alkyl group having 3 to 7 carbon atoms, and a substituted or unsubstituted aryl group having 6 to 13 carbon atoms. Furthermore, X¹³ represents halogen or a triflate group.

In the case where the reaction represented by Synthesis Scheme (A-3) is performed by the Hartwig-Buchwald reaction, a palladium catalyst is preferably used; examples of the palladium catalyst include bis(dibenzylideneacetone)palladium(0) and palladium(II) acetate. Examples of a ligand of the palladium catalyst that can be used in the above synthesis include tri(tert-butyl)phosphine, tri(n-hexyl)phosphine, and tricyclohexylphosphine.

A base is preferably used in the reaction represented by Synthesis Scheme (A-3) above. Examples of a base that can be used in the reaction include an organic base typified by sodium tert-butoxide and an inorganic base typified by potassium carbonate.

The reaction represented by Synthesis Scheme (A-3) is preferably performed using a solvent. Examples of a solvent that can be used in the reaction include toluene, xylene, benzene, and tetrahydrofuran.

Note that for the reaction represented by Synthesis Scheme (A-3), reaction mechanisms typified by the Ullmann reaction may be used other than the Hartwig-Buchwald reaction.

As shown in Synthesis Scheme (A-4), the organic compound of one embodiment of the present invention represented by General Formula (G4) below can be obtained in such a manner: a halide of a 1,2,4-triazolo[4,3-f]phenanthridine derivative or imidazo[1,2-J]phenanthridine derivative, or a 1,2,4-triazolo[4,3-f]phenanthridine derivative or imidazo[1,2-J]phenanthridine derivative having triflate as a substituent (Compound 7) is coupled with a diarylamine derivative (Compound 8) by the Hartwig-Buchwald reaction.

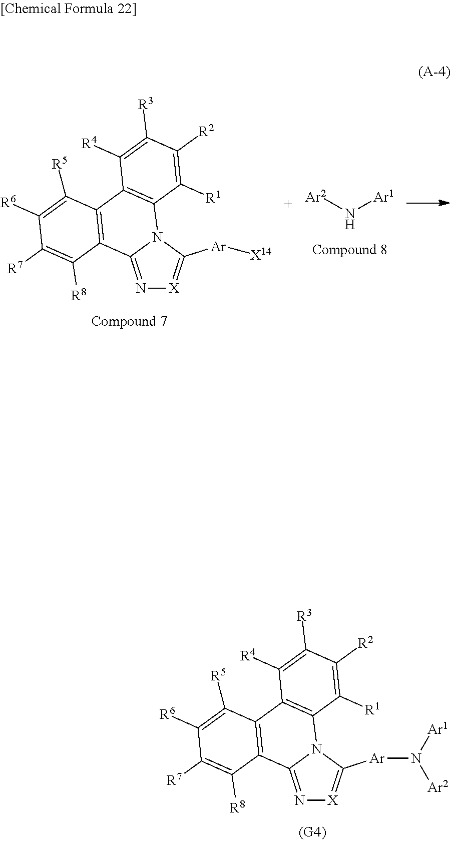

In Synthesis Scheme (A-4), X represents nitrogen or carbon. Note that when X is carbon, X may have a substituent. Ar represents a substituted or unsubstituted arylene group having 6 to 12 carbon atoms. Ar¹ and Ar² each independently represent any substituted or unsubstituted aryl group having 6 to 13 carbon atoms. R¹ to R⁸ each independently represent any of hydrogen, an alkyl group having 1 to 6 carbon atoms, a cyclic alkyl group having 3 to 7 carbon atoms, and a substituted or unsubstituted aryl group having 6 to 13 carbon atoms. Furthermore, X¹⁴ represents halogen or a triflate group.

In the case where the reaction represented by Synthesis Scheme (A-4) is performed by the Hartwig-Buchwald reaction, a palladium catalyst is preferably used; examples of the palladium catalyst include bis(dibenzylideneacetone)palladium(0) and palladium(II) acetate. Examples of a ligand of the palladium catalyst that can be used in the above synthesis include tri(tert-butyl)phosphine, tri(n-hexyl)phosphine, and tricyclohexylphosphine.

A base is preferably used in the reaction represented by Synthesis Scheme (A-4) above. Examples of a base that can be used in the reaction include an organic base typified by sodium tert-butoxide and an inorganic base typified by potassium carbonate.

The reaction represented by Synthesis Scheme (A-4) is preferably performed using a solvent. Examples of a solvent that can be used in the reaction include toluene, xylene, benzene, and tetrahydrofuran.

Note that for the reaction represented by Synthesis Scheme (A-4), reaction mechanisms typified by the Ullmann reaction may be used other than the Hartwig-Buchwald reaction.

Note that the compound described in this embodiment can be used in an appropriate combination with any of the structures described in the other embodiments.

### Embodiment 2

In this embodiment, a light-emitting device of one embodiment of the present invention is described.

FIG. TA is a diagram illustrating a light-emitting device of one embodiment of the present invention. The light-emitting device of one embodiment of the present invention includes a first electrode 101, a second electrode 102, and an EL layer 103. The EL layer 103 includes the organic compound described in Embodiment 1.

The EL layer 103 includes a light-emitting layer 113, and the light-emitting layer 113 contains a light-emitting material. The organic compound described in Embodiment 1 is preferably used as a material that disperses the light-emitting material into the light-emitting layer 113. Note that the light-emitting layer 113 may contain another material.

The light-emitting layer 113 may be formed by co-evaporating the organic compound described in Embodiment 1 and a hole-transport material. In that case, the organic compound described in Embodiment 1 and the hole-transport material may be configured to form an exciplex. The exciplex having an appropriate emission wavelength allows efficient energy transfer to the light-emitting material, achieving a light-emitting device with a high efficiency and along lifetime.

Note that although FIG. TA illustrates a hole-injection layer 111, a hole-transport layer 112, an electron-transport layer 114, and an electron-injection layer 115 in the EL layer 103 in addition to the light-emitting layer 113, the structure of the light-emitting device is not limited thereto. Any of these layers may be omitted or a layer having another function may be included.

The organic compound described in Embodiment 1 exhibits a high electron-transport property and thus is effectively used for the electron-transport layer 114.

Next, examples of specific structures and materials of the aforementioned light-emitting device will be described. As described above, the light-emitting device of one embodiment of the present invention includes, between the pair of electrodes of the first electrode 101 and the second electrode 102, the EL layer 103 including a plurality of layers; the EL layer 103 includes the organic compound disclosed in Embodiment 1 in any of the layers.

The first electrode 101 is preferably formed using a metal, an alloy, or a conductive compound having a high work function (specifically, 4.0 eV or more), a mixture thereof, or the like. Specifically, for example, indium oxide-tin oxide (ITO: Indium Tin Oxide), indium oxide-tin oxide containing silicon or silicon oxide, indium oxide-zinc oxide, indium oxide containing tungsten oxide and zinc oxide (IWZO), and the like can be given. These conductive metal oxide films are usually formed by a sputtering method but may also be formed by application of a sol-gel method or the like. An example of the formation method is a method in which indium oxide-zinc oxide is formed by a sputtering method using a target in which 1 to 20 wt % zinc oxide is added to indium oxide. Indium oxide containing tungsten oxide and zinc oxide (IWZO) can also be formed by a sputtering method using a target containing 0.5 to 5 wt % tungsten oxide and 0.1 to 1 wt % zinc oxide with respect to indium oxide. Alternatively, gold (Au), platinum (Pt), nickel (Ni), tungsten (W), chromium (Cr), molybdenum (Mo), iron (Fe), cobalt (Co), copper (Cu), palladium (Pd), a nitride of a metal material (such as titanium nitride), and the like can be given. Graphene can also be used. Note that when a composite material described later is used for a layer that is in contact with the first electrode 101 in the EL layer 103, an electrode material can be selected regardless of its work function.

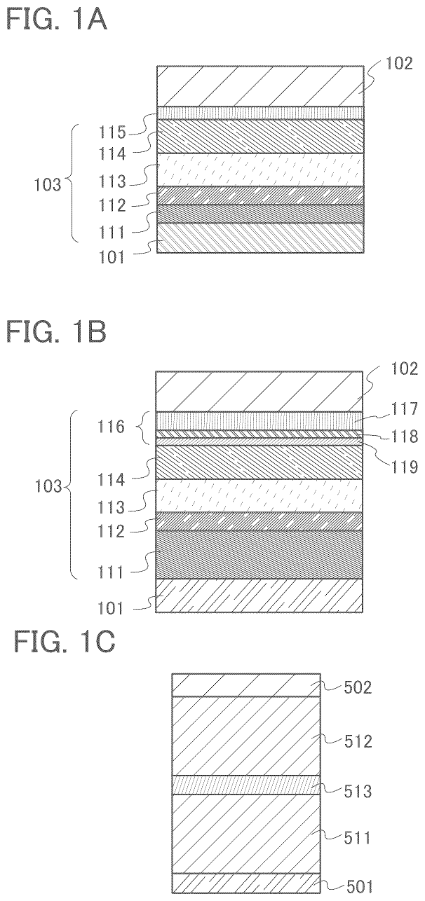

The EL layer 103 preferably has a stacked-layer structure. There is no particular limitation on the stacked-layer structure, and various layer structures such as a hole-injection layer, a hole-transport layer, alight-emitting layer, an electron-transport layer, an electron-injection layer, a carrier-blocking layer, an exciton-blocking layer, and a charge-generation layer can be employed. In this embodiment, two kinds of structures are described: the structure including the electron-transport layer 114 and the electron-injection layer 115 in addition to the hole-injection layer 111, the hole-transport layer 112, and the light-emitting layer 113 as illustrated in FIG. 1A; and the structure including the electron-transport layer 114 and a charge-generation layer 116 in addition to the hole-injection layer 111, the hole-transport layer 112, and the light-emitting layer 113 as illustrated in FIG. 1B. Materials forming the layers are specifically described below.

The hole-injection layer 111 contains a substance having an acceptor property. Either an organic compound or an inorganic compound can be used as the substance having an acceptor property.

As the substance having an acceptor property, a compound having an electron-withdrawing group (a halogen group, a cyano group, and the like) can be used; 7,7,8,8-tetracyano-2,3,5,6-tetrafluoroquinodimethane (abbreviation: F₄-TCNQ), chloranil, 2,3,6,7,10,11-hexacyano-1,4,5,8,9,12-hexaazatriphenylene (abbreviation: HAT-CN), 1,3,4,5,7,8-hexafluorotetracyano-naphthoquinodimethane (abbreviation: F6-TCNNQ), 2-(7-dicyanomethylene-1,3,4,5,6,8,9,10-octafluoro-7H-pyren-2-ylidene)malononitrile, and the like can be given. A compound in which electron-withdrawing groups are bonded to a condensed aromatic ring having a plurality of heteroatoms, such as HAT-CN, is particularly preferable because it is thermally stable. A [3]radialene derivative having an electron-withdrawing group (in particular, a cyano group, a halogen group such as a fluoro group, and the like) has a very high electron-accepting property and thus is preferable. Specific examples include α,α′,α″-1,2,3-cyclopropanetriylidenetris[4-cyano-2,3,5,6-tetrafluorobenzeneacetonitrile], α,α′,α″-1,2,3-cyclopropanetriylidenetris[2,6-dichloro-3,5-difluoro-4-(trifluoromethyl)benzeneacetonitrile], and α,α′,α″-1,2,3-cyclopropanetriylidenetris[2,3,4,5,6-pentafluorobenzeneacetonitrile]. As the substance having an acceptor property, a transition metal oxide typified by molybdenum oxide, vanadium oxide, ruthenium oxide, tungsten oxide, manganese oxide, or the like can be used, other than the above-described organic compounds. Alternatively, the hole-injection layer 111 can be formed using phthalocyanine (abbreviation: H₂Pc), a phthalocyanine-based complex compound typified by copper phthalocyanine (CuPc), an aromatic amine compound typified by 4,4′-bis[N-(4-diphenylaminophenyl)-N-phenylamino]biphenyl (abbreviation: DPAB) or N,N-bis{4-[bis(3-methylphenyl)amino]phenyl}-N,N-diphenyl-(1,1′-biphenyl)-4,4′-diamine (abbreviation: DNTPD), or a high molecular compound typified by poly(3,4-ethylenedioxythiophene)/poly(styrenesulfonic acid) (PEDOT/PSS). The substance having an acceptor property can extract electrons from an adjacent hole-transport layer (or hole-transport material) by the application of an electric field.

Alternatively, a composite material in which a material having a hole-transport property contains the above-described acceptor substance can be used for the hole-injection layer 111. By using a composite material in which a material having a hole-transport property contains an acceptor substance, a material used to form an electrode can be selected regardless of its work function. In other words, besides a material having a high work function, a material having a low work function can also be used for the first electrode 101.

As the material having a hole-transport property used for the composite material, any of a variety of organic compounds such as aromatic amine compounds, carbazole derivatives, aromatic hydrocarbons, and high molecular compounds (oligomers, dendrimers, or polymers) can be used. Note that the material having a hole-transport property used for the composite material preferably has a hole mobility of 1×10⁻⁶ cm²/Vs or higher. Organic compounds which can be used as the material having a hole-transport property in the composite material are specifically given below.

Examples of the aromatic amine compounds that can be used for the composite material include N,N′-di(p-tolyl)-N,N′-diphenyl-p-phenylenediamine (abbreviation: DTDPPA), 4,4′-bis[N-(4-diphenylaminophenyl)-N-phenylamino]biphenyl (abbreviation: DPAB), N,N′-bis{4-[bis(3-methylphenyl)amino]phenyl}-N,N-diphenyl-(1,1′-biphenyl)-4,4′-diamine (abbreviation: DNTPD), and 1,3,5-tris[N-(4-diphenylaminophenyl)-N-phenylamino]benzene (abbreviation: DPA3B). Specific examples of the carbazole derivative include 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), 3-[N-(1-naphthyl)-N-(9-phenylcarbazol-3-yl)amino]-9-phenylcarbazole (abbreviation: PCzPCN1), 4,4′-di(N-carbazolyl)biphenyl (abbreviation: CBP), 1,3,5-tris[4-(N-carbazolyl)phenyl]benzene (abbreviation: TCPB), 9-[4-(10-phenyl-9-anthracenyl)phenyl]-9H-carbazole (abbreviation: CzPA), and 1,4-bis[4-(N-carbazolyl)phenyl]-2,3,5,6-tetraphenylbenzene. Examples of the aromatic hydrocarbon include 2-tert-butyl-9,10-di(2-naphthyl)anthracene (abbreviation: t-BuDNA), 2-tert-butyl-9,10-di(1-naphthyl)anthracene, 9,10-bis(3,5-diphenylphenyl)anthracene (abbreviation: DPPA), 2-tert-butyl-9,10-bis(4-phenylphenyl)anthracene (abbreviation: t-BuDBA), 9,10-di(2-naphthyl)anthracene (abbreviation: DNA), 9,10-diphenylanthracene (abbreviation: DPAnth), 2-tert-butylanthracene (abbreviation: t-BuAnth), 9,10-bis(4-methyl-1-naphthyl)anthracene (abbreviation: DMNA), 2-tert-butyl-9,10-bis[2-(1-naphthyl)phenyl]anthracene, 9,10-bis[2-(1-naphthyl)phenyl]anthracene, 2,3,6,7-tetramethyl-9,10-di(1-naphthyl)anthracene, 2,3,6,7-tetramethyl-9,10-di(2-naphthyl)anthracene, 9,9′-bianthryl, 10,10′-diphenyl-9,9′-bianthryl, 10,10′-bis(2-phenylphenyl)-9,9′-bianthryl, 10,10′-bis[(2,3,4,5,6-pentaphenyl)phenyl]-9,9′-bianthryl, anthracene, tetracene, rubrene, perylene, and 2,5,8,11-tetra(tert-butyl)perylene. Other examples include pentacene and coronene. The aromatic hydrocarbon may have a vinyl skeleton. Examples of the aromatic hydrocarbon having a vinyl group include 4,4′-bis(2,2-diphenylvinyl)biphenyl (abbreviation: DPVBi) and 9,10-bis[4-(2,2-diphenylvinyl)phenyl]anthracene (abbreviation: DPVPA).

Other examples include high molecular compounds typified by poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide] (abbreviation: PTPDMA), and poly[N,N′-bis(4-butylphenyl)-N,N′-bis(phenyl)benzidine] (abbreviation: Poly-TPD).

The material having a hole-transport property used for the composite material further preferably has any of a carbazole skeleton, a dibenzofuran skeleton, a dibenzothiophene skeleton, and an anthracene skeleton. In particular, an aromatic amine having a substituent that includes a dibenzofuran ring or a dibenzothiophene ring, an aromatic monoamine that has a naphthalene ring, or an aromatic monoamine in which a 9-fluorenyl group is bonded to nitrogen of the amine through an arylene group may be used. Note that these second organic compounds are preferably substances having an N,N-bis(4-biphenyl)amino group because a light-emitting device with a long lifetime can be manufactured. Specific examples of the above second organic compound include N-(4-biphenyl)-6,N-diphenylbenzo[b]naphtho[1,2-d]furan-8-amine (abbreviation: BnfABP), N,N-bis(4-biphenyl)-6-phenylbenzo[b]naphtho[1,2-d]furan-8-amine (abbreviation: BBABnf), 4,4′-bis(6-phenylbenzo[b]naphtho[1,2-d]furan-8-yl)-4″-phenyltriphenylamine (abbreviation: BnfBB1BP), N,N-bis(4-biphenyl)benzo[b]naphtho[1,2-d]furan-6-amine (abbreviation: BBABnf(6)), N,N-bis(4-biphenyl)benzo[b]naphtho[1,2-d]furan-8-amine (abbreviation: BBABnf(8)), N,N-bis(4-biphenyl)benzo[b]naphtho[2,3-d]furan-4-amine (abbreviation: BBABnf(II)(4)), N,N-bis[4-(dibenzofuran-4-yl)phenyl]-4-amino-p-terphenyl (abbreviation: DBfBB1TP), N-[4-(dibenzothiophen-4-yl)phenyl]-N-phenyl-4-biphenylamine (abbreviation: ThBA1BP), 4-(2-naphthyl)-4′,4″-diphenyltriphenylamine (abbreviation: BBAβNB), 4-[4-(2-naphthyl)phenyl]-4′,4″-diphenyltriphenylamine (abbreviation: BBAβNBi), 4,4′-diphenyl-4″-(6;1′-binaphthyl-2-yl)triphenylamine (abbreviation: BBAαNβNB), 4,4′-diphenyl-4″-(7;1′-binaphthyl-2-yl)triphenylamine (abbreviation: BBAαNβNB-03), 4,4′-diphenyl-4″-(7-phenyl)naphthyl-2-yltriphenylamine (abbreviation: BBAPβNB-03), 4,4′-diphenyl-4″-(6;2′-binaphthyl-2-yl)triphenylamine (abbreviation: BBA(βN2)B), 4,4′-diphenyl-4″-(7;2′-binaphthyl-2-yl)triphenylamine (abbreviation: BBA(βN2)B-03), 4,4′-diphenyl-4″-(4;2′-binaphthyl-1-yl)triphenylamine (abbreviation: BBAβNαNB), 4,4′-diphenyl-4″-(5;2′-binaphthyl-1-yl)triphenylamine (abbreviation: BBAβNαNB-02), 4-(4-biphenylyl)-4′-(2-naphthyl)-4″-phenyltriphenylamine (abbreviation: TPBiAβNB), 4-(3-biphenylyl)-4′-[4-(2-naphthyl)phenyl]-4″-phenyltriphenylamine (abbreviation: mTPBiAβNBi), 4-(4-biphenylyl)-4′-[4-(2-naphthyl)phenyl]-4″-phenyltriphenylamine (abbreviation: TPBiAβNBi), 4-phenyl-4′-(1-naphthyl)triphenylamine (abbreviation: αNBA1BP), 4,4′-bis(1-naphthyl)triphenylamine (abbreviation: αNBB1BP), 4,4′-diphenyl-4″-[4′-(carbazol-9-yl)biphenyl-4-yl]triphenylamine (abbreviation: YGTBi1BP), 4′-[4-(3-phenyl-9H-carbazol-9-yl)phenyl]tris(1,1′-biphenyl-4-yl)amine (abbreviation: YGTBi1BP-02), 4-[4′-(carbazol-9-yl)biphenyl-4-yl]-4′-(2-naphthyl)-4″-phenyltriphenylamine (abbreviation: YGTBiβNB), N-[4-(9-phenyl-9H-carbazol-3-yl)phenyl]-N-[4-(1-naphthyl)phenyl]-9,9′-spirobi(9H-fluoren)-2-amine (abbreviation: PCBNBSF), N,N-bis([1,1′-biphenyl)-4-yl)-9,9′-spirobi[9H-fluoren]-2-amine (abbreviation: BBASF), N,N-bis([1,1′-biphenyl]-4-yl)-9,9′-spirobi[9H-fluoren]-4-amine (abbreviation: BBASF(4)), N-(1,1′-biphenyl-2-yl)-N-(9,9-dimethyl-9H-fluoren-2-yl)-9,9′-spirobi(9H-fluoren)-4-amine (abbreviation: oFBiSF), N-(4-biphenyl)-N-(9,9-dimethyl-9H-fluoren-2-yl)dibenzofuran-4-amine (abbreviation: FrBiF), N-[4-(1-naphthyl)phenyl]-N-[3-(6-phenyldibenzofuran-4-yl)phenyl]-1-naphthylamine (abbreviation: mPDBfBNBN), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-[4-(9-phenylfluoren-9-yl)phenyl]triphenylamine (abbreviation: BPAFLBi), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), N-phenyl-N-[4-(9-phenyl-9H-carbazol-3-yl)phenyl]-9,9′-spirobi[9H-fluoren]-2-amine (abbreviation: PCBASF), N-(1,1′-biphenyl-4-yl)-N-[4-(9-phenyl-9H-carbazol-3-yl)phenyl]-9,9-dimethyl-9H-fluoren-2-amine (abbreviation: PCBBiF), N,N-bis(9,9-dimethyl-9H-fluoren-2-yl)-9,9′-spirobi-9H-fluoren-4-amine, N,N-bis(9,9-dimethyl-9H-fluoren-2-yl)-9,9′-spirobi-9H-fluoren-3-amine, N,N-bis(9,9-dimethyl-9H-fluoren-2-yl)-9,9′-spirobi-9H-fluoren-2-amine, and N,N-bis(9,9-dimethyl-9H-fluoren-2-yl)-9,9′-spirobi-9H-fluoren-1-amine.

Note that it is further preferable that the material having a hole-transport property used for the composite material have a relatively deep HOMO level greater than or equal to −5.7 eV and less than or equal to −5.4 eV. The relatively deep HOMO level of the material having a hole-transport property used for the composite material makes it easy to inject holes into the hole-transport layer 112 and to obtain a light-emitting device with a long lifetime.

Note that mixing the above composite material with a fluoride of an alkali metal or an alkaline earth metal (the proportion of fluorine atoms in the layer is preferably greater than or equal to 20%) can lower the refractive index of the layer. This also enables a layer with a low refractive index to be formed in the EL layer 103, leading to higher external quantum efficiency of the light-emitting device.

The formation of the hole-injection layer 111 can improve the hole-injection property, whereby a light-emitting device having a low driving voltage can be obtained. The organic compound having an acceptor property is an easy-to-use material because evaporation is easy and its film can be easily formed.

The hole-transport layer 112 is formed containing a material having a hole-transport property. The material having a hole-transport property preferably has a hole mobility higher than or equal to 1×10⁻⁶ cm²/Vs. Examples of the material having a hole-transport property include a compound having a heteroaromatic skeleton, such as 4,4′-bis[N-(1-naphthyl)-N-phenylamino]biphenyl (abbreviation: NPB), N,N-bis(3-methylphenyl)-N,N-diphenyl-[1,1′-biphenyl]-4,4′-diamine (abbreviation: TPD), 4,4′-bis[N-(spiro-9,9′-bifluoren-2-yl)-N-phenylamino]biphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)-triphenylamine (abbreviation: PCBANB), 4,4′-di(1-naphthyl)-4″-(9-phenyl-9H-carbazol-3-yl)triphenylamine (abbreviation: PCBNBB), 9,9-dimethyl-N-phenyl-N-[4-(9-phenyl-9H-carbazol-3-yl)phenyl]fluoren-2-amine (abbreviation: PCBAF), or N-phenyl-N-[4-(9-phenyl-9H-carbazol-3-yl)phenyl]-9,9′-spirobi[9H-fluoren]-2-amine (abbreviation: PCBASF), a compound having a carbazole skeleton, such as 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), or 3,3′-bis(9-phenyl-9H-carbazole) (abbreviation: PCCP), a compound having a thiophene skeleton, such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), or 4-[4-(9-phenyl-9H-fluoren-9-yl)phenyl]-6-phenyldibenzothiophene (abbreviation: DBTFLP-IV), and a compound having a furan skeleton, such as 4,4′,4″-(benzene-1,3,5-triyl)tri(dibenzofuran) (abbreviation: DBF3P-II) or 4-{3-[3-(9-phenyl-9H-fluoren-9-yl)phenyl]phenyl}dibenzofuran (abbreviation: mmDBFFLBi-II). Among the above, the compound having an aromatic amine skeleton and the compound having a carbazole skeleton are preferable because these have favorable reliability, have high hole-transport properties, and contribute to a reduction in driving voltage. Note that any of the substances given as examples of the material having a hole-transport property that is used for the composite material for the hole-injection layer 111 can also be suitably used as the material included in the hole-transport layer 112.

The light-emitting layer 113 contains a light-emitting substance and a host material. The light-emitting layer 113 may additionally contain another material. Furthermore, the light-emitting layer 113 may be a stack of two layers with different compositions.

The light-emitting substance may be fluorescent substances, phosphorescent substances, substances exhibiting thermally activated delayed fluorescence (TADF), or other light-emitting substances.

Examples of a material that can be used as a fluorescent substance in the light-emitting layer 113 include 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N-diphenyl-N,N-bis[4-(9-phenyl-9H-fluoren-9-yl)phenyl]pyrene-1,6-diamine (abbreviation: 1,6FLPAPm), N,N-bis(3-methylphenyl)-N,N′-bis[3-(9-phenyl-9H-fluoren-9-yl)phenyl]pyrene-1,6-diamine (abbreviation: 1,6mMemFLPAPrn), N,N-bis[4-(9H-carbazol-9-yl)phenyl]-N,N-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), perylene, 2,5,8,11-tetra-(tert-butyl)perylene (abbreviation: TBP), 4-(10-phenyl-9-anthryl)-4′-(9-phenyl-9H-carbazol-3-yl)triphenylamine (abbreviation: PCBAPA), N,N″-(2-tert-butylanthracene-9,10-diyldi-4,1-phenylene)bis[N,N′,N′-triphenyl-1,4-phenylenediamine] (abbreviation: DPABPA), N,9-diphenyl-N-[4-(9,10-diphenyl-2-anthryl)phenyl]-9H-carbazol-3-amine (abbreviation: 2PCAPPA), N-[4-(9,10-diphenyl-2-anthryl)phenyl]-N,N,N-triphenyl-1,4-phenylenediamine (abbreviation: 2DPAPPA), N,N,N,N,N′,N′,N″,N″-octaphenyldibenzo[g,p]chrysene-2,7,10,15-tetraamine (abbreviation: DBC1), coumarin 30, N-(9,10-diphenyl-2-anthryl)-N,9-diphenyl-9H-carbazol-3-amine (abbreviation: 2PCAPA), N-[9,10-bis(1,1′-biphenyl-2-yl)-2-anthryl]-N,9-diphenyl-9H-carbazol-3-amine (abbreviation: 2PCABPhA), N-(9,10-diphenyl-2-anthryl)-N,N′,N″-triphenyl-1,4-phenylenediamine (abbreviation: 2DPAPA), N-[9,10-bis(1,1′-biphenyl-2-yl)-2-anthryl]-N,N′,N′-triphenyl-1,4-phenylenediamine (abbreviation: 2DPABPhA), 9,10-bis(1,1′-biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N-diphenylquinacridone (abbreviation: DPQd), rubrene, 5,12-bis(1,1′-biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2-(2,3,6,7-tetrahydro-1H,5H-benzo[ij]quinolizin-9-yl)ethenyl]-4H-pyran-4-ylidene}propanedinitrile (abbreviation: DCM2), N,N,N,N-tetrakis(4-methylphenyl)tetracene-5,11-diamine (abbreviation: p-mPhTD), 7,14-diphenyl-N,N,N,N-tetrakis(4-methylphenyl)acenaphtho[1,2-a]fluoranthene-3,10-diamine (abbreviation: p-mPhAFD), 2-{2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis{2-[4-(dimethylamino)phenyl]ethenyl}-4H-pyran-4-ylidene)propanedinitrile (abbreviation: BisDCM), 2-{2,6-bis[2-(8-methoxy-1,1,7,7-tetramethyl-2,3,6,7-tetrahydro-1H,5H-benzo[ij]quinolizin-9-yl)ethenyl]-4H-pyran-4-ylidene}propanedinitrile (abbreviation: BisDCJTM), N,N-(pyrene-1,6-diyl)bis[(6,N-diphenylbenzo[b]naphtho[1,2-d]furan)-8-amine](abbreviation: 1,6BnfAPrn-03), 3,10-bis[N-(9-phenyl-9H-carbazol-2-yl)-N-phenylamino]naphtho[2,3-b;6,7-b′]bisbenzofuran (abbreviation: 3,10PCA2Nbf(IV)-02), and 3,10-bis[N-(dibenzofuran-3-yl)-N-phenylamino]naphtho[2,3-b;6,7-b′]bisbenzofuran (abbreviation: 3,10FrA2Nbf(IV)-02). In particular, a condensed aromatic diamine compound typified by a pyrenediamine compound such as 1,6FLPAPrn, 1,6mMemFLPAPm, and 1,6BnfAPm-03 is preferable because of its high hole-trapping property, high emission efficiency, and high reliability. Fluorescent substances other than those can also be used.

In the case where a phosphorescent substance is used as a light-emitting substance in the light-emitting layer 113, examples of a material that can be used include an organometallic iridium complex having a 4H-triazole skeleton, such as tris{2-[5-(2-methylphenyl)-4-(2,6-dimethylphenyl)-4H-1,2,4-triazol-3-yl-κN²]phenyl-κC}iridium(III) (abbreviation: [Ir(mpptz-dmp)₃]), tris(5-methyl-3,4-diphenyl-4H-1,2,4-triazolato)iridium(III) (abbreviation: [Ir(Mptz)₃]), and tris[4-(3-biphenyl)-5-isopropyl-3-phenyl-4H-1,2,4-triazolato]iridium(III) (abbreviation: [Ir(iPrptz-3b)₃]); an organometallic iridium complex having a 1H-triazole skeleton, such as tris[3-methyl-1-(2-methylphenyl)-5-phenyl-1H-1,2,4-triazolato]iridium(III) (abbreviation: [Ir(Mptzl-mp)₃]) and tris(1-methyl-5-phenyl-3-propyl-TH-1,2,4-triazolato)iridium(III) (abbreviation: [Ir(Prptz1-Me)₃]); an organometallic iridium complex having an imidazole skeleton, such as fac-tris[1-(2,6-diisopropylphenyl)-2-phenyl-1H-imidazole]iridium(III) (abbreviation: [Ir(iPrpmi)₃]) and tris[3-(2,6-dimethylphenyl)-7-methylimidazo[1,2-f]phenanthridinato]iridium(III) (abbreviation: [Ir(dmpimpt-Me)₃]); and an organometallic iridium complex in which a phenylpyridine derivative having an electron-withdrawing group is a ligand, such as bis[2-(4′,6′-difluorophenyl)pyridinato-N,C²]iridium(III) tetrakis(1-pyrazolyl)borate (abbreviation: FIr6), bis[2-(4′,6′-difluorophenyl)pyridinato-N,C^{2′}]iridium(III) picolinate (abbreviation: FIrpic), bis{2-[3′,5′-bis(trifluoromethyl)phenyl]pyridinato-N,C^{2′}}iridium(III) picolinate (abbreviation: [Ir(CF₃ppy)₂(pic)]), and bis[2-(4′,6′-difluorophenyl)pyridinato-N,C²]iridium(III) acetylacetonate (abbreviation: FIr(acac)). These are compounds exhibiting blue phosphorescent light, and are compounds having an emission spectrum peak at 440 nm to 520 nm.

Examples also include an organometallic iridium complex having a pyrimidine skeleton, such as tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbomyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), or (acetylacetonato)bis(4,6-diphenylpyrimidinato)iridium(III) (abbreviation: [Ir(dppm)₂(acac)]), an organometallic iridium complex having a pyrazine skeleton, such as (acetylacetonato)bis(3,5-dimethyl-2-phenylpyrazinato)iridium(III) (abbreviation: [Ir(mppr-Me)₂(acac)]) or (acetylacetonato)bis(5-isopropyl-3-methyl-2-phenylpyrazinato)iridium(III) (abbreviation: [Ir(mppr-iPr)₂(acac)]), an organometallic iridium complex having a pyridine skeleton, such as tris(2-phenylpyridinato-N,C²′)iridium(III) (abbreviation: [Ir(ppy)₃]), bis(2-phenylpyridinato-N,C²′)iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C²′)iridium(III) (abbreviation: [Ir(pq)₃]), bis(2-phenylquinolinato-N,C²′)iridium(III) acetylacetonate (abbreviation: [Ir(pq)₂(acac)]), and a rare earth metal complex such as tris(acetylacetonato)(monophenanthroline)terbium(III) (abbreviation: [Tb(acac)₃(Phen)]). These are compounds that mainly exhibit green phosphorescent light, and have an emission spectrum peak at 500 nm to 600 nm. Note that an organometallic iridium complex having a pyrimidine skeleton is particularly preferable because of its distinctively high reliability and emission efficiency.

Examples also include an organometallic iridium complex having a pyrimidine skeleton, such as (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), or bis[4,6-di(naphthalen-1-yl)pyrimidinato](dipivaloylmethanato)iridium(III) (abbreviation: [Ir(d1npm)₂(dpm)]), an organometallic iridium complex having a pyrazine skeleton, such as (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), or (acetylacetonato)bis[2,3-bis(4-fluorophenyl)quinoxalinato]iridium(III) (abbreviation: [Ir(Fdpq)₂(acac)]), an organometallic iridium complex having a pyridine skeleton, such as tris(1-phenylisoquinolinato-N,C²′)iridium(III) (abbreviation: [Ir(piq)₃]) or bis(1-phenylisoquinolinato-N,C²′)iridium(III) acetylacetonate (abbreviation: [Ir(piq)₂(acac)]), a platinum complex such as 2,3,7,8,12,13,17,18-octaethyl-21H,23H-porphyrin platinum(II) (abbreviation: PtOEP), and a rare earth metal complex such as tris(1,3-diphenyl-1,3-propanedionato)(monophenanthroline)europium(III) (abbreviation: [Eu(DBM)₃(Phen)]) or tris[1-(2-thenoyl)-3,3,3-trifluoroacetonato](monophenanthroline)europium(III) (abbreviation: [Eu(TTA)₃(Phen)]). These are compounds exhibiting red phosphorescent light, and have an emission spectrum peak at 600 nm to 700 nm. Furthermore, from the organometallic iridium complex having a pyrazine skeleton, red light emission with favorable chromaticity can be obtained.

Besides the above-described phosphorescent compounds, other known phosphorescent substances may be selected and used.

As the TADF material, a fullerene, a derivative thereof, an acridine, a derivative thereof, an eosin derivative, or the like can be used. Other examples include a metal-containing porphyrin containing magnesium (Mg), zinc (Zn), cadmium (Cd), tin (Sn), platinum (Pt), indium (In), palladium (Pd), or the like. Examples of the metal-containing porphyrin include a protoporphyrin-tin fluoride complex (SnF₂(Proto IX)), a mesoporphyrin-tin fluoride complex (SnF₂(Meso IX)), a hematoporphyrin-tin fluoride complex (SnF₂(Hemato IX)), a coproporphyrin tetramethyl ester-tin fluoride complex (SnF₂(Copro III-4Me)), an octaethylporphyrin-tin fluoride complex (SnF₂(OEP)), an etioporphyrin-tin fluoride complex (SnF₂(Etio I)), and an octaethylporphyrin-platinum chloride complex (PtCl₂(OEP)), which are represented by the following structural formulae.

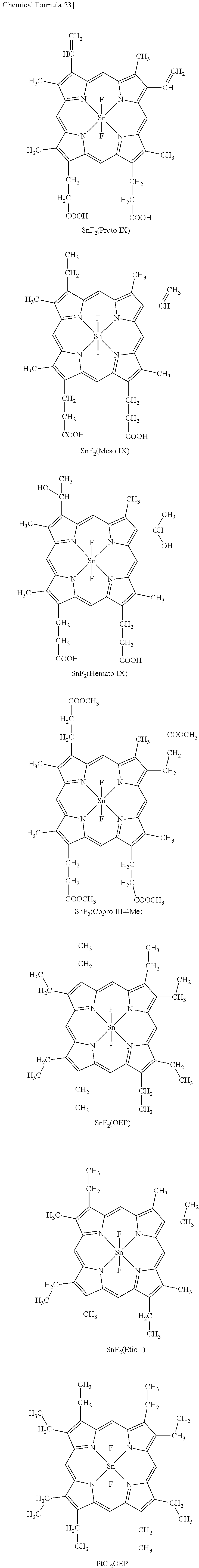

Alternatively, a heterocyclic compound having one or both of a π-electron rich heteroaromatic ring and a π-electron deficient heteroaromatic ring that is represented by the following structural formulae and typified by 2-(biphenyl-4-yl)-4,6-bis(12-phenylindolo[2,3-a]carbazol-11-yl)-1,3,5-triazine (abbreviation: PIC-TRZ), 9-(4,6-diphenyl-1,3,5-triazin-2-yl)-9′-phenyl-9H,9′H-3,3′-bicarbazole (abbreviation: PCCzTzn), 2-{4-[3-(N-phenyl-9H-carbazol-3-yl)-9H-carbazol-9-yl]phenyl}-4,6-diphenyl-1,3,5-triazine (abbreviation: PCCzPTzn), 2-[4-(10H-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DPS), or 10-phenyl-10H,10′H-spiro[acridin-9,9′-anthracen]-10′-one (abbreviation: ACRSA) can be used. These heterocyclic compounds are preferable because of having both a high electron-transport property and a high hole-transport property owing to the π-electron rich heteroaromatic ring and the π-electron deficient heteroaromatic ring. Among skeletons having a π-electron deficient heteroaromatic ring, a pyridine skeleton, a diazine skeleton (a pyrimidine skeleton, a pyrazine skeleton, and a pyridazine skeleton), and a triazine skeleton are particularly preferable because of their high stability and reliability. In particular, a benzofuropyrimidine skeleton, a benzothienopyrimidine skeleton, a benzofuropyrazine skeleton, and a benzothienopyrazine skeleton are preferable because of their high acceptor property and reliability. Among skeletons having a π-electron rich heteroaromatic ring, an acridine skeleton, a phenoxazine skeleton, a phenothiazine skeleton, a furan skeleton, a thiophene skeleton, and a pyrrole skeleton have high stability and reliability; therefore, at least one of these skeletons is preferably included. Note that a dibenzofuran skeleton and a dibenzothiophene skeleton are preferable as the furan skeleton and the thiophene skeleton, respectively. As the pyrrole skeleton, an indole skeleton, a carbazole skeleton, an indolocarbazole skeleton, a bicarbazole skeleton, and a 3-(9-phenyl-9H-carbazol-3-yl)-9H-carbazole skeleton are particularly preferable. Note that a substance in which a π-electron rich heteroaromatic ring and a π-electron deficient heteroaromatic ring are directly bonded to each other is particularly preferable because the electron-donating property of the π-electron rich heteroaromatic ring and the electron-accepting property of the π-electron deficient heteroaromatic ring are both increased and the energy difference between the S1 level and the T1 level becomes small, and thus thermally activated delayed fluorescence can be obtained efficiently. Note that an aromatic ring to which an electron-withdrawing group such as a cyano group is bonded may be used instead of the π-electron deficient heteroaromatic ring. As a π-electron rich skeleton, an aromatic amine skeleton, a phenazine skeleton, or the like can be used. As a π-electron deficient skeleton, a xanthene skeleton, a thioxanthene dioxide skeleton, an oxadiazole skeleton, a triazole skeleton, an imidazole skeleton, an anthraquinone skeleton, a boron-containing skeleton typified by phenylborane or boranthrene, an aromatic ring or a heteroaromatic ring having a nitrile group or a cyano group typified by benzonitrile or cyanobenzene, a carbonyl skeleton typified by benzophenone, a phosphine oxide skeleton, a sulfone skeleton, or the like can be used. As described above, a π-electron deficient skeleton and a π-electron rich skeleton can be used instead of at least one of the π-electron deficient heteroaromatic ring and the π-electron rich heteroaromatic ring.

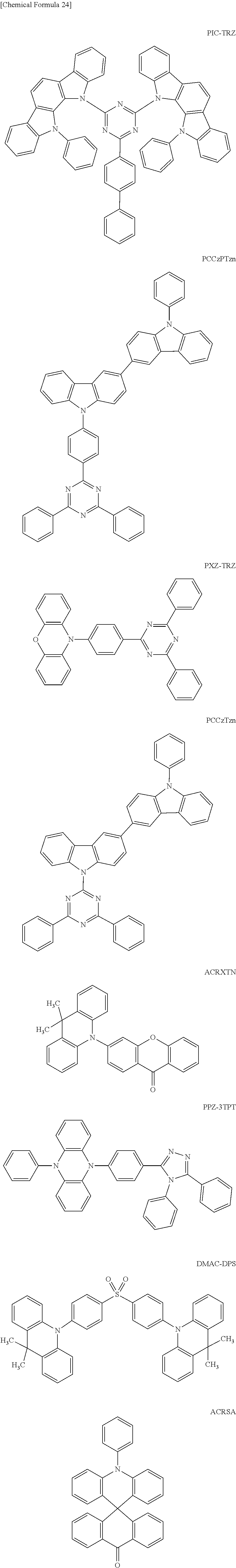

Note that the TADF material is a material that has a small difference between the S1 level and the T1 level and has a function of converting triplet excitation energy into singlet excitation energy by reverse intersystem crossing. Thus, it is possible to upconvert triplet excitation energy into singlet excitation energy (reverse intersystem crossing) using a little thermal energy and to efficiently generate a singlet excited state. In addition, the triplet excitation energy can be converted into light emission.

An exciplex whose excited state is formed by two kinds of substances has an extremely small difference between the S1 level and the T1 level and has a function of a TADF material that can convert triplet excitation energy into singlet excitation energy.

Note that a phosphorescent spectrum observed at low temperatures (e.g., 77 K to 10 K) is used for an index of the T1 level. When the level of energy with a wavelength of the line obtained by extrapolating a tangent to the fluorescent spectrum at a tail on the short wavelength side is the S1 level and the level of energy with a wavelength of the line obtained by extrapolating a tangent to the phosphorescent spectrum at a tail on the short wavelength side is the T1 level, the difference between S1 and T1 of the TADF material is preferably less than or equal to 0.3 eV, further preferably less than or equal to 0.2 eV.

When the TADF material is used as a light-emitting substance, the S1 level of the host material is preferably higher than the S1 level of the TADF material. In addition, the T1 level of the host material is preferably higher than the T1 level of the TADF material.

As the host material in the light-emitting layer, various carrier-transport materials such as a material having an electron-transport property, a material having a hole-transport property, and the TADF material can be used.

The material having a hole-transport property is preferably an organic compound having an amine skeleton or a π-electron rich heteroaromatic ring skeleton. Examples include a compound having an aromatic amine skeleton, such as 4,4′-bis[N-(1-naphthyl)-N-phenylamino]biphenyl (abbreviation: NPB), N,N-bis(3-methylphenyl)-N,N′-diphenyl-[1,1′-biphenyl]-4,4′-diamine (abbreviation: TPD), 4,4′-bis[N-(spiro-9,9′-bifluoren-2-yl)-N-phenylamino]biphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-(9-phenyl-9H-carbazol-3-yl)triphenylamine (abbreviation: PCBAIBP), 4,4′-diphenyl-4″-(9-phenyl-9H-carbazol-3-yl)triphenylamine (abbreviation: PCBBiIBP), 4-(1-naphthyl)-4′-(9-phenyl-9H-carbazol-3-yl)triphenylamine (abbreviation: PCBANB), 4,4′-di(i-naphthyl)-4″-(9-phenyl-9H-carbazol-3-yl)triphenylamine (abbreviation: PCBNBB), 9,9-dimethyl-N-phenyl-N-[4-(9-phenyl-9H-carbazol-3-yl)phenyl]fluoren-2-amine (abbreviation: PCBAF), or N-phenyl-N-[4-(9-phenyl-9H-carbazol-3-yl)phenyl]-9,9′-spirobi[9H-fluoren]-2-amine (abbreviation: PCBASF), a compound having a carbazole skeleton, such as 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), or 3,3′-bis(9-phenyl-9H-carbazole) (abbreviation: PCCP), a compound having a thiophene skeleton, such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), or 4-[4-(9-phenyl-9H-fluoren-9-yl)phenyl]-6-phenyldibenzothiophene (abbreviation: DBTFLP-IV), and a compound having a furan skeleton, such as 4,4′,4″-(benzene-1,3,5-triyl)tri(dibenzofuran) (abbreviation: DBF3P-II) or 4-{3-[3-(9-phenyl-9H-fluoren-9-yl)phenyl]phenyl}dibenzofuran (abbreviation: mmDBFFLBi-II). Among the above, the compound having an aromatic amine skeleton and the compound having a carbazole skeleton are preferable because these have favorable reliability, have high hole-transport properties, and contribute to a reduction in driving voltage.

As the material having an electron-transport property, for example, a metal complex such as bis(10-hydroxybenzo[h]quinolinato)beryllium(II) (abbreviation: BeBq₂), bis(2-methyl-8-quinolinolato)(4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), or bis[2-(2-benzothiazolyl)phenolato]zinc(II) (abbreviation: ZnBTZ); or an organic compound having a π-electron deficient heteroaromatic ring skeleton is preferable. Examples of the organic compound having a π-electron deficient heteroaromatic ring skeleton include a heterocyclic compound having a polyazole skeleton, such as 2-(4-biphenylyl)-5-(4-tert-butylphenyl)-1,3,4-oxadiazole (abbreviation: PBD), 3-(4-biphenylyl)-4-phenyl-5-(4-tert-butylphenyl)-1,2,4-triazole (abbreviation: TAZ), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: COIl), 2,2′,2″-(1,3,5-benzenetriyl)tris(1-phenyl-1H-benzimidazole) (abbreviation: TPBI), or 2-[3-(dibenzothiophen-4-yl)phenyl]-1-phenyl-1H-benzimidazole (abbreviation: mDBTBIm-II), a heterocyclic compound having a diazine skeleton, such as 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 4,6-bis[3-(phenanthren-9-yl)phenyl]pyrimidine (abbreviation: 4,6mPnP2Pm), 4,6-bis[3-(4-dibenzothienyl)phenyl]pyrimidine (abbreviation: 4,6mDBTP2Pm-II), or 2,8-bis[3-(dibenzothiophen-4-yl)phenyl]-benzo[h]quinazoline (abbreviation: 4,8mDBtP2Bqn), and a heterocyclic compound having a pyridine skeleton, such as 3,5-bis[3-(9H-carbazol-9-yl)phenyl]pyridine (abbreviation: 35DCzPPy) or 1,3,5-tri[3-(3-pyridyl)phenyl]benzene (abbreviation: TmPyPB). Among the above, the heterocyclic compound having a diazine skeleton and the heterocyclic compound having a pyridine skeleton have favorable reliability and thus are preferable. In particular, the heterocyclic compound having a diazine (e.g., pyrimidine or pyrazine) skeleton has a high electron-transport property and contributes to a reduction in driving voltage. Note that the organic compounds described in Embodiment 1 are organic compounds having electron-transport properties and thus can be suitably used as a host material in a light-emitting layer.

As the TADF material that can be used as the host material, the above-mentioned materials given as TADF materials can also be used. When the TADF material is used as the host material, triplet excitation energy generated in the TADF material is converted into singlet excitation energy by reverse intersystem crossing and transferred to the light-emitting substance, whereby the emission efficiency of the light-emitting device can be increased. At this time, the TADF material functions as an energy donor, and the light-emitting substance functions as an energy acceptor.

This is very effective in the case where the light-emitting substance is a fluorescent substance. In that case, the S1 level of the TADF material is preferably higher than the S1 level of the fluorescent substance in order to achieve high emission efficiency. Furthermore, the T1 level of the TADF material is preferably higher than the S1 level of the fluorescent substance. Therefore, the T1 level of the TADF material is preferably higher than the T1 level of the fluorescent substance.

It is also preferable to use a TADF material that exhibits light emission overlapping with the wavelength of a lowest-energy-side absorption band of the fluorescent substance. This enables smooth transfer of excitation energy from the TADF material to the fluorescent substance and accordingly enables efficient light emission, which is preferable.

In order that singlet excitation energy is efficiently generated from the triplet excitation energy by reverse intersystem crossing, carrier recombination preferably occurs in the TADF material. It is also preferable that the triplet excitation energy generated in the TADF material not be transferred to the triplet excitation energy of the fluorescent substance. For that reason, the fluorescent substance preferably has a protective group around a luminophore (a skeleton that causes light emission) of the fluorescent substance. As the protective group, a substituent having no 7t bond and saturated hydrocarbon are preferably used. Specific examples include an alkyl group having 3 to 10 carbon atoms, a substituted or unsubstituted cycloalkyl group having 3 to 10 carbon atoms, and a trialkylsilyl group having 3 to 10 carbon atoms. It is further preferable that the fluorescent substance have a plurality of protective groups. The substituent having no 7t bond has a poor carrier-transport property; thus, the TADF material and the luminophore of the fluorescent substance can be made away from each other with little influence on carrier transportation or carrier recombination. Here, the luminophore refers to an atomic group (skeleton) that causes light emission in a fluorescent substance. The luminophore is preferably a skeleton having a π bond, further preferably includes an aromatic ring, and still further preferably includes a condensed aromatic ring or a condensed heteroaromatic ring. Examples of the condensed aromatic ring or the condensed heteroaromatic ring include a phenanthrene skeleton, a stilbene skeleton, an acridone skeleton, a phenoxazine skeleton, and a phenothiazine skeleton. Specifically, a fluorescent substance having any of a naphthalene skeleton, an anthracene skeleton, a fluorene skeleton, a chrysene skeleton, a triphenylene skeleton, a tetracene skeleton, a pyrene skeleton, a perylene skeleton, a coumarin skeleton, a quinacridone skeleton, and a naphthobisbenzofuran skeleton is preferable because of its high fluorescence quantum yield.

In the case where a fluorescent substance is used as the light-emitting substance, a material having an anthracene skeleton is suitable for the host material. The use of a substance having an anthracene skeleton as a host material for a fluorescent substance makes it possible to achieve a light-emitting layer with favorable emission efficiency and durability. As the substance having an anthracene skeleton that is used as the host material, a substance having a diphenylanthracene skeleton, in particular, a substance having a 9,10-diphenylanthracene skeleton, is preferable because of its chemical stability. The host material preferably has a carbazole skeleton because the hole-injection and hole-transport properties are improved; further preferably, the host material has a benzocarbazole skeleton in which a benzene ring is further condensed to carbazole because the HOMO level thereof is shallower than that of carbazole by approximately 0.1 eV and thus holes enter the host material easily. In particular, the host material having a dibenzocarbazole skeleton is preferable because its HOMO level is shallower than that of carbazole by approximately 0.1 eV so that holes enter the host material easily, the hole-transport property is improved, and the heat resistance is increased. Accordingly, a substance that has both a 9,10-diphenylanthracene skeleton and a carbazole skeleton (or a benzocarbazole skeleton or a dibenzocarbazole skeleton) is further preferable as the host material. Note that in terms of the hole-injection and hole-transport properties described above, instead of a carbazole skeleton, a benzofluorene skeleton or a dibenzofluorene skeleton may be used. Examples of such a substance include 9-phenyl-3-[4-(10-phenyl-9-anthryl)phenyl]-9H-carbazole (abbreviation: PCzPA), 3-[4-(1-naphthyl)-phenyl]-9-phenyl-9H-carbazole (abbreviation: PCPN), 9-[4-(10-phenyl-9-anthracenyl)phenyl]-9H-carbazole (abbreviation: CzPA), 7-[4-(10-phenyl-9-anthryl)phenyl]-7H-dibenzo[c,g]carbazole (abbreviation: cgDBCzPA), 6-[3-(9,10-diphenyl-2-anthryl)phenyl]-benzo[b]naphtho[1,2-d]furan (abbreviation: 2mBnfPPA), 9-phenyl-10-{4-(9-phenyl-9H-fluoren-9-yl)biphenyl-4′-yl}anthracene (abbreviation: FLPPA), and 9-(1-naphthyl)-10-[4-(2-naphthyl)phenyl]anthracene (abbreviation: aN-PNPAnth). In particular, CzPA, cgDBCzPA, 2mBnfPPA, and PCzPA are preferably selected because they exhibit favorable characteristics.

Note that a host material may be a material of a mixture of a plurality of kinds of substances; in the case of using a mixed host material, it is preferable to mix a material having an electron-transport property with a material having a hole-transport property. When the material having an electron-transport property is mixed with the material having a hole-transport property, the transport property of the light-emitting layer 113 can be easily adjusted and a recombination region can be easily controlled. The weight ratio of the content of the material having a hole-transport property to the content of the material having an electron-transport property may be 1:19 to 19:1. Note that the organic compounds described in Embodiment 1 can be suitably used as the material having an electron-transport property in the mixed host material. Mixing of the material having an electron-transport property with the material having a hole-transport property may be conducted by co-evaporation or by evaporation of samples mixed in advance (premixed). The organic compound described in Embodiment 1 is also suitable for the latter mixing.

Note that a phosphorescent substance can be used as part of the mixed material. When a fluorescent substance is used as the light-emitting substance, a phosphorescent substance can be used as an energy donor for supplying excitation energy to the fluorescent substance.

An exciplex may be formed by these mixed materials. A combination is preferably selected so as to form an exciplex that exhibits light emission overlapping with the wavelength of a lowest-energy-side absorption band of a light-emitting substance, because energy can be transferred smoothly and light emission can be efficiently obtained. The use of the structure is preferable because the driving voltage is also be reduced.

Note that at least one of the materials forming an exciplex may be a phosphorescent substance. In this case, triplet excitation energy can be efficiently converted into singlet excitation energy by reverse intersystem crossing.

A combination of a material having an electron-transport property and a material having a hole-transport property whose HOMO level is higher than or equal to the HOMO level of the material having an electron-transport property is preferable for forming an exciplex efficiently. In addition, the LUMO level of the material having a hole-transport property is preferably higher than or equal to the LUMO level of the material having an electron-transport property. Note that the LUMO levels and the HOMO levels of the materials can be derived from the electrochemical characteristics (the reduction potentials and the oxidation potentials) of the materials that are measured by cyclic voltammetry (CV).

Note that the formation of an exciplex can be confirmed by a phenomenon in which the emission spectrum of the mixed film in which the material having a hole-transport property and the material having an electron-transport property are mixed is shifted to the longer wavelength side than the emission spectrum of each of the materials (or has another peak on the longer wavelength side), observed by comparison of the emission spectrum of the material having a hole-transport property, the emission spectrum of the material having an electron-transport property, and the emission spectrum of the mixed film of these materials, for example. Alternatively, the formation of an exciplex can be confirmed by a difference in transient response, such as a phenomenon in which the transient photoluminescence (PL) lifetime of the mixed film has longer lifetime components or has a larger proportion of delayed components than that of each of the materials, observed by comparison of the transient PL of the material having a hole-transport property, the transient PL of the material having an electron-transport property, and the transient PL of the mixed film of these materials. The transient PL can be rephrased as transient electroluminescence (EL). That is, the formation of an exciplex can also be confirmed by a difference in transient response observed by comparison of the transient EL of the material having a hole-transport property, the transient EL of the material having an electron-transport property, and the transient EL of the mixed film of these materials.

The electron-transport layer 114 is a layer containing a substance having an electron-transport property. As the substance having an electron-transport property, it is possible to use any of the above-listed substances having electron-transport properties that can be used as the host material.

The electron mobility of the electron-transport layer 114 in the case where the square root of the electric field strength [V/cm] is 600 is preferably higher than or equal to 1×10⁻⁷ cm²/Vs and lower than or equal to 5×10⁻⁵ cm²/Vs. Lowering the electron-transport property of the electron-transport layer 114 enables control of the amount of electrons injected into the light-emitting layer and can prevent the light-emitting layer from having excess electrons. The electron-transport layer 114 preferably includes a material having an electron-transport property and an alkali metal, an alkaline earth metal, a compound thereof, or a complex thereof. It is particularly preferable that this structure be employed when the hole-injection layer is formed using a composite material that includes a material having a hole-transport property with a relatively deep HOMO level of −5.7 eV or higher and −5.4 eV or lower, in which case a long lifetime can be achieved. Here, the material having an electron-transport property preferably has a HOMO level of higher than or equal to −6.0 eV. The material having an electron-transport property is preferably an organic compound having an anthracene skeleton and is further preferably an organic compound having both an anthracene skeleton and a heterocyclic skeleton. The heterocyclic skeleton is preferably a nitrogen-containing five-membered ring skeleton or a nitrogen-containing six-membered ring skeleton, and particularly preferably a nitrogen-containing five-membered ring skeleton or a nitrogen-containing six-membered ring skeleton including two heteroatoms in the ring, such as a pyrazole ring, an imidazole ring, an oxazole ring, a thiazole ring, a pyrazine ring, a pyrimidine ring, or a pyridazine ring. In addition, it is preferable that the alkali metal itself, the alkaline earth metal itself, the compound thereof, and the complex thereof have an 8-hydroxyquinolinato structure. Specific examples include 8-hydroxyquinolinato-lithium (abbreviation: Liq) and 8-hydroxyquinolinato-sodium (abbreviation: Naq). In particular, a complex of a monovalent metal ion, especially a complex of lithium is preferable, and Liq is further preferable. Note that in the case where the 8-hydroxyquinolinato structure is included, a methyl-substituted product (e.g., a 2-methyl-substituted product or a 5-methyl-substituted product) thereof or the like can also be used. There is preferably a difference in the concentration (including 0) of the alkali metal itself, the alkaline earth metal itself, the compound thereof, or the complex thereof in the electron-transport layer in the thickness direction.

As the electron-injection layer 115, a layer containing an alkali metal, an alkaline earth metal, or a compound thereof typified by lithium fluoride (LiF), cesium fluoride (CsF), calcium fluoride (CaF₂), or 8-hydroxyquinolinato-lithium (abbreviation: Liq), may be provided between the electron-transport layer 114 and the second electrode 102. An electride or a layer that is formed using a substance having an electron-transport property and that includes an alkali metal, an alkaline earth metal, or a compound thereof can be used as the electron-injection layer 115. Examples of the electride include a substance in which electrons are added at high concentration to a mixed oxide of calcium and aluminum.

Note that as the electron-injection layer 115, it is possible to use a layer that contains a substance having an electron-transport property (preferably an organic compound having a bipyridine skeleton) and contains a fluoride of the alkali metal or the alkaline earth metal at a concentration higher than or equal to that at which the electron-injection layer 115 becomes in a microcrystalline state (50 wt % or higher). Since the layer has a low refractive index, a light-emitting device having more favorable external quantum efficiency can be provided.

Instead of the electron-injection layer 115, the charge-generation layer 116 may be provided (FIG. 1B). The charge-generation layer 116 refers to a layer capable of injecting holes into a layer in contact therewith on the cathode side and injecting electrons into a layer in contact therewith on the anode side when supplied with a potential. The charge-generation layer 116 includes at least a P-type layer 117. The P-type layer 117 is preferably formed using the composite materials given above as the material that can form the hole-injection layer 111. The P-type layer 117 may be formed by stacking a film containing the above acceptor material as a material included in the composite material and a film containing the above hole-transport material. When a potential is applied to the P-type layer 117, electrons are injected into the electron-transport layer 114 and holes are injected into the second electrode 102 that is a cathode; thus, the light-emitting device operates. Since the organic compound of one embodiment of the present invention has a low refractive index, using the organic compound for the P-type layer 117 enables the light-emitting device to have high external quantum efficiency.

Note that one or both of an electron-relay layer 118 and an electron-injection buffer layer 119 are preferably provided in the charge-generation layer 116 in addition to the P-type layer 117.

The electron-relay layer 118 contains at least a substance having an electron-transport property and has a function of preventing an interaction between the electron-injection buffer layer 119 and the P-type layer 117 to transfer electrons smoothly. The LUMO level of the substance having an electron-transport property contained in the electron-relay layer 118 is preferably between the LUMO level of an acceptor substance in the P-type layer 117 and the LUMO level of a substance contained in a layer of the electron-transport layer 114 in contact with the charge-generation layer 116. A specific energy level of the LUMO level of the substance having an electron-transport property used for the electron-relay layer 118 may be higher than or equal to −5.0 eV, preferably higher than or equal to −5.0 eV and lower than or equal to −3.0 eV. Note that as the substance having an electron-transport property used for the electron-relay layer 118, a phthalocyanine-based material or a metal complex having a metal-oxygen bond and an aromatic ligand is preferably used.

For the electron-injection buffer layer 119, a substance having a high electron-injection property typified by an alkali metal, an alkaline earth metal, a rare earth metal, or a compound thereof (an alkali metal compound (including an oxide typified by lithium oxide, a halide, and a carbonate typified by lithium carbonate or cesium carbonate), an alkaline earth metal compound (including an oxide, a halide, and a carbonate), or a rare earth metal compound (including an oxide, a halide, and a carbonate)), can be used.

In the case where the electron-injection buffer layer 119 is formed so as to contain the substance having an electron-transport property and a donor substance, an organic compound typified by tetrathianaphthacene (abbreviation: TTN), nickelocene, or decamethylnickelocene can be used as the donor substance, as well as an alkali metal, an alkaline earth metal, a rare earth metal, a compound thereof (an alkali metal compound (including an oxide typified by lithium oxide, a halide, and a carbonate typified by lithium carbonate or cesium carbonate), an alkaline earth metal compound (including an oxide, a halide, and a carbonate), or a rare earth metal compound (including an oxide, a halide, and a carbonate)). Note that as the substance having an electron-transport property, a material similar to the above-described material forming the electron-transport layer 114 can be used for the formation.

As a substance forming the second electrode 102, a metal, an alloy, an electrically conductive compound, or a mixture thereof having a low work function (specifically, 3.8 eV or less) or the like can be used. Specific examples of such a cathode material include elements belonging to Group 1 and Group 2 of the periodic table typified by alkali metals (typified by lithium (Li) and cesium (Cs)), magnesium (Mg), calcium (Ca), and strontium (Sr), alloys containing these (MgAg and AlLi), rare earth metals typified by europium (Eu) and ytterbium (Yb), alloys containing these rare earth metals, and the like can be given. However, when the electron-injection layer is provided between the second electrode 102 and the electron-transport layer, as the second electrode 102, a variety of conductive materials such as Al, Ag, ITO, or indium oxide-tin oxide containing silicon or silicon oxide can be used regardless of their work functions. Films of these conductive materials can be formed by a dry process such as a vacuum evaporation method or a sputtering method, an inkjet method, a spin coating method, or the like. Alternatively, the films may be formed by a wet process using a sol-gel method or a wet process using a paste of a metal material.

Various methods can be used as a method for forming the EL layer 103 regardless of whether it is a dry process or a wet process. For example, a vacuum evaporation method, a gravure printing method, an offset printing method, a screen printing method, an ink-jet method, a spin coating method, or the like may be used.

Different deposition methods may be used to form the electrodes or the layers described above.

The structure of the layers provided between the first electrode 101 and the second electrode 102 is not limited to the above structure. However, a structure is preferable in which a light-emitting region where holes and electrons recombine is provided at a position away from the first electrode 101 and the second electrode 102 so as to prevent quenching caused by the proximity of the light-emitting region and a metal used for electrodes and carrier-injection layers.

Furthermore, in order to inhibit energy transfer from an exciton generated in the light-emitting layer, it is preferable to form the hole-transport layer and the electron-transport layer that are in contact with the light-emitting layer 113, particularly a carrier-transport layer closer to the recombination region in the light-emitting layer 113, using the light-emitting material of the light-emitting layer or a substance having a wider band gap than the light-emitting material included in the light-emitting layer.

Next, an embodiment of a light-emitting device with a structure where a plurality of light-emitting units are stacked (also referred to as a stacked-type element or a tandem element) will be described with reference to FIG. 1C. This light-emitting device is a light-emitting device including a plurality of light-emitting units between an anode and a cathode. One light-emitting unit has substantially the same structure as that of the EL layer 103, which is illustrated in FIG. TA. In other words, the light-emitting device illustrated in FIG. 1C can be called a light-emitting device including a plurality of light-emitting units, and the light-emitting device illustrated in FIG. TA or FIG. 1B can be called a light-emitting device including one light-emitting unit. Note that the organic compound described in Embodiment 1 is included in at least any of the plurality of light-emitting units.

In FIG. 1C, a first light-emitting unit 511 and a second light-emitting unit 512 are stacked between an anode 501 and a cathode 502, and a charge-generation layer 513 is provided between the first light-emitting unit 511 and the second light-emitting unit 512. The anode 501 and the cathode 502 correspond, respectively, to the first electrode 101 and the second electrode 102 in FIG. 1A, and the same substance as what is given in the description for FIG. 1A can be used. Furthermore, the first light-emitting unit 511 and the second light-emitting unit 512 may have the same structure or different structures.

The charge-generation layer 513 has a function of injecting electrons into one of the light-emitting units and injecting holes into the other of the light-emitting units when a voltage is applied to the anode 501 and the cathode 502. That is, in FIG. 1C, any layer can be used as the charge-generation layer 513 as long as the layer injects electrons into the first light-emitting unit 511 and injects holes into the second light-emitting unit 512 in the case where a voltage is applied such that the potential of the anode is higher than the potential of the cathode.

The charge-generation layer 513 is preferably formed with a structure similar to that of the charge-generation layer 116 described with reference to FIG. 1B. A composite material of an organic compound and a metal oxide has an excellent carrier-injection property and an excellent carrier-transport property; thus, low-voltage driving and low-current driving can be achieved. Note that in the case where the anode-side surface of a light-emitting unit is in contact with the charge-generation layer 513, the charge-generation layer 513 can also serve as a hole-injection layer of the light-emitting unit; therefore, a hole-injection layer is not necessarily provided in the light-emitting unit.

In the case where the electron-injection buffer layer 119 is provided in the charge-generation layer 513, the electron-injection buffer layer 119 serves as an electron-injection layer in the light-emitting unit on the anode side; therefore, an electron-injection layer is not necessarily formed in the light-emitting unit on the anode side.

The light-emitting device having two light-emitting units is described with reference to FIG. 1C; however, the same can also be applied to a light-emitting device in which three or more light-emitting units are stacked. With a plurality of light-emitting units partitioned by the charge-generation layer 513 between a pair of electrodes as in the light-emitting device according to this embodiment, it is possible to provide a long-life device that can emit light with high luminance at a low current density. Moreover, a light-emitting apparatus that can be driven at a low voltage and has low power consumption can be achieved.

Furthermore, when emission colors of the light-emitting units are different, light emission of a desired color can be obtained from the light-emitting device as a whole. For example, in a light-emitting device having two light-emitting units, emission colors of red and green are obtained in the first light-emitting unit and an emission color of blue is obtained in the second light-emitting unit, whereby a light-emitting device that emits white light as the whole light-emitting device can be obtained.

The EL layer 103, the first light-emitting unit 511, the second light-emitting unit 512, the layers such as the charge-generation layer, or the electrodes that are described above can be formed by a method typified by an evaporation method (including a vacuum evaporation method), a droplet discharge method (also referred to as an ink-jet method), a coating method, or a gravure printing method. Those may include a low molecular material, a middle molecular material (including an oligomer and a dendrimer), or a high molecular material.

### Embodiment 3

In this embodiment, a light-emitting apparatus using the light-emitting device described in Embodiment 2 will be described.

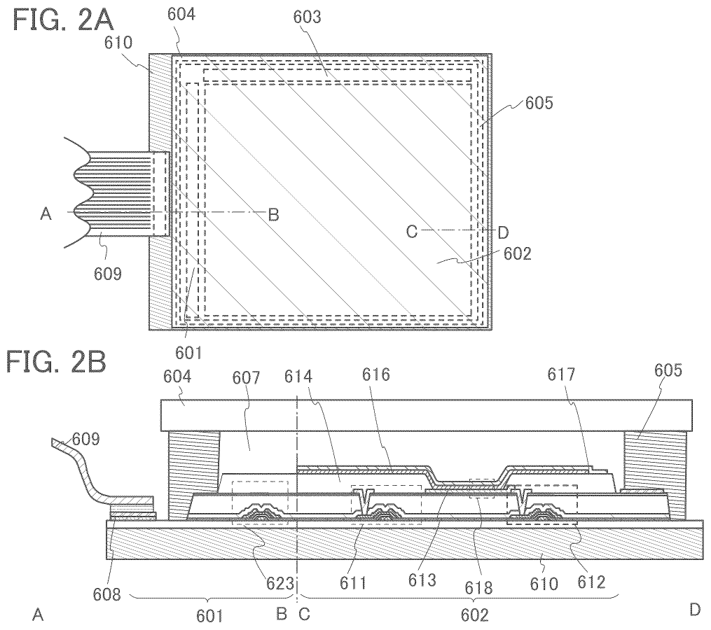

In this embodiment, a light-emitting apparatus fabricated using the light-emitting device described in Embodiment 2 will be described with reference to FIG. 2. Note that FIG. 2A is a top view illustrating the light-emitting apparatus, and FIG. 2B is a cross-sectional view taken along A-B and C-D in FIG. 2A. This light-emitting apparatus includes a driver circuit portion (source line driver circuit) 601, a pixel portion 602, and a driver circuit portion (gate line driver circuit) 603, which are for controlling light emission of a light-emitting device and are illustrated with dotted lines. Furthermore, 604 denotes a sealing substrate, 605 denotes a sealant, and the inside surrounded by the sealant 605 is a space 607.

Note that a lead wiring 608 is a wiring for transmitting signals to be input to the source line driver circuit 601 and the gate line driver circuit 603 and receiving a video signal, a clock signal, a start signal, a reset signal, and the like from an FPC (flexible printed circuit) 609 serving as an external input terminal. Although only the FPC is illustrated here, a printed wiring board (PWB) may be attached to this FPC. The light-emitting apparatus in this specification includes not only the light-emitting apparatus itself but also the apparatus provided with the FPC or the PWB.

Next, a cross-sectional structure will be described with reference to FIG. 2B. The driver circuit portion and the pixel portion are formed over an element substrate 610. Here, the source line driver circuit 601, which is the driver circuit portion, and one pixel of the pixel portion 602 are illustrated.

The element substrate 610 may be fabricated using a substrate containing glass, quartz, an organic resin, a metal, an alloy, a semiconductor, or the like, or a plastic substrate formed of FRP (Fiber Reinforced Plastic), PVF (polyvinyl fluoride), polyester, an acrylic resin, or the like.

There is no particular limitation on the structure of transistors used in pixels and driver circuits. For example, an inverted staggered transistor or a staggered transistor may be used. Furthermore, top-gate transistors or bottom-gate transistors may be used. There is no particular limitation on a semiconductor material used for the transistors, and for example, silicon, germanium, silicon carbide, gallium nitride, or the like can be used. Alternatively, an oxide semiconductor containing at least one of indium, gallium, and zinc, such as In—Ga—Zn-based metal oxide, may be used.

There is no particular limitation on the crystallinity of a semiconductor material used for the transistors, and any of an amorphous semiconductor or a semiconductor having crystallinity (a microcrystalline semiconductor, a polycrystalline semiconductor, a single-crystal semiconductor, and a semiconductor partly including crystal regions) may be used. A semiconductor having crystallinity is preferably used, in which case deterioration of the transistor characteristics can be suppressed.

Here, an oxide semiconductor is preferably used for semiconductor devices typified by the transistors provided in the pixels and driver circuits and transistors used for touch sensors described later, and the like. In particular, an oxide semiconductor having a wider band gap than silicon is preferably used. The use of an oxide semiconductor having a wider band gap than silicon can reduce the off-state current of the transistors.

The oxide semiconductor preferably contains at least indium (In) or zinc (Zn). Further preferably, the oxide semiconductor contains an oxide represented by an In-M-Zn-based oxide (M represents a metal typified by Al, Ti, Ga, Ge, Y, Zr, Sn, La, Ce, or Hf).

As a semiconductor layer, it is particularly preferable to use an oxide semiconductor film including a plurality of crystal parts whose c-axes are aligned perpendicular to a surface on which the semiconductor layer is formed or the top surface of the semiconductor layer and in which the adjacent crystal parts have no grain boundary.

The use of such a material for the semiconductor layer makes it possible to achieve a highly reliable transistor in which a change in the electrical characteristics is reduced.

Charge accumulated in a capacitor through a transistor including the above-described semiconductor layer can be retained for a long time because of the low off-state current of the transistor. The use of such a transistor in pixels allows a driver circuit to stop while the gray level of an image displayed on each display region is maintained. As a result, an electronic device with significantly reduced power consumption can be achieved.

For stable characteristics of the transistor, a base film is preferably provided. The base film can be formed to be a single-layer or a stacked-layer using an inorganic insulating film such as a silicon oxide film, a silicon nitride film, a silicon oxynitride film, or a silicon nitride oxide film. The base film can be formed by a sputtering method, a CVD (Chemical Vapor Deposition) method (e.g., a plasma CVD method, a thermal CVD method, or an MOCVD (Metal Organic CVD) method), an ALD (Atomic Layer Deposition) method, a coating method, a printing method, or the like. Note that the base film is not necessarily provided when not needed.

Note that an FET 623 is illustrated as a transistor formed in the driver circuit portion 601. The driver circuit can be formed using various circuits such as a CMOS circuit, a PMOS circuit, and an NMOS circuit. Although a driver-integrated type in which the driver circuit is formed over the substrate is described in this embodiment, the driver circuit is not necessarily formed over the substrate and can be formed outside.

The pixel portion 602 is formed with a plurality of pixels including a switching FET 611, a current control FET 612, and a first electrode 613 electrically connected to a drain of the current control FET 612; however, without being limited thereto, a pixel portion in which three or more FETs and a capacitor are combined may be employed.

Note that an insulator 614 is formed to cover an end portion of the first electrode 613. The insulator 614 can be formed using a positive photosensitive acrylic resin film here.

In order to improve the coverage with an EL layer or the like to be formed later, the insulator 614 is formed so as to have a curved surface with curvature at its upper end portion or lower end portion. For example, in the case where a positive photosensitive acrylic resin is used as a material for the insulator 614, only the upper end portion of the insulator 614 preferably has a curved surface with a curvature radius (0.2 μm to 3 μm). As the insulator 614, either a negative photosensitive resin or a positive photosensitive resin can be used.

An EL layer 616 and a second electrode 617 are formed over the first electrode 613. Here, as a material used for the first electrode 613 functioning as an anode, a material with a high work function is desirably used. For example, a single-layer film of an ITO film, an indium tin oxide film containing silicon, an indium oxide film containing zinc oxide at 2 wt % to 20 wt %, a titanium nitride film, a chromium film, a tungsten film, a Zn film, a Pt film, or the like, a stacked layer of titanium nitride film and a film containing aluminum as its main component, a three-layer structure of a titanium nitride film, a film containing aluminum as its main component, and a titanium nitride film, or the like can be used. Note that the stacked-layer structure achieves low wiring resistance, a favorable ohmic contact, and a function as an anode.

The EL layer 616 is formed by any of a variety of methods typified by an evaporation method using an evaporation mask, an inkjet method, and a spin coating method. The EL layer 616 has the structure described in Embodiment 2. Alternatively, a material included in the EL layer 616 may be a low molecular compound or a high molecular compound (including an oligomer or a dendrimer).

As a material used for the second electrode 617, which is formed over the EL layer 616 and functions as a cathode, a material with a low work function (e.g., Al, Mg, Li, Ca, or an alloy or a compound thereof (e.g., MgAg, MgIn, or AlLi)) is preferably used. Note that in the case where light generated in the EL layer 616 passes through the second electrode 617, it is preferable to use, for the second electrode 617, a stacked layer of a thin metal film and a transparent conductive film (e.g., ITO, indium oxide containing zinc oxide at 2 wt % to 20 wt %, indium tin oxide containing silicon, or zinc oxide (ZnO)).

Note that a light-emitting device is formed with the first electrode 613, the EL layer 616, and the second electrode 617. The light-emitting device is the light-emitting device described in Embodiment 2. A plurality of light-emitting devices are formed in the pixel portion, and the light-emitting apparatus of this embodiment may include both the light-emitting device described in Embodiment 2 and a light-emitting device having a different structure.

The sealing substrate 604 and the element substrate 610 are attached to each other using the sealant 605, so that a structure is employed in which a light-emitting device 618 is provided in the space 607 surrounded by the element substrate 610, the sealing substrate 604, and the sealant 605. The space 607 is filled with a filler; it is filled with an inert gas (e.g., nitrogen or argon) in some cases, and filled with the sealant in some cases. The structure of the sealing substrate in which a recessed portion is formed and a desiccant is provided is preferable because deterioration due to the influence of moisture can be inhibited.

Note that an epoxy-based resin or glass frit is preferably used for the sealant 605. Furthermore, these materials are preferably materials that transmit moisture or oxygen as little as possible. As the material used for the sealing substrate 604, in addition to a glass substrate and a quartz substrate, a plastic substrate formed of FRP (Fiber Reinforced Plastics), PVF (polyvinyl fluoride), polyester, an acrylic resin, or the like can be used.

Although not illustrated in FIG. 2, a protective film may be provided over the second electrode. The protective film may be formed using an organic resin film or an inorganic insulating film. The protective film may be formed so as to cover an exposed portion of the sealant 605. The protective film may be provided so as to cover surfaces and side surfaces of the pair of substrates and exposed side surfaces of a sealing layer and an insulating layer.

For the protective film, a material that is less likely to transmit an impurity typified by water can be used. Thus, diffusion of the impurity from the outside into the inside can be effectively inhibited.

As a material included in the protective film, an oxide, a nitride, a fluoride, a sulfide, a ternary compound, a metal, a polymer, or the like can be used; for example, it is possible to use a material containing aluminum oxide, hafnium oxide, hafnium silicate, lanthanum oxide, silicon oxide, strontium titanate, tantalum oxide, titanium oxide, zinc oxide, niobium oxide, zirconium oxide, tin oxide, yttrium oxide, cerium oxide, scandium oxide, erbium oxide, vanadium oxide, indium oxide, or the like; a material containing aluminum nitride, hafnium nitride, silicon nitride, tantalum nitride, titanium nitride, niobium nitride, molybdenum nitride, zirconium nitride, gallium nitride, or the like; or a material containing a nitride containing titanium and aluminum, an oxide containing titanium and aluminum, an oxide containing aluminum and zinc, a sulfide containing manganese and zinc, a sulfide containing cerium and strontium, an oxide containing erbium and aluminum, an oxide containing yttrium and zirconium, or the like.

The protective film is preferably formed using a deposition method that enables favorable step coverage. One such method is an atomic layer deposition (ALD) method. A material that can be formed by an ALD method is preferably used for the protective film. With the use of an ALD method, a dense protective film with reduced defects such as cracks and pinholes or with a uniform thickness can be formed. Furthermore, damage caused to a process member in forming the protective film can be reduced.

By an ALD method, a uniform protective film with few defects can be formed even on a surface with a complex uneven shape or upper, side, and lower surfaces of a touch panel.

As described above, the light-emitting apparatus fabricated using the light-emitting device described in Embodiment 2 can be obtained.

For the light-emitting apparatus in this embodiment, the light-emitting device described in Embodiment 2 is used and thus a light-emitting apparatus having favorable characteristics can be obtained. Specifically, since the light-emitting device described in Embodiment 2 has high emission efficiency, the light-emitting apparatus with low power consumption can be obtained.

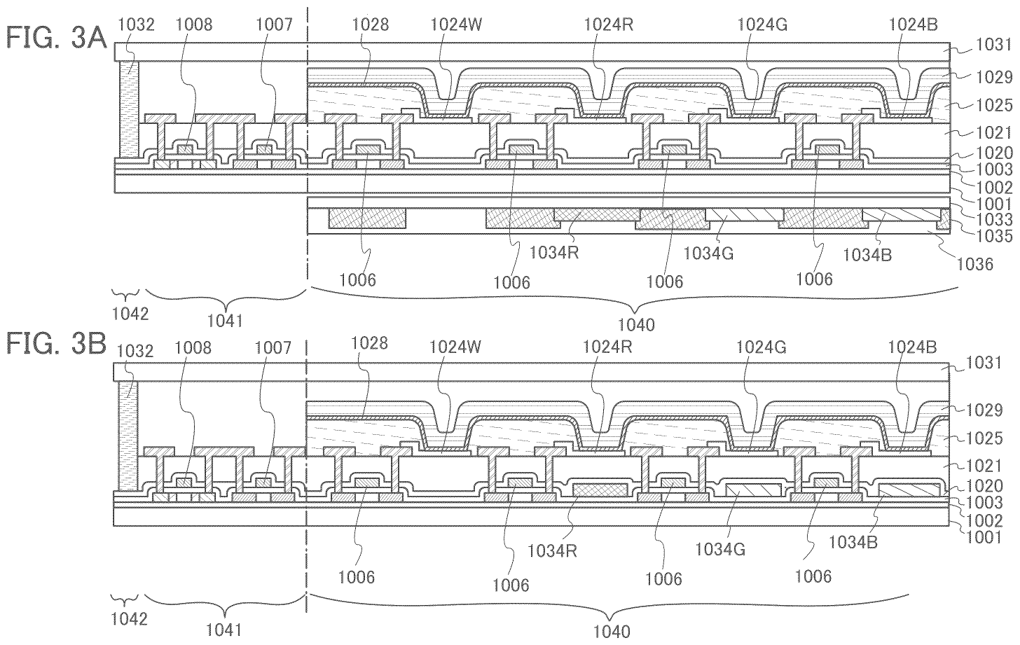

FIG. 3 illustrates examples of a light-emitting apparatus in which full color display is achieved by formation of a light-emitting device exhibiting white light emission and provision of coloring layers (color filters) and the like. FIG. 3A illustrates a substrate 1001, a base insulating film 1002, a gate insulating film 1003, gate electrodes 1006, 1007, and 1008, a first interlayer insulating film 1020, a second interlayer insulating film 1021, a peripheral portion 1042, a pixel portion 1040, a driver circuit portion 1041, first electrodes 1024W, 1024R, 1024G, and 1024B of the light-emitting devices, a partition 1025, an EL layer 1028, a second electrode 1029 of the light-emitting devices, a sealing substrate 1031, a sealant 1032, and the like.

In FIG. 3A, coloring layers (a red coloring layer 1034R, a green coloring layer 1034G, and a blue coloring layer 1034B) are provided on a transparent base material 1033. A black matrix 1035 may be additionally provided. The transparent base material 1033 provided with the coloring layers and the black matrix is positioned and fixed to the substrate 1001. Note that the coloring layers and the black matrix 1035 are covered with an overcoat layer 1036. In FIG. 3A, a light-emitting layer from which light is emitted to the outside without passing through the coloring layer and light-emitting layers from which light is emitted to the outside, passing through the coloring layers of the respective colors are shown. Since light that does not pass through the coloring layer is white and light that passes through the coloring layer is red, green, or blue, an image can be expressed by pixels of the four colors.

FIG. 3B shows an example in which the coloring layers (the red coloring layer 1034R, the green coloring layer 1034G, and the blue coloring layer 1034B) are formed between the gate insulating film 1003 and the first interlayer insulating film 1020. The coloring layers may be provided between the substrate 1001 and the sealing substrate 1031 in this manner.

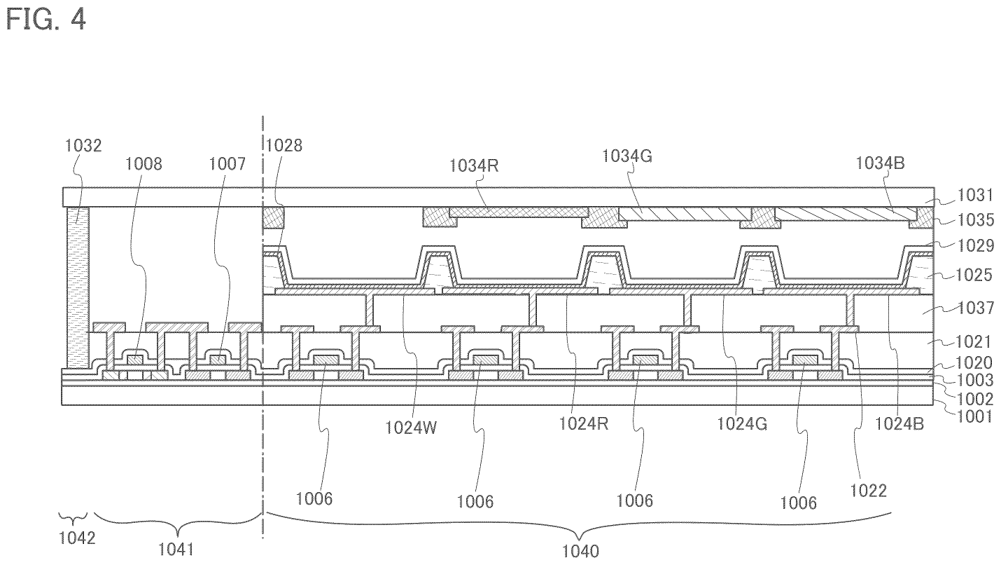

The above-described light-emitting apparatus is a light-emitting apparatus having a structure in which light is extracted to the substrate 1001 side where the FETs are formed (a bottom-emission type), but may be a light-emitting apparatus having a structure in which light emission is extracted to the sealing substrate 1031 side (a top-emission type). FIG. 4 shows a cross-sectional view of a top-emission light-emitting apparatus. In this case, a substrate that does not transmit light can be used as the substrate 1001. The top-emission light-emitting apparatus is formed in a manner similar to that of the bottom-emission light-emitting apparatus until an electrode 1022 which connects the FET and the anode of the light-emitting device is formed. Then, a third interlayer insulating film 1037 is formed to cover the electrode 1022. This insulating film may have a planarization function. The third interlayer insulating film 1037 can be formed using a material similar to that for the second interlayer insulating film or using any other known materials.

The first electrodes 1024W, 1024R, 1024G, and 1024B of the light-emitting devices are each an anode here, but may each be a cathode. Furthermore, in the case of the top-emission light-emitting apparatus illustrated in FIG. 4, the first electrodes are preferably reflective electrodes. The structure of the EL layer 1028 is such a structure as that of the EL layer 103 described in Embodiment 2, and an element structure with which white light emission can be obtained.

In the case of such a top-emission structure as in FIG. 4, sealing can be performed with the sealing substrate 1031 on which the coloring layers (the red coloring layer 1034R, the green coloring layer 1034G, and the blue coloring layer 1034B) are provided. The sealing substrate 1031 may be provided with the black matrix 1035 which is positioned between pixels. The coloring layers (the red coloring layer 1034R, the green coloring layer 1034G, and the blue coloring layer 1034B) and the black matrix may be covered with the overcoat layer 1036. Note that a light-transmitting substrate is used as the sealing substrate 1031. Although an example in which full color display is performed using four colors of red, green, blue, and white is shown here, there is no particular limitation and full color display may be performed using four colors of red, yellow, green, and blue or three colors of red, green, and blue.

In the top-emission-type light-emitting apparatus, a microcavity structure can be favorably employed. A light-emitting device with a microcavity structure can be obtained with the use of a reflective electrode as the first electrode and a semi-transmissive and semi-reflective electrode as the second electrode. The light-emitting device with a microcavity structure includes at least an EL layer between the reflective electrode and the semi-transmissive and semi-reflective electrode, which includes at least a light-emitting layer serving as a light-emitting region.

Note that the reflective electrode is a film having a visible light reflectivity of 40% to 100%, preferably 70% to 100%, and a resistivity of 1×10⁻² Ωcm or lower. In addition, the semi-transmissive and semi-reflective electrode is a film having a visible light reflectivity of 20% to 80%, preferably 40% to 70%, and a resistivity of 1×10⁻² Ωcm or lower.

Light emitted from the light-emitting layer included in the EL layer is reflected and resonated by the reflective electrode and the semi-transmissive and semi-reflective electrode.

In the light-emitting device, by changing thicknesses of the transparent conductive film, the above-described composite material, the carrier-transport material, and the like, the optical path length between the reflective electrode and the semi-transmissive and semi-reflective electrode can be changed. Thus, light with a wavelength that is resonated between the reflective electrode and the semi-transmissive and semi-reflective electrode can be intensified while light with a wavelength that is not resonated therebetween can be attenuated.

Note that light that is reflected back by the reflective electrode (first reflected light) considerably interferes with light that directly enters the semi-transmissive and semi-reflective electrode from the light-emitting layer (first incident light); therefore, the optical path length between the reflective electrode and the light-emitting layer is preferably adjusted to (2n−1)λ/4 (n is a natural number of 1 or larger and λ is a wavelength of light emission to be amplified). By adjusting the optical path length, the phases of the first reflected light and the first incident light can be aligned with each other and the light emitted from the light-emitting layer can be further amplified.

Note that in the above structure, the EL layer may include a plurality of light-emitting layers or may include a single light-emitting layer; for example, in combination with the structure of the above-described tandem light-emitting device, a plurality of EL layers each including a single or a plurality of light-emitting layer(s) may be provided in one light-emitting device with a charge-generation layer interposed between the EL layers.

With the microcavity structure, emission intensity with a specific wavelength in the front direction can be increased, whereby power consumption can be reduced. Note that in the case of a light-emitting apparatus which displays images with subpixels of four colors, red, yellow, green, and blue, the light-emitting apparatus can have favorable characteristics because a microcavity structure suitable for wavelengths of the corresponding color is employed in each subpixel, in addition to the effect of an improvement in luminance owing to yellow light emission.

For the light-emitting apparatus in this embodiment, the light-emitting device described in Embodiment 2 is used and thus a light-emitting apparatus having favorable characteristics can be obtained. Specifically, since the light-emitting device described in Embodiment 2 has high emission efficiency, the light-emitting apparatus with low power consumption can be obtained.

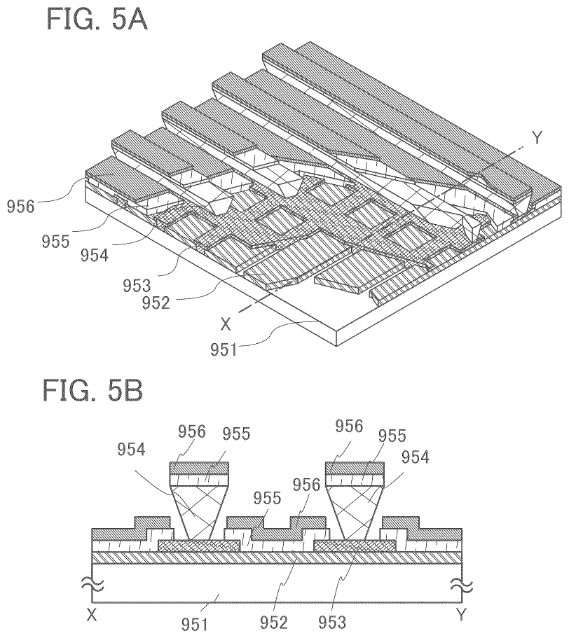

The active matrix light-emitting apparatus is described above, whereas a passive matrix light-emitting apparatus is described below. FIG. 5 illustrates a passive matrix light-emitting apparatus fabricated using the present invention. Note that FIG. 5A is a perspective view illustrating the light-emitting apparatus, and FIG. 5B is a cross-sectional view taken along X-Y in FIG. 5A. In FIG. 5, over a substrate 951, an EL layer 955 is provided between an electrode 952 and an electrode 956. An end portion of the electrode 952 is covered with an insulating layer 953. A partition layer 954 is provided over the insulating layer 953. Sidewalls of the partition layer 954 are aslope such that the distance between one sidewall and the other sidewall is gradually narrowed toward the surface of the substrate. That is, a cross section in the short side direction of the partition layer 954 is a trapezoidal shape, and the lower side (the side facing the same direction as the plane direction of the insulating layer 953 and touching the insulating layer 953) is shorter than the upper side (the side facing the same direction as the plane direction of the insulating layer 953, and not touching the insulating layer 953). By providing the partition layer 954 in this manner, defects of the light-emitting device due to static charge or the like can be prevented. The passive-matrix light-emitting apparatus also uses the light-emitting device described in Embodiment 2; thus, the light-emitting apparatus can have favorable reliability or low power consumption.

Since many minute light-emitting devices arranged in a matrix can each be controlled in the light-emitting apparatus described above, the light-emitting apparatus can be suitably used as a display device for displaying images.

This embodiment can be freely combined with any of the other embodiments.

### Embodiment 4

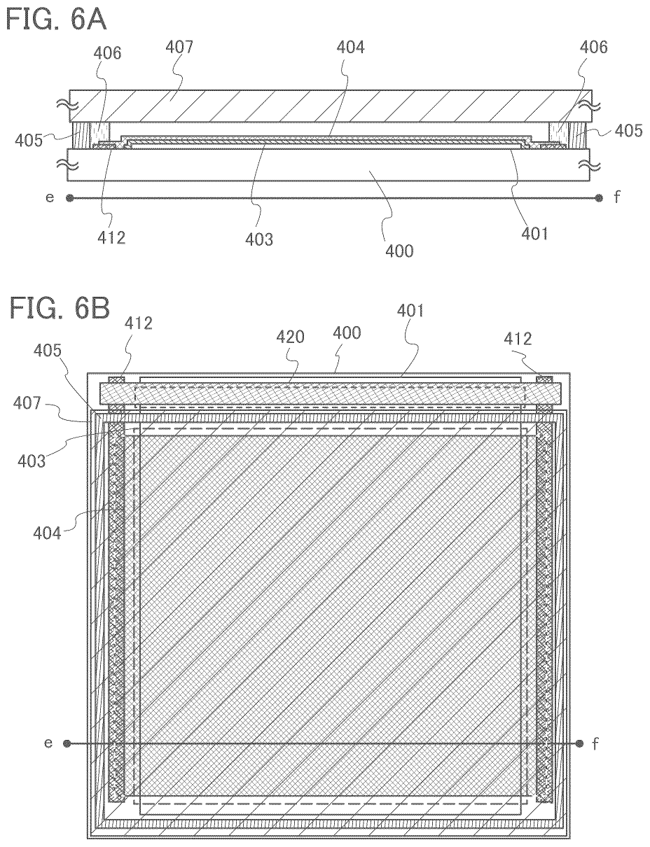

In this embodiment, an example in which the light-emitting device described in Embodiment 2 is used for a lighting device will be described with reference to FIG. 6. FIG. 6B is a top view of the lighting device, and FIG. 6A is a cross-sectional view taken along e-f in FIG. 6B.

In the lighting device in this embodiment, a first electrode 401 is formed over a substrate 400 which is a support and has a light-transmitting property. The first electrode 401 corresponds to the first electrode 101 in Embodiment 2. In the case where light emission is extracted from the first electrode 401 side, the first electrode 401 is formed with a material having a light-transmitting property.

A pad 412 for supplying a voltage to a second electrode 404 is formed over the substrate 400.

An EL layer 403 is formed over the first electrode 401. The EL layer 403 has a structure corresponding to the structure of the EL layer 103 in Embodiment 2, or the structure in which the light-emitting units 511 and 512 are combined with the charge-generation layer 513. Note that for these structures, the corresponding description can be referred to.

The second electrode 404 is formed to cover the EL layer 403. The second electrode 404 corresponds to the second electrode 102 in Embodiment 2. In the case where light-emission is extracted from the first electrode 401 side, the second electrode 404 is formed with a material having high reflectivity. The second electrode 404 is supplied with a voltage when connected to the pad 412.

As described above, the lighting device described in this embodiment includes a light-emitting device including the first electrode 401, the EL layer 403, and the second electrode 404. Since the light-emitting device is alight-emitting device with high emission efficiency, the lighting device in this embodiment can be a lighting device with low power consumption.

The substrate 400 over which the light-emitting device having the above structure is formed is fixed to a sealing substrate 407 with sealants 405 and 406 and sealing is performed, whereby the lighting device is completed. It is possible to use only either the sealant 405 or 406. In addition, the inner sealant 406 (not illustrated in FIG. 6B) can be mixed with a desiccant, which enables moisture to be adsorbed, resulting in improved reliability.

When parts of the pad 412 and the first electrode 401 are provided to extend to the outside of the sealants 405 and 406, those can serve as external input terminals. An IC chip 420 mounted with a converter or the like may be provided over the external input terminals.

The lighting device described in this embodiment uses the light-emitting device described in Embodiment 2 as an EL device; thus, the lighting device can have low power consumption.

### Embodiment 5

In this embodiment, examples of electronic devices each partly including the light-emitting device described in Embodiment 2 are described. The light-emitting device described in Embodiment 2 is a light-emitting device with a high emission efficiency and low power consumption. As a result, the electronic devices described in this embodiment can be electronic devices each including a light-emitting portion with low power consumption.

Examples of electronic devices to which the light-emitting device is applied include television devices (also referred to as TV or television receivers), monitors for computers and the like, digital cameras, digital video cameras, digital photo frames, mobile phones (also referred to as portable telephones or portable telephone devices), portable game machines, portable information terminals, audio playback devices, and large game machines typified by pachinko machines. Specific examples of these electronic devices are shown below.

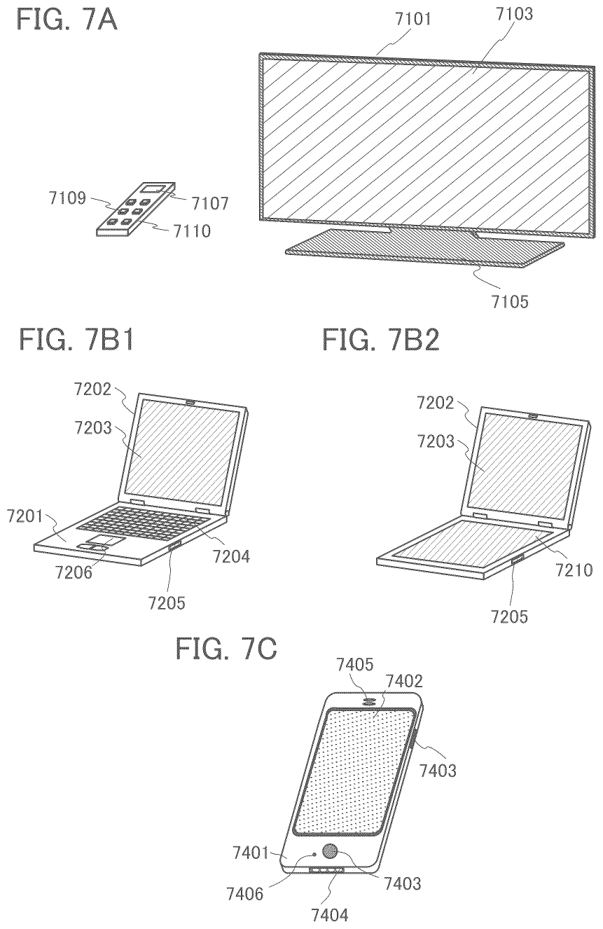

FIG. 7A shows an example of a television device. In the television device, a display portion 7103 is incorporated in a housing 7101. Here, a structure in which the housing 7101 is supported by a stand 7105 is shown. Images can be displayed on the display portion 7103, and the light-emitting devices described in Embodiment 2 are arranged in a matrix in the display portion 7103.

The television device can be operated with an operation switch of the housing 7101 or a separate remote controller 7110. With operation keys 7109 of the remote controller 7110, channels and volume can be operated and images displayed on the display portion 7103 can be operated. Furthermore, a structure may be employed in which the remote controller 7110 is provided with a display portion 7107 for displaying data output from the remote controller 7110.

Note that the television device has a structure of including a receiver, a modem, and the like. With the use of the receiver, a general television broadcast can be received, and moreover, when the television device is connected to a communication network with or without a wire via the modem, one-way (from a sender to a receiver) or two-way (between a sender and a receiver or between receivers) data communication can be performed.

FIG. 7B1 is a computer which includes a main body 7201, a housing 7202, a display portion 7203, a keyboard 7204, an external connection port 7205, a pointing device 7206, and the like. Note that this computer is fabricated using the light-emitting devices described in Embodiment 2 arranged in a matrix in the display portion 7203. The computer in FIG. 7B1 may be such a mode as illustrated in FIG. 7B2. The computer in FIG. 7B2 is provided with a second display portion 7210 instead of the keyboard 7204 and the pointing device 7206. The second display portion 7210 is of a touch-panel type, and input can be performed by operating display for input displayed on the second display portion 7210 with a finger or a dedicated pen. The second display portion 7210 can also display images other than the display for input. The display portion 7203 may also be a touch panel. Connecting the two screens with a hinge can prevent troubles such as a crack in or damage to the screens caused when the computer is stored or carried.

FIG. 7C shows an example of a portable terminal. A mobile phone includes operation buttons 7403, an external connection port 7404, a speaker 7405, a microphone 7406, and the like in addition to a display portion 7402 incorporated in a housing 7401. Note that a mobile phone includes the display portion 7402 which is fabricated by arranging the light-emitting devices described in Embodiment 2 in a matrix.

The portable terminal illustrated in FIG. 7C may have a structure in which information can be input by touching the display portion 7402 with a finger, a touch pen, or the like. In this case, operations such as making a call and creating an e-mail can be performed by touching the display portion 7402 with a finger, a touch pen, or the like.

The display portion 7402 has mainly three screen modes. The first one is a display mode mainly for displaying images, and the second one is an input mode mainly for inputting data typified by text. The third one is a display+input mode in which two modes of the display mode and the input mode are combined.

For example, in the case of making a call or creating an e-mail, a text input mode mainly for inputting text is selected for the display portion 7402 so that an operation of inputting text displayed on the screen may be performed. In this case, it is preferable to display a keyboard or number buttons on almost the entire screen of the display portion 7402.

When a sensing device including a sensor for sensing inclination typified by a gyroscope sensor or an acceleration sensor, is provided inside the portable terminal, screen display of the display portion 7402 can be automatically changed by determining the orientation of the portable terminal (vertically or horizontally).

The screen modes are changed by touching the display portion 7402 or operating the operation buttons 7403 of the housing 7401. Alternatively, the screen modes can be changed depending on the kind of image displayed on the display portion 7402. For example, when a signal of an image displayed on the display portion is moving image data, the screen mode is changed to the display mode, and when the signal is text data, the screen mode is changed to the input mode.

Moreover, in the input mode, when input by the touch operation of the display portion 7402 is not performed for a certain period while a signal sensed by an optical sensor in the display portion 7402 is sensed, the screen mode may be controlled so as to be changed from the input mode to the display mode.

The display portion 7402 can also function as an image sensor. For example, an image of a palm print, a fingerprint, or the like is taken when the display portion 7402 is touched with the palm or the finger, whereby personal authentication can be performed. Furthermore, by using a backlight which emits near-infrared light or a sensing light source which emits near-infrared light in the display portion, an image of a finger vein, a palm vein, or the like can be taken.

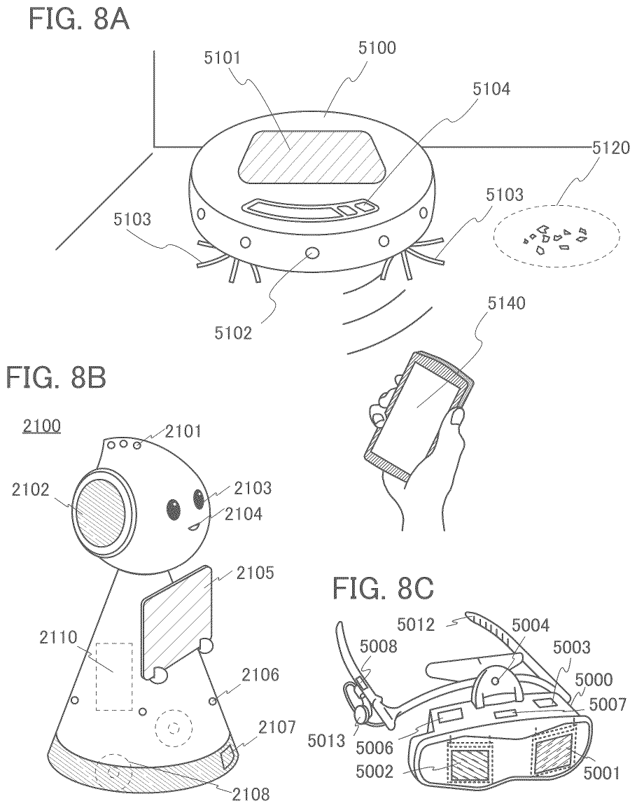

FIG. 8A is a schematic view showing an example of a cleaning robot.

A cleaning robot 5100 includes a display 5101 placed on its top surface, a plurality of cameras 5102 placed on its side surface, a brush 5103, and operation buttons 5104. Although not illustrated, the bottom surface of the cleaning robot 5100 is provided with a tire, an inlet, and the like. Furthermore, the cleaning robot 5100 includes various sensors such as an infrared sensor, an ultrasonic sensor, an acceleration sensor, a piezoelectric sensor, an optical sensor, and a gyroscope sensor. In addition, the cleaning robot 5100 has a wireless communication means.

The cleaning robot 5100 is self-propelled, detects dust 5120, and sucks up the dust through the inlet provided on the bottom surface.

The cleaning robot 5100 can judge whether there is an obstacle such as a wall, furniture, or a step by analyzing images taken by the cameras 5102. When an object that is likely to be caught in the brush 5103, such as a wire, is detected by image analysis, the rotation of the brush 5103 can be stopped.

The display 5101 can display the remaining capacity of a battery, the amount of vacuumed dust, and the like. The display 5101 may display a path on which the cleaning robot 5100 has run. The display 5101 may be a touch panel, and the operation buttons 5104 may be provided on the display 5101.

The cleaning robot 5100 can communicate with a portable electronic device 5140. The portable electronic device 5140 can display images taken by the cameras 5102. Accordingly, an owner of the cleaning robot 5100 can monitor the room even from the outside. The display on the display 5101 can be checked by the portable electronic device 5140.

The light-emitting apparatus of one embodiment of the present invention can be used for the display 5101.

A robot 2100 illustrated in FIG. 8B includes an arithmetic device 2110, an illuminance sensor 2101, a microphone 2102, an upper camera 2103, a speaker 2104, a display 2105, a lower camera 2106, an obstacle sensor 2107, and a moving mechanism 2108.

The microphone 2102 has a function of detecting a speaking voice of a user, an environmental sound, and the like. The speaker 2104 also has a function of outputting sound. The robot 2100 can communicate with a user using the microphone 2102 and the speaker 2104.

The display 2105 has a function of displaying various kinds of information. The robot 2100 can display information desired by a user on the display 2105. The display 2105 may be provided with a touch panel. Moreover, the display 2105 may be a detachable information terminal, in which case charging and data communication can be performed when the display 2105 is set at the home position of the robot 2100.

The upper camera 2103 and the lower camera 2106 each have a function of taking an image of the surroundings of the robot 2100. The obstacle sensor 2107 can detect the presence of an obstacle in the direction where the robot 2100 advances with the moving mechanism 2108. The robot 2100 can move safely by recognizing the surroundings with the upper camera 2103, the lower camera 2106, and the obstacle sensor 2107. The light-emitting apparatus of one embodiment of the present invention can be used for the display 2105.

FIG. 8C shows an example of a goggle-type display. The goggle-type display includes, for example, a housing 5000, a display portion 5001, a speaker 5003, an LED lamp 5004, a connection terminal 5006, a sensor 5007 (a sensor having a function of measuring force, displacement, position, speed, acceleration, angular velocity, rotational frequency, distance, light, liquid, magnetism, temperature, a chemical substance, sound, time, hardness, an electric field, current, voltage, power, radiation, flow rate, humidity, gradient, oscillation, odor, or infrared ray), a microphone 5008, a display portion 5002, a support 5012, and an earphone 5013.

The light-emitting apparatus of one embodiment of the present invention can be used for the display portion 5001 and the display portion 5002.

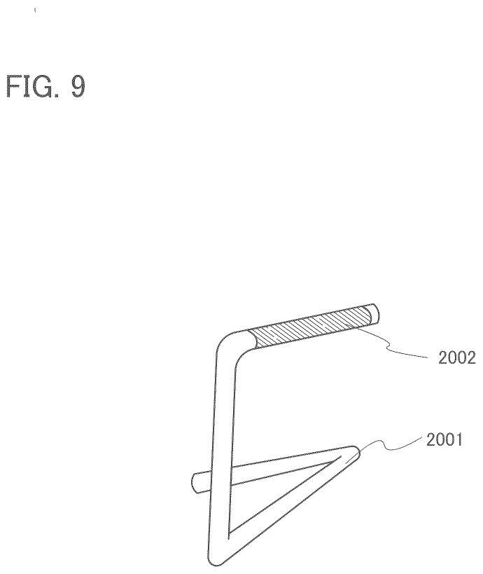

FIG. 9 shows an example in which the light-emitting device described in Embodiment 2 is used for a table lamp which is a lighting device. The table lamp illustrated in FIG. 9 includes a housing 2001 and a light source 2002, and the lighting device described in Embodiment 3 may be used for the light source 2002.

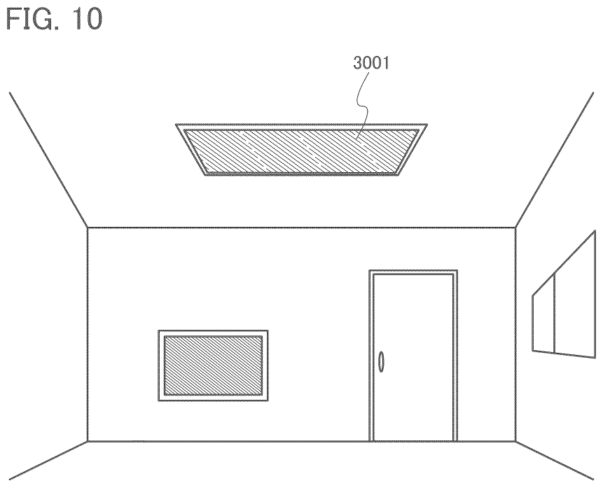

FIG. 10 shows an example in which the light-emitting device described in Embodiment 2 is used for an indoor lighting device 3001. Since the light-emitting device described in Embodiment 2 is a light-emitting device with high emission efficiency, the lighting device can have low power consumption. Furthermore, the light-emitting device described in Embodiment 2 can have a larger area, and thus can be used for a large-area lighting device. Furthermore, the light-emitting device described in Embodiment 2 is thin, and thus can be used for a lighting device having a reduced thickness.

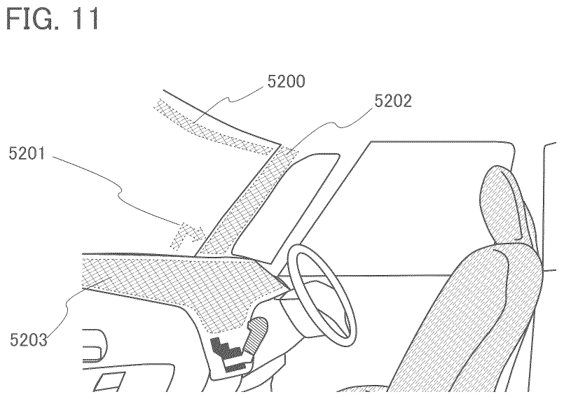

The light-emitting device described in Embodiment 2 can also be incorporated in an automobile windshield or an automobile dashboard. FIG. 11 illustrates one mode in which the light-emitting device described in Embodiment 2 is used for a windshield and a dashboard of an automobile. A display region 5200 to a display region 5203 are each a display region provided using the light-emitting device described in Embodiment 2.

The display region 5200 and the display region 5201 are display devices provided in the automobile windshield, in which the light-emitting devices described in Embodiment 2 are incorporated. When the light-emitting devices described in Embodiment 2 are fabricated using electrodes having light-transmitting properties as a first electrode and a second electrode, what is called see-through display devices, through which the opposite side can be seen, can be obtained. See-through display can be provided without hindering the vision even when being provided in the automobile windshield. Note that in the case where a driving transistor is provided, a transistor having a light-transmitting property typified by an organic transistor using an organic semiconductor material or a transistor using an oxide semiconductor, is preferably used.

The display region 5202 is a display device provided in a pillar portion, in which the light-emitting devices described in Embodiment 2 are incorporated. The display region 5202 can compensate for the view hindered by the pillar by displaying an image taken by an imaging means provided on the car body. Similarly, the display region 5203 provided in the dashboard portion can compensate for the view hindered by the car body by displaying an image taken by an imaging means provided on the outside of the automobile. Thus, blind areas can be compensated for and the safety can be enhanced. Showing an image so as to compensate for the area that cannot be seen makes it possible to confirm safety more naturally and comfortably.

The display region 5203 can provide a variety of kinds of information, such as navigation data, speed, rotational frequency, a mileage, a fuel level, a gearshift state, and air-condition setting. The content, layout, and the like of the display can be changed freely in accordance with the preference of a user. Note that such information can also be provided on the display region 5200 to the display region 5202. The display region 5200 to the display region 5203 can also be used as lighting devices.

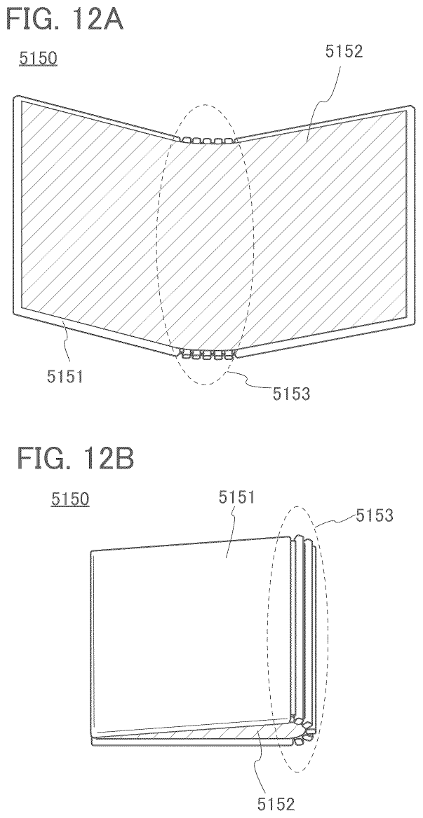

FIG. 12A and FIG. 12B illustrate a foldable portable information terminal 5150. The foldable portable information terminal 5150 includes a housing 5151, a display region 5152, and a bend portion 5153. FIG. 12A illustrates the portable information terminal 5150 that is opened. FIG. 12B illustrates the portable information terminal that is folded. The portable information terminal 5150 is compact in size and has excellent portability when folded, despite its large display region 5152.

The display region 5152 can be folded in half with the bend portion 5153. The bend portion 5153 includes a flexible member and a plurality of supporting members, and when the display region is folded, the flexible member expands and the bend portion 5153 has a radius of curvature of 2 mm or more, preferably 3 mm or more.

Note that the display region 5152 may be a touch panel (an input/output device) including a touch sensor (an input device). The light-emitting apparatus of one embodiment of the present invention can be used for the display region 5152.

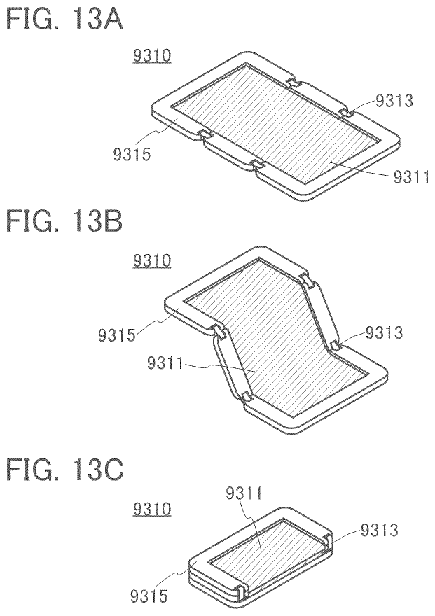

FIG. 13A to FIG. 13C illustrate a foldable portable information terminal 9310. FIG. 13A illustrates the portable information terminal 9310 that is opened. FIG. 13B illustrates the portable information terminal 9310 that is in the state of being changed from one of an opened state and a folded state to the other. FIG. 13C illustrates the portable information terminal 9310 that is folded. The portable information terminal 9310 is excellent in portability when folded, and is excellent in display browsability when opened because of a seamless large display region.

A display panel 9311 is supported by three housings 9315 joined together by hinges 9313. Note that the display panel 9311 may be a touch panel (an input/output device) including a touch sensor (an input device). By folding the display panel 9311 at the hinges 9313 between two housings 9315, the portable information terminal 9310 can be reversibly changed in shape from the opened state to the folded state. A light-emitting apparatus of one embodiment of the present invention can be used for the display panel 9311.

Note that the structures described in this embodiment can be combined with the structures described in any of Embodiment 1 to Embodiment 4 as appropriate.

The organic compound of one embodiment of the present invention can be used for a photoelectric element such as an organic thin film solar cell (OPV) or an organic photo diode (OPD). Specifically, the organic compound can be used in a carrier-transport layer or a carrier-injection layer because the organic compound has a carrier-transport property. In addition, a mixed film of the organic compound and a donor substance can be used for a charge generation layer. The organic compound is photoexcited and thus can be used for a power generation layer or an active layer.

As described above, the application range of the light-emitting apparatus including the light-emitting device described in Embodiment 2 is so wide that this light-emitting apparatus can be applied to electronic devices in a variety of fields. With the use of the light-emitting device described in Embodiment 2, an electronic device with low power consumption can be obtained.

### Example 1

### Synthesis Example 1

In this synthesis example, a method for synthesizing 3-[4-(dibenzothiophen-4-yl)phenyl]imidazo[1,2-f]phenanthridine (abbreviation: DBTPIPt-II) shown as Structural Formula (100) in Embodiment 1 is specifically described. The structural formula of DBTPIPt-II is shown below.

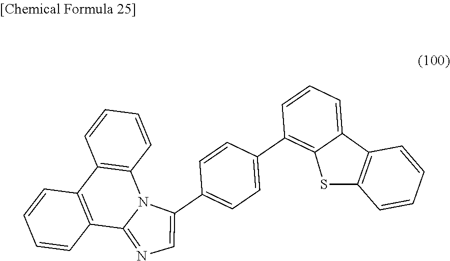 [SMILES: c1ccc2c(c1)sc1c(-c3ccc(-c4cnc5c6ccccc6c6ccccc6n45)cc3)cccc12]

### Step 1; Synthesis of 3-[4-(dibenzothiophen-4-yl)phenyl]imidazo[1,2-J]phenanthridine (abbreviation: DBTPIPt-II)

Into a 200 mL three-neck flask were put 0.80 g (2.7 mmol) of 3-bromoimidazo[1,2-f]phenanthridine, 1.2 g (4.0 mmol) of 4-(dibenzothiophen-4-yl)phenylboronic acid, 1.1 g (7.6 mmol) of potassium carbonate, 27 mL of toluene, 3 mL of ethanol, and 3 mL of water. The mixture was degassed by being stirred under reduced pressure, and the air in the flask was replaced with nitrogen. To the mixture, 0.12 g (0.10 mmol) of tetrakis(triphenylphosphine)palladium(0) was added, and the mixture was stirred under a nitrogen stream at 80° C. for 6 hours and then refluxed at 100° C. for 11 hours. After the refluxing, water was added to the mixture to separate an aqueous layer and an organic layer, and the aqueous layer was subjected to extraction with toluene. The obtained extract and organic layer were combined and washed with a saturated aqueous solution of sodium hydrogen carbonate and saturated saline, and then the organic layer was dried with magnesium sulfate. The mixture was gravity-filtered and the filtrate was concentrated to give an oily substance. The obtained oily substance was purified by alumina column chromatography (toluene:ethyl acetate=50:1) to give an oily substance. Methanol was added to the oily substance, the oily substance to which methanol was added was irradiated with ultrasonic waves, and the precipitated solid was collected to give 1.0 g of a target white powder at a yield of 79%. Purification by a train sublimation method was performed by heating 1.0 g of the obtained white powder at 260° C. under a pressure of 3.2 Pa with an argon flow rate of 5.0 mL/min for 14 hours. After the sublimation purification, 0.89 g of a white solid was obtained at a collection rate of 87%. The synthesis scheme of Step 1 is shown below.

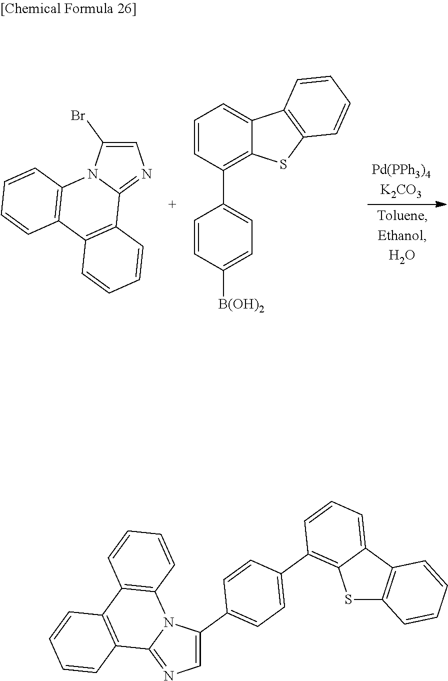 [SMILES: Brc1cnc2c3ccccc3c3ccccc3n12 ; OB(O)c1ccc(-c2cccc3c2sc2ccccc23)cc1 ; c1ccc2c(c1)sc1c(-c3ccc(-c4cnc5c6ccccc6c6ccccc6n45)cc3)cccc12]

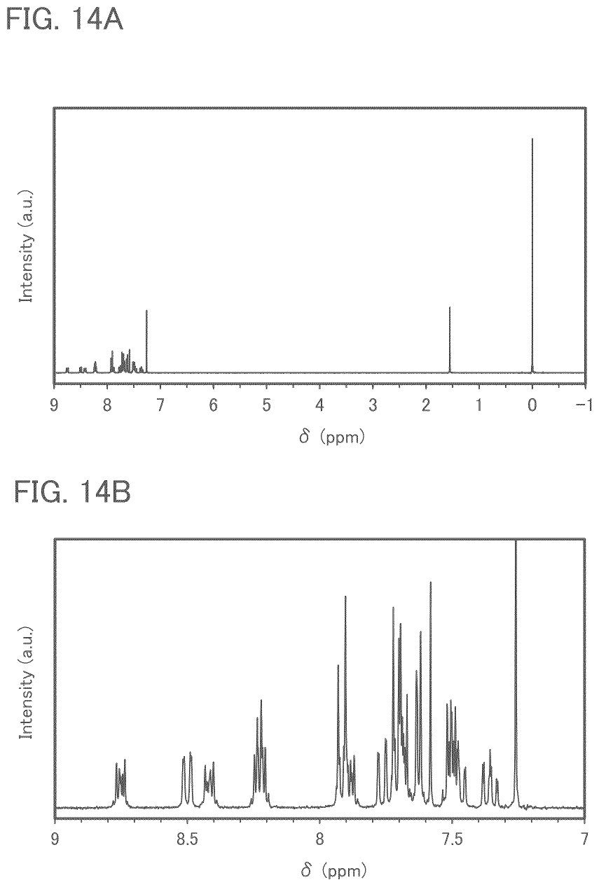

Analysis results by nuclear magnetic resonance spectroscopy (¹H-NMR) of the white solid obtained in Step 1 are shown below. FIG. 14A and FIG. 14B show ¹H-NMR charts. In this synthesis example, the above indicates that DBTPIPt-II which is the organic compound of the present invention was obtained.

¹H NMR (CDCl₃, 300 MHz): δ=7.33-7.38 (m, 1H), 7.45-7.54 (m, 3H), 7.58 (s, 1H), 7.61-7.78 (m, 7H), 7.86-7.93 (m, 3H), 8.19-8.26 (m, 2H), 8.39-8.43 (m, 1H), 8.50 (dd, J=8.4 Hz, 1.5 Hz, 1H), 8.74-8.78 (m, 1H).

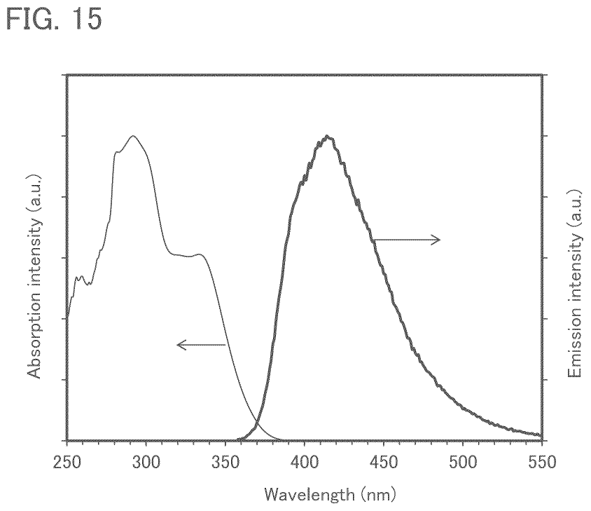

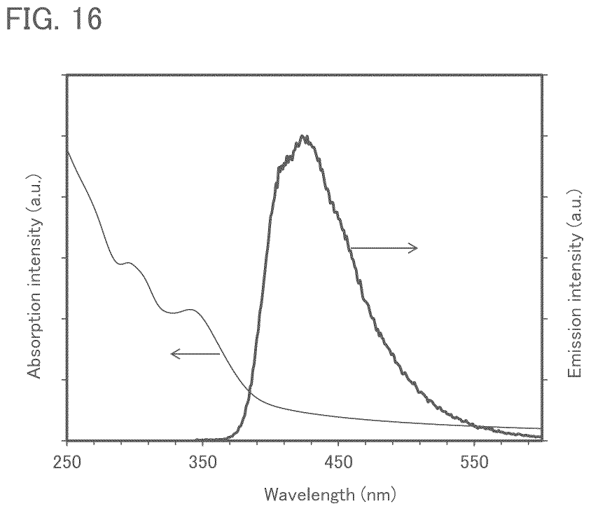

Next, FIG. 15 shows an absorption spectrum and an emission spectrum of DBTPIPt-II in a toluene solution. FIG. 16 shows an absorption spectrum and an emission spectrum of a thin film. The solid thin film was formed over a quartz substrate by a vacuum evaporation method. The absorption spectrum was measured with an ultraviolet-visible spectrophotometer (V550, manufactured by JASCO Corporation). The absorption spectrum in a solution state was obtained by subtraction of a measured absorption spectrum of a solvent alone in a quartz cell from a measured absorption spectrum of the solution of DBTPIPt-II in a quartz cell. The absorption spectrum of the thin film was obtained by subtraction of an absorption spectrum of a quartz substrate from an absorption spectrum of DBTPIPt-II deposited over a quartz substrate. The emission spectrum was measured with a fluorescence spectrophotometer (FS920, manufactured by Hamamatsu Photonics K.K.).

FIG. 15 shows that the toluene solution of DBTPIPt-II has absorption peaks at around 333 nm and 292 nm and an emission wavelength peak at 415 nm (excitation wavelength: 338 nm). Furthermore, FIG. 16 shows that the thin film of DBTPIPt-II has absorption peaks at around 340 nm, 295 nm, and 242 nm and emission wavelength peaks at around 412 nm and 424 nm (excitation wavelength: 340 nm). The result revealed that DBTPIPt-II which is the organic compound of one embodiment of the present invention can be effectively used also as a host-transport material of each of a light-emitting substance and a substance that emits fluorescence in the visible region.

Furthermore, the thin film of DBTPIPt-II was found to have a good film quality with little change, hardly being aggregated even under air.

Next, the HOMO level and the LUMO level of DBTPIPt-II were calculated on the basis of cyclic voltammetry (CV) measurement. The calculation method is shown below.

An electrochemical analyzer (model number: ALS model 600A or 600C, manufactured by BAS Inc.) was used as a measurement apparatus. To prepare a solution used in the CV measurement, dehydrated dimethylformamide (DMF) (manufactured by Sigma-Aldrich Inc., 99.8%, catalog No. 22705-6) was used as a solvent, tetra-n-butylammonium perchlorate (n-Bu₄NClO₄) (manufactured by Tokyo Chemical Industry Co., Ltd., catalog No. T0836) as a supporting electrolyte was dissolved at a concentration of 100 mmol/L, and the object to be measured was dissolved at a concentration of 2 mmol/L. A platinum electrode (PTE platinum electrode, manufactured by BAS Inc.) was used as a working electrode, another platinum electrode (Pt counter electrode for VC-3 (5 cm), manufactured by BAS Inc.) was used as an auxiliary electrode, and an Ag/Ag⁺ electrode (RE7 reference electrode for non-aqueous solvent, manufactured by BAS Inc.) was used as a reference electrode. Note that the measurement was performed at room temperature (20 to 25° C.). The scan speed in the CV measurement was fixed to 0.1 V/sec, and an oxidation potential Ea [V] and a reduction potential Ec [V] with respect to the reference electrode were measured. Ea is an intermediate potential of an oxidation-reduction wave, and Ec is an intermediate potential of a reduction-oxidation wave. Here, since the potential energy of the reference electrode used in this example with respect to the vacuum level is known to be −4.94 [eV], the HOMO level and the LUMO level can be calculated by the following formulae: HOMO level [eV]=−4.94−Ea and LUMO level [eV]=−4.94−Ec.

Furthermore, CV measurement was repeated 100 times, and the oxidation-reduction wave in the hundredth cycle was compared with the oxidation-reduction wave in the first cycle to examine the electrical stability of the compound.

The measurement result of the oxidation potential Ea [V] shows that the HOMO level of DBTPIPt-II is −5.89 eV. Furthermore, the measurement result of the reduction potential Ec [V] shows that the LUMO level of DBTPIPt-II is −2.35 eV. In addition, when the oxidation-reduction wave was repeatedly measured, 89% of the peak intensity was maintained in the Ea measurement; whereby DBTPIPt-II was confirmed to have extremely high resistance to oxidation.

### Example 2

### Synthesis Example 2

In this synthesis example, a method for synthesizing 3-[4-(carbazol-9-yl)phenyl]imidazo[1,2-f]phenanthridine (abbreviation: CzPIPt) shown as Structural Formula (135) in Embodiment 1 is specifically described. The structural formula of CzPIPt is shown below.

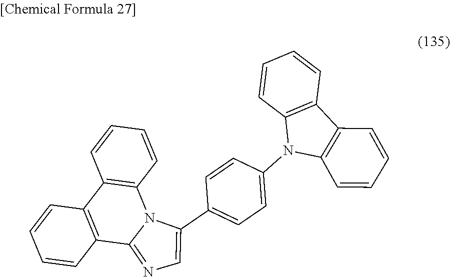 [SMILES: c1ccc2c(c1)c1ccccc1n1c(-c3ccc(-n4c5ccccc5c5ccccc54)cc3)cnc21]

### Step 1; Synthesis of 3-[4-(carbazol-9-yl)phenyl]imidazo[1,2-f]phenanthridine (abbreviation: CzPIPt)

Into a 200 mL three-neck flask were put 0.80 g (2.7 mmol) of 3-bromoimidazo[1,2-f]phenanthridine, 1.3 g (4.4 mmol) of 4-(carbazol-9-yl)phenylboronic acid, 1.0 g (7.5 mmol) of potassium carbonate, 30 mL of toluene, 3 mL of ethanol, and 3 mL of water. The mixture was degassed by being stirred under reduced pressure, and the air in the flask was replaced with nitrogen. To the mixture, 0.17 g (0.15 mmol) of tetrakis(triphenylphosphine)palladium(0) was added, and the mixture was stirred under a nitrogen stream at 80° C. for 9 hours and then refluxed at 100° C. for 6 hours. After the refluxing, water was added to the mixture to separate an aqueous layer and an organic layer, and the aqueous layer was subjected to extraction with toluene. The obtained extract and organic layer were combined and washed with a saturated aqueous solution of sodium hydrogen carbonate and saturated saline, and then dried with magnesium sulfate. This mixture was gravity-filtered and the filtrate was concentrated to give a solid. The obtained solid was purified by silica gel column chromatography (toluene:ethyl acetate=20:1) and alumina column chromatography (toluene:ethyl acetate=50:1) to give a solid. The solid was recrystallized with toluene to give 0.86 g of a target white powder in a yield of 69%. By a train sublimation method, 0.85 g of the obtained white powder was purified by sublimation at 260° C. under a pressure of 3.0 Pa with an argon flow rate of 5.0 mE/min for 16 hours. After the sublimation purification, 0.77 g of a white solid was obtained at a collection rate of 91%. The synthesis scheme of Step 1 is shown below.

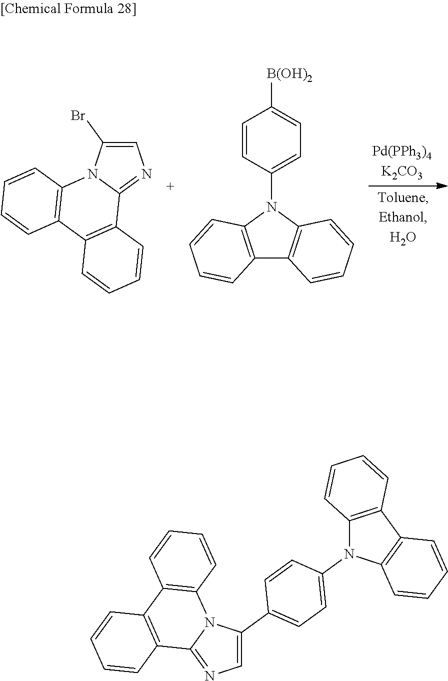 [SMILES: Brc1cnc2c3ccccc3c3ccccc3n12 ; OB(O)c1ccc(-n2c3ccccc3c3ccccc32)cc1 ; c1ccc2c(c1)c1ccccc1n1c(-c3ccc(-n4c5ccccc5c5ccccc54)cc3)cnc21]

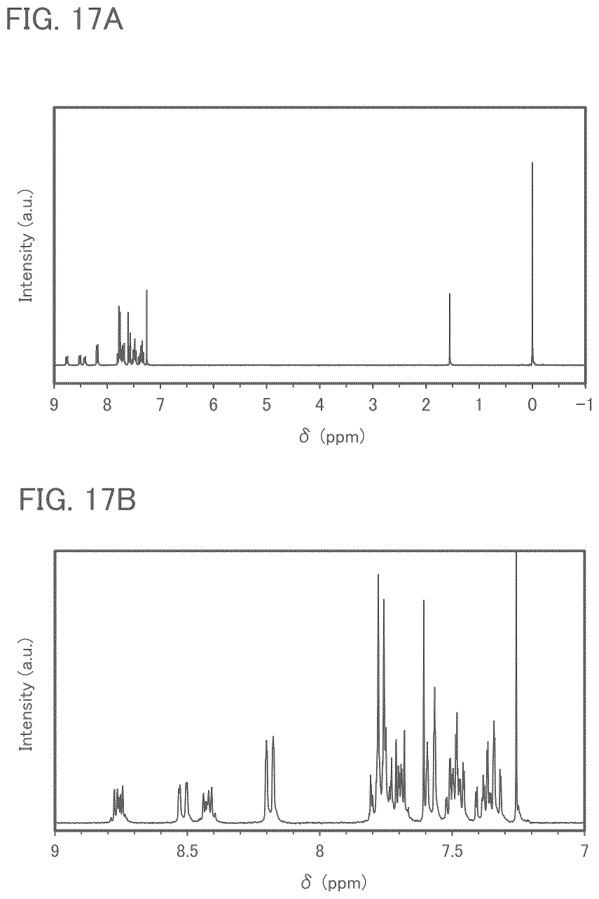

Analysis results by nuclear magnetic resonance spectroscopy (¹H-NMR) of the white solid obtained in Step 1 are shown below. FIG. 17A and FIG. 17B show ¹H-NMR charts. In this synthesis example, the above indicates that CzPIPt which is the organic compound of the present invention was obtained.

¹H NMR (CDCl₃, 300 MHz): δ=7.32-7.41 (m, 3H), 7.45-7.52 (m, 3H), 7.57-7.61 (m, 3H), 7.67-7.81 (m, 7H), 8.19 (d, J=7.8 Hz, 2H), 8.40-8.44 (m, 1H), 8.52 (dd, J=8.4 Hz, 1.5 Hz, 1H), 8.74-8.79 (m, 1H).

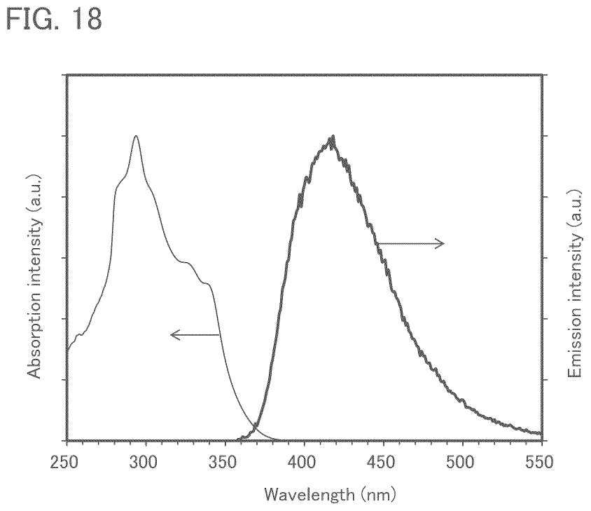

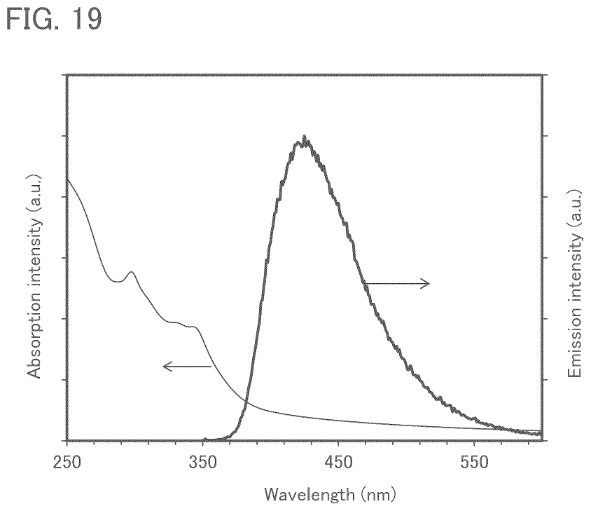

Next, FIG. 18 shows an absorption spectrum and an emission spectrum of CzPIPt in a toluene solution. FIG. 19 shows an absorption spectrum and an emission spectrum of a thin film. The solid thin film was formed over a quartz substrate by a vacuum evaporation method. The absorption spectrum was measured with an ultraviolet-visible spectrophotometer (V550, manufactured by JASCO Corporation). The absorption spectrum in a solution state was obtained by subtraction of a measured absorption spectrum of a solvent alone in a quartz cell from a measured absorption spectrum of the solution of CzPIPt in a quartz cell. The absorption spectrum of the thin film was obtained by subtraction of an absorption spectrum of a quartz substrate from an absorption spectrum of CzPIPt deposited over a quartz substrate. The emission spectrum was measured using a fluorescence spectrophotometer (FS920, manufactured by Hamamatsu Photonics K.K.).

FIG. 18 shows that the toluene solution of CzPIPt has absorption peaks at around 340 nm, 327 nm, and 294 nm and an emission wavelength peak at 416 nm (excitation wavelength: 342 nm). Furthermore, FIG. 19 shows that the thin film of CzPIPt has absorption peaks at around 344 nm, 331 nm, and 297 nm and emission wavelength peaks at around 425 nm (excitation wavelength: 343 nm). The result revealed that CzPIPt which is the organic compound of one embodiment of the present invention can be effectively used as a host-transport material of each of a light-emitting substance and a substance that emits fluorescence in the visible region.

Furthermore, the thin film of CzPIPt was found to have a good film quality with little change, hardly being aggregated even under air.

Next, the HOMO level and the LUMO level of CzPIPt were calculated based on a cyclic voltammetry (CV) measurement. Since the calculation method is described in Synthesis Example 1, repeated description is omitted.

As a result, the HOMO level of CzPIPt was found to be −5.89 eV by the measurement of the oxidation potential Ea [V], and the LUMO level of CzPIPt was found to be −2.30 eV by the measurement of the reduction potential Ec [V].

### Example 3

### Synthesis Example 3

In this synthesis example, a method for synthesizing 3-(1,2,4-triazolo[4,3-f]phenanthridin-3-yl)triphenylamine (abbreviation: mDPhATPt) shown as Structural Formula (165) in Embodiment 1 is specifically described. The structural formula of mDPhATPt is shown below.

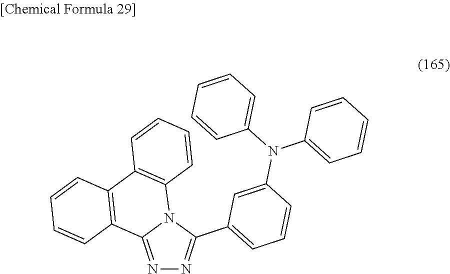 [SMILES: c1ccc(N(c2ccccc2)c2cccc(-c3nnc4c5ccccc5c5ccccc5n34)c2)cc1]

### Step 1; Synthesis of 3-(1,2,4-triazolo[4,3-f]phenanthridin-3-yl)triphenylamine (abbreviation: mDPhATPt)

Into a 100 mL three-neck flask were put 1.5 g (3.9 mmol) of 3-(3-bromophenyl)-1,2,4-triazolo[4,3-f]phenanthridine, 0.67 g (4.0 mmol) of diphenylamine, 0.14 g (0.74 mmol) of copper(I) iodide, 0.14 g (0.52 mmol) of 18-crown-6-ether, 1.1 g (8.2 mmol) of potassium carbonate, and 3 mL of 1,3-dimethyl-3,4,5,6-tetrahydro-2(1H)-pyrimidinone (abbreviation: DMPU). The mixture was stirred under a nitrogen stream at 180° C. for 17 hours. After the stirring, the mixture was cooled to room temperature, and chloroform was added. The mixture was washed with water, a saturated aqueous solution of sodium hydrogen carbonate, and saturated saline, and the organic layer was dried with magnesium sulfate. The mixture was subjected to gravity-filtration and the filtrate was concentrated to give an oily substance. The obtained oily substance was purified by silica gel column chromatography (toluene:ethyl acetate=10:1). Methanol was added to the solid, the solid to which methanol was added was irradiated with ultrasonic waves, and a solid was collected. The obtained solid was recrystallized with toluene to give 0.79 g of a target pale yellow powder at a yield of 43%.

Purification by a train sublimation method was performed by heating 0.77 g of the obtained pale yellow powder at 240° C. under a pressure of 3.2 Pa with an argon gas flow rate of 5.0 mL/min for 16 hours. After the sublimation purification, 0.63 g of a pale yellow powder was obtained at a collection rate of 82%. The synthesis scheme of Step 1 is shown below.

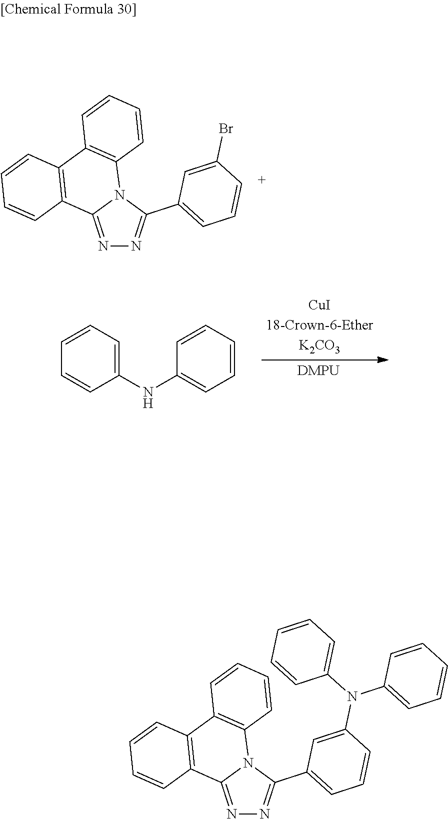 [SMILES: Brc1cccc(-c2nnc3c4ccccc4c4ccccc4n23)c1 ; c1ccc(Nc2ccccc2)cc1 ; c1ccc(N(c2ccccc2)c2cccc(-c3nnc4c5ccccc5c5ccccc5n34)c2)cc1]

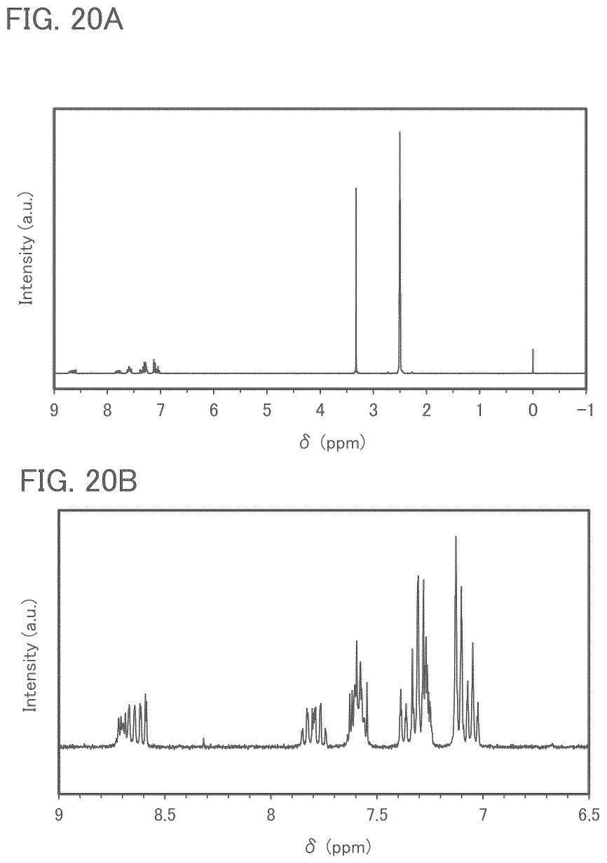

Analysis results by nuclear magnetic resonance spectroscopy (¹H-NMR) of the pale yellow powder obtained in Step 1 above are shown below. FIG. 20A and FIG. 20B show ¹H-NMR charts. In this synthesis example, the above indicates that mDPhATPt which is the organic compound of the present invention was obtained.

¹H NMR (DMSO-d6, 300 MHz): δ=7.02-7.13 (m, 6H), 7.25-7.39 (m, 7H), 7.55-7.64 (m, 4H), 7.74-7.85 (m, 2H), 8.59-8.72 (m, 3H).

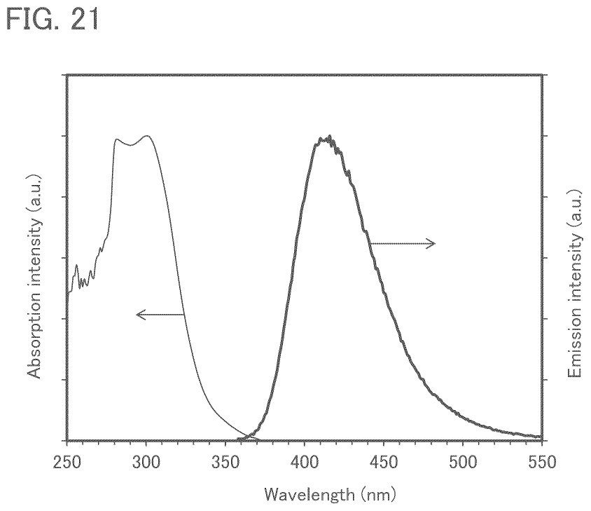

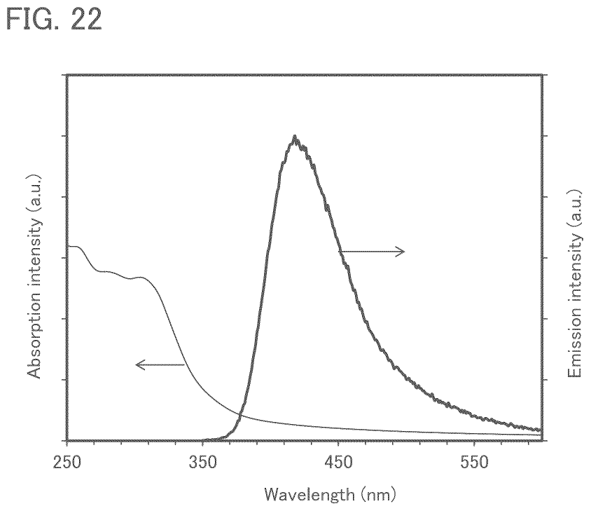

Next, FIG. 21 shows an absorption spectrum and an emission spectrum of mDPhATPt in a toluene solution. FIG. 22 shows an absorption spectrum and an emission spectrum of a thin film. The solid thin film was formed over a quartz substrate by a vacuum evaporation method. The absorption spectrum was measured with an ultraviolet-visible spectrophotometer (V550, manufactured by JASCO Corporation). The absorption spectrum in a solution state was obtained by subtraction of a measured absorption spectrum of a solvent alone in a quartz cell from a measured absorption spectrum of the solution of mDPhATPt in a quartz cell. The absorption spectrum of the thin film was obtained by subtraction of an absorption spectrum of a quartz substrate from an absorption spectrum of mDPhATPt deposited over a quartz substrate. The emission spectrum was measured using a fluorescence spectrophotometer (FS920, manufactured by Hamamatsu Photonics K.K.).

FIG. 21 shows that the toluene solution of mDPhATPt has absorption peaks at around 301 nm and 282 nm and an emission wavelength peak at around 415 nm (excitation wavelength: 306 nm). Furthermore, FIG. 22 shows that the thin film of mDPhATPt has absorption peaks at around 304 nm, 281 nm, and 258 nm and an emission wavelength peak at around 418 nm (excitation wavelength: 307 nm). The result revealed that mDPhATPt which is the organic compound of one embodiment of the present invention can be effectively used also as a host-transport material of each of a light-emitting substance and a substance that emits fluorescence in the visible region.

Furthermore, the thin film of mDPhATPt was found to have a good film quality with little change, hardly being aggregated even under air.

The HOMO level and the LUMO level of mDPhATPt were calculated on the basis of cyclic voltammetry (CV) measurement. Since the calculation method is described in Synthesis Example 1, repeated description is omitted.

The measurement result of the oxidation potential Ea [V] shows that the HOMO level of mDPhATPt is −5.62 eV. Furthermore, the measurement result of the reduction potential Ec [V] shows that the LUMO level of mDPhATPt is −2.36 eV. In addition, when the oxidation-reduction wave was repeatedly measured, 91% of the peak intensity was maintained in the Ea measurement; whereby mDPhATPt was confirmed to have extremely high resistance to oxidation.

### Example 4

In this example, a light-emitting device 1 of one embodiment of the present invention and a comparative light-emitting device 1 that is a comparative example are described. Structural formulae of organic compounds used in this example are shown below.

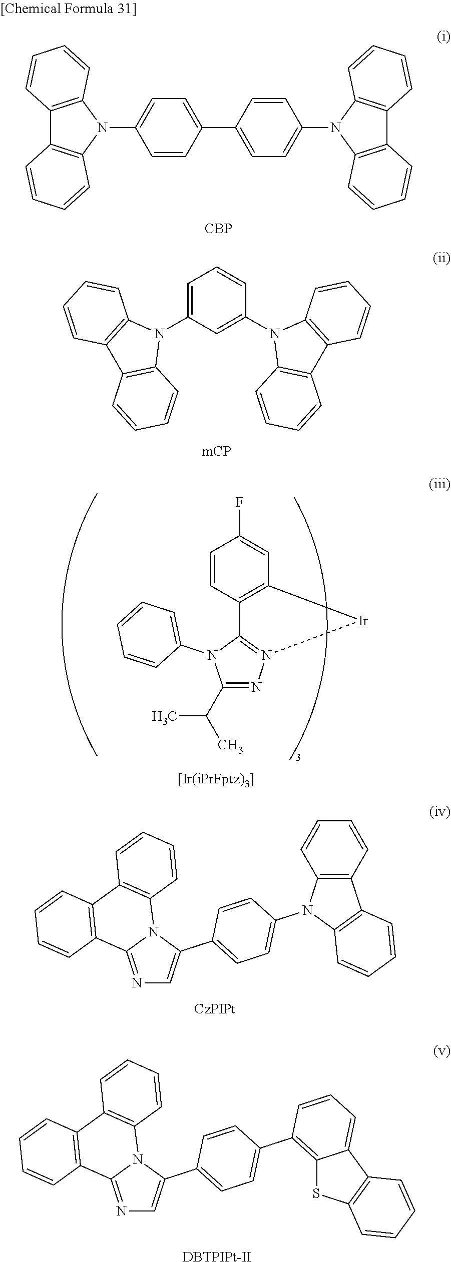

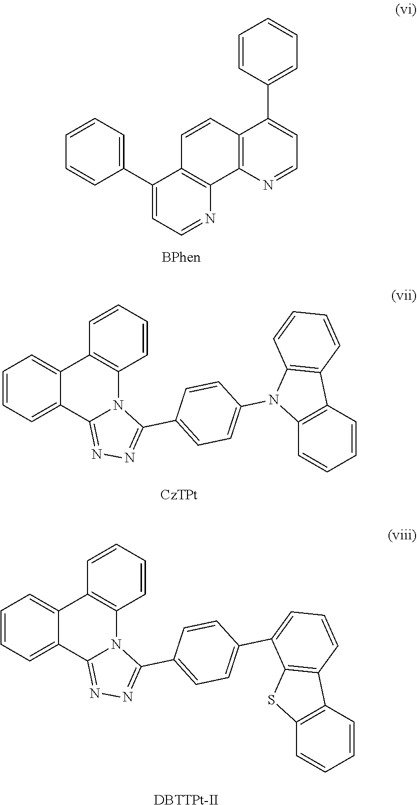 [SMILES: c1ccc(-c2ccnc3c2ccc2c(-c4ccccc4)ccnc23)cc1 ; c1ccc2c(c1)c1ccccc1n1c(-c3ccc(-n4c5ccccc5c5ccccc54)cc3)nnc21 ; c1ccc2c(c1)sc1c(-c3ccc(-c4nnc5c6ccccc6c6ccccc6n45)cc3)cccc12 ; "DBTTPt-II"]

(Method for Fabricating Light-Emitting Device 1)

First, indium tin oxide containing silicon oxide (ITSO) was deposited over a glass substrate by a sputtering method, so that the first electrode 101 was formed. Note that the film thickness was 110 nm and the area of the electrode was 2 mm×2 mm.

Next, in pretreatment for forming the light-emitting device over the substrate, a surface of the substrate was washed with water and baked at 200° C. for 1 hour, and then UV ozone treatment was performed for 370 seconds.

After that, the substrate was transferred into a vacuum evaporation apparatus in which the pressure was reduced to about 1×10⁻⁴ Pa, vacuum baking at 170° C. for 30 minutes was performed in a heating chamber in the vacuum evaporation apparatus, and then the substrate was naturally cooled down for about 30 minutes.

Next, the substrate over which the first electrode 101 was formed was fixed to a substrate holder provided in the vacuum evaporation apparatus so that the surface over which the first electrode 101 was formed faced downward, and 4,4′-di(carbazol-9-yl)biphenyl (abbreviation: CBP) represented by Structural Formula (i) above and molybdenum oxide were co-evaporated over the first electrode 101 to have a weight ratio of 4:2 (=CBP: molybdenum oxide) to a thickness of 60 nm by an evaporation method using resistance heating, whereby the hole-injection layer 111 was formed.

Then, 1,3-bis(carbazol-9-yl)benzene (abbreviation: mCP) represented by Structural Formula (ii) above was deposited by evaporation to a thickness of 20 nm over the hole-injection layer 111 to form the hole-transport layer 112.

Next, mCP and tris[3-(4-fluorophenyl)-5-isopropyl-4-phenyl-4H-1,2,4-triazolato]iridium(III) (abbreviation: [Ir(iPrFptz)₃]) represented by Structural Formula (iii) above were co-evaporated to have a weight ratio of 1:0.08 (=mCP: [Ir(iPrFptz)₃]) to a thickness of 10 nm, whereby a first light-emitting layer was formed. After that, 3-[4-(carbazol-9-yl)phenyl]imidazo[1,2-f]phenanthridine (abbreviation: CzPIPt) represented by Structural Formula (iv) above and [Ir(iPrFptz)₃] were co-evaporated to have a weight ratio of 1:0.08 (=CzPIPt: [Ir(iPrFptz)₃]) to a thickness of 20 nm, whereby a second light-emitting layer was formed. Furthermore, 3-[4-(dibenzothiophen-4-yl)phenyl]imidazo[1,2-f]phenanthridine (abbreviation: DBTPIPt-II) represented by Structural Formula (v) above and [Ir(iPrFptz)₃] were co-evaporated to have a weight ratio of 1:0.08 (=DBTPIPt-II: [Ir(iPrFptz)₃]) to a thickness of 10 nm, whereby a third light-emitting layer was formed. Accordingly, the light-emitting layer 113 was formed.

After that, over the light-emitting layer 113, bathophenanthroline (abbreviation: Bphen) represented by Structural Formula (vi) above was deposited by evaporation to a thickness of 15 nm, whereby the electron-transport layer 114 was formed.

After the formation of the electron-transport layer 114, lithium fluoride (LiF) was deposited by evaporation to a thickness of 1 nm to form the electron-injection layer 115, and then aluminum was deposited by evaporation to a thickness of 200 nm to form the second electrode 102, whereby the light-emitting device 1 of this example was fabricated.

(Method for fabricating light-emitting device 2) A light-emitting device 2 was fabricated in a manner similar to that of the light-emitting device 1 except that DBTPIPt-II was used instead of CzPIPt in the light-emitting device 1.

(Method for Fabricating Comparative Light-Emitting Device 1)

The comparative light-emitting device 1 was fabricated in a manner similar to that of the light-emitting device 1 except that 3-[4-(9H-carbazol-9-yl)phenyl]-1,2,4-triazolo[4,3-f]phenanthridine (abbreviation: CzTPt) represented by Structural Formula (vii) above was used instead of CzPIPt in the light-emitting device 1, and 3-[4-(dibenzothiophen-4-yl)phenyl]-1,2,4-triazolo[4,3-f]phenanthridine (abbreviation: DBTTPt-II) represented by Structural Formula (viii) above was used instead of DBTPIPt-II in the light-emitting device 1.

(Method for Fabricating Comparative Light-Emitting Device 2)

A comparative light-emitting device 2 was fabricated in a manner similar to that of the light-emitting device 2 except that DBTTPt-II was used instead of DBTPIPt-II in the light-emitting device 2.

The stacked-layer structures of the light-emitting device 1, the light-emitting device 2, the comparative light-emitting device 1, and the comparative light-emitting device 2 are listed in the following table.

TABLE 1

| | | | | Film |
|---|---|---|---|---|
| | | Constituent | Weight | thickness |
| Functional layer name | | material | ratio | (nm) |
| Electron-injection layer | | LiF | | 1 |
| Electron-transport layer | | BPhen | | 15 |
| Light-emitting layer | 3 | *2:[Ir(iPrFptz)₃] | 1:0.08 | 10 |
| | 2 | *1:[Ir(iPrFptz)₃] | 1:0.08 | 20 |
| | 1 | mCP:[Ir(iPrFptz)₃] | 1:0.08 | 10 |

| | | | |
|---|---|---|---|
| Hole-transport layer | mCP | | 20 |
| Hole-injection layer | CBP:MoOx | 4:2 | 60 |

Light-emitting device 1 *1: CzPIPt, *2: DBTPIPt-II
Light-emitting device 2 *1: DBTPIPt-II, *2: DBTPIPt-II
Comparative light-emitting device 1 *1: CzTPt, *2: DBTTPt-II
Comparative light-emitting device 2 *1: DBTTPt-II, *2: DBTTPt-II

These light-emitting devices were subjected to sealing with a glass substrate (a sealant was applied to surround the elements, followed by UV treatment and one-hour heat treatment at 80° C. at the time of sealing) in a glove box containing a nitrogen atmosphere so that the light-emitting devices were not exposed to the air. Then, the initial characteristics were measured.

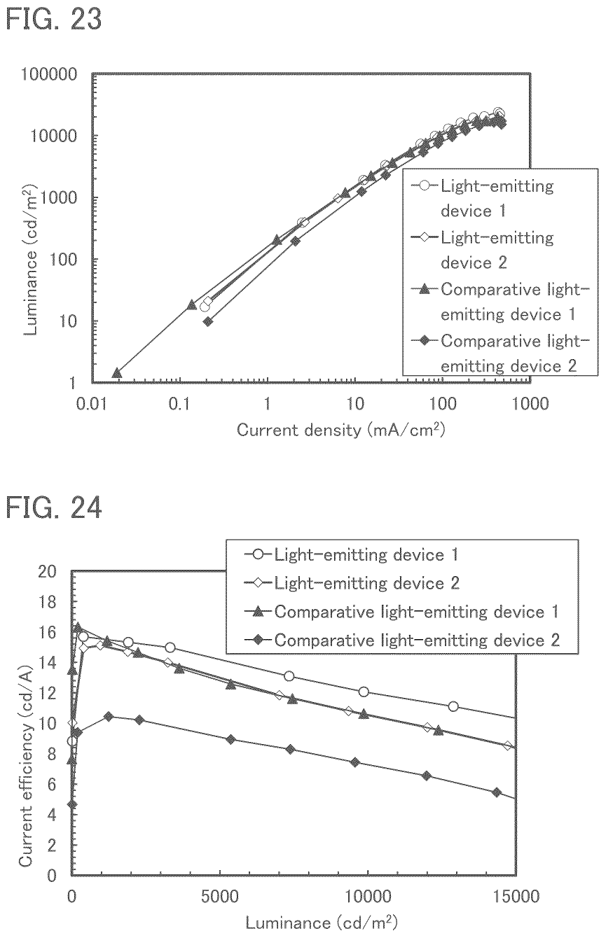

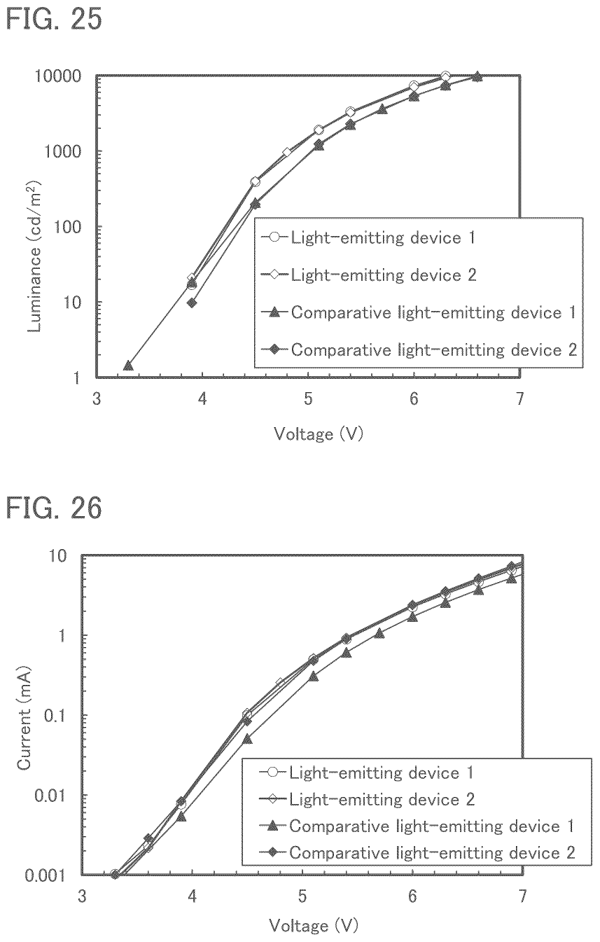

FIG. 23 shows the luminance-current density characteristics of the light-emitting device 1, the light-emitting device 2, the comparative light-emitting device 1, and the comparative light-emitting device 2; FIG. 24 shows the current efficiency-luminance characteristics thereof, FIG. 25 shows the luminance-voltage characteristics thereof, FIG. 26 shows the current-voltage characteristics thereof, FIG. 27 shows the external quantum efficiency-luminance characteristics thereof, and FIG. 28 shows the emission spectra thereof. Table 2 shows main characteristics of the light-emitting device 1 at approximately 1000 cd/m². Luminance and CIE chromaticity were measured with a luminance colorimeter (BM-5A manufactured by TOPCON TECHNOHOUSE CORPORATION), and electroluminescence spectra were measured with a multi-channel spectrometer (PMA-11 manufactured by Hamamatsu Photonics K.K.). Note that the measurements of the light-emitting devices were performed at room temperature (in an atmosphere maintained at 23° C.).

TABLE 2

| | | | | | | | External |
|---|---|---|---|---|---|---|---|
| | | | Current | | | Current | quantum |
| | Voltage | Current | density | Chromaticity | Chromaticity | efficiency | efficiency |
| | (V) | (mA) | (mA/cm²) | x | y | (cd/A) | (%) |
| Light-emitting device 1 | 4.5 | 0.10 | 2.5 | 0.179 | 0.242 | 15.7 | 9.2 |
| Light-emitting device 2 | 4.8 | 0.26 | 6.4 | 0.180 | 0.244 | 15.1 | 8.8 |
| Comparative | 5.1 | 0.31 | 7.7 | 0.185 | 0.257 | 15.4 | 8.4 |
| light-emitting device 1 | | | | | | | |
| Comparative | 5.1 | 0.47 | 11.9 | 0.183 | 0.247 | 10.4 | 5.9 |
| light-emitting device 2 | | | | | | | |

FIG. 28 shows that from the light-emitting device 1 and the light-emitting device 2 of one embodiment of the present invention, the comparative light-emitting device 1, and the comparative light-emitting device 2, light emission originating from [Ir(iPrFptz)₃] that is a blue phosphorescent dopant is obtained. Furthermore, FIG. 27 shows that the light-emitting device 1 and the light-emitting device 2 of one embodiment of the present invention have high external quantum efficiency. That is, CzPIPt and DBTPIPt-II of one embodiment of the present invention were each found to be suitable for a host material of a light-emitting layer in a blue phosphorescent element, and CzPIPt was found to be particularly preferable. Therefore, CzPIPt and DBTPIPt-II were each found to be an organic compound with a high T1 level that can be used as a host of a blue phosphorescent material. FIG. 26 shows that the light-emitting device 1 and the light-emitting device 2 of one embodiment of the present invention can be driven by sufficiently low driving voltage.

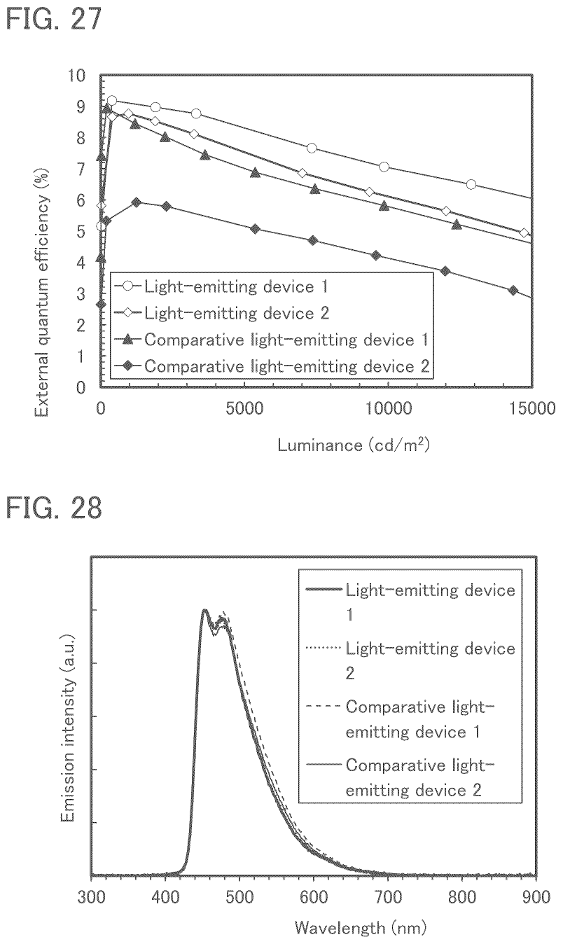

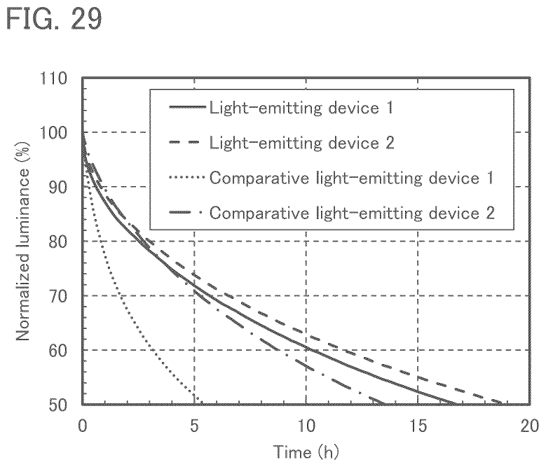

FIG. 29 shows changes in luminance with driving time under the conditions where the initial luminance was 300 cd/m² and the current density was constant. As shown in FIG. 29, the light-emitting device 1 and the light-emitting device 2, which are the light-emitting devices of embodiments of the present invention, were found to be light-emitting devices with a long lifetime. Meanwhile, the comparative light-emitting device 1 exhibiting favorable initial characteristics was found to be a light-emitting device with a short lifetime whose luminance decreases fast.

### Example 5

In this example, a light-emitting device 3 of one embodiment of the present invention and a comparative light-emitting device 3 that is a comparative example are described. Structural formulae of organic compounds used in this example are shown below.

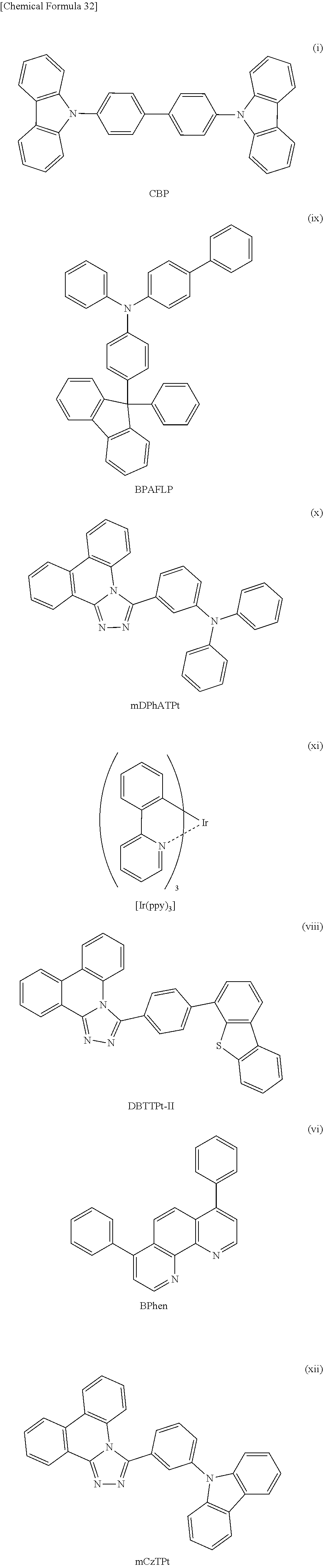

(Method for Fabricating Light-Emitting Device 3)

First, indium tin oxide containing silicon oxide (ITSO) was deposited over a glass substrate by a sputtering method, so that the first electrode 101 was formed. Note that the film thickness was 110 nm and the area of the electrode was 2 mm×2 mm.

Next, in pretreatment for forming the light-emitting device over the substrate, a surface of the substrate was washed with water and baked at 200° C. for 1 hour, and then UV ozone treatment was performed for 370 seconds.

After that, the substrate was transferred into a vacuum evaporation apparatus in which the pressure was reduced to about 1×10⁻⁴ Pa, vacuum baking at 170° C. for 30 minutes was performed in a heating chamber in the vacuum evaporation apparatus, and then the substrate was naturally cooled down for about 30 minutes.

Next, the substrate over which the first electrode 101 was formed was fixed to a substrate holder provided in the vacuum evaporation apparatus so that the surface over which the first electrode 101 was formed faced downward, and 4,4′-di(carbazol-9-yl)biphenyl (abbreviation: CBP) represented by Structural Formula (i) above and molybdenum oxide were co-evaporated over the first electrode 101 to have a weight ratio of 4:2 (=CBP: molybdenum oxide) to a thickness of 60 nm by an evaporation method using resistance heating, whereby the hole-injection layer 111 was formed.

Then, 4-phenyl-4′-(9-phenylfluoren-9-yl)triphenylamine (abbreviation: BPAFLP) represented by Structural Formula (ix) above was deposited by evaporation to a thickness of 30 nm over the hole-injection layer 111 to form the hole-transport layer 112.

Next, 3-(1,2,4-triazolo[4,3-f]phenanthridin-3-yl)triphenylamine (abbreviation: mDPhATPt) represented by Structural Formula (x) above and tris(2-phenylpyridinato-N,C2′)iridium(III) (abbreviation: [Ir(ppy)₃]) represented by Structural Formula (xi) above were co-evaporated to have a weight ratio of 1:0.08 (=mDPhATPt: [Ir(ppy)₃]) to a thickness of 30 nm, whereby the light-emitting layer 113 was formed.

After that, over the light-emitting layer 113, 3-[4-(dibenzothiophen-4-yl)phenyl]-1,2,4-triazolo[4,3-f]phenanthridine (abbreviation: DBTTPt-II) represented by Structural Formula (viii) above was deposited by evaporation to a thickness of 15 nm to form a first electron-transport layer, and then bathophenanthroline (abbreviation: Bphen) represented by Structural Formula (vii) above was deposited by evaporation to a thickness of 15 nm to form a second electron-transport layer; whereby the light-emitting layer 114 was formed.

After the formation of the electron-transport layer 114, lithium fluoride (LiF) was deposited by evaporation to a thickness of 1 nm to form the electron-injection layer 115, and then aluminum was deposited by evaporation to a thickness of 200 nm to form the second electrode 102, whereby a light-emitting device 3 of this example was fabricated.

(Method for Fabricating Comparative Light-Emitting Device 3)

The comparative light-emitting device 3 was fabricated in a manner similar to that of the light-emitting device 3 except that 3-[3-(9H-carbazol-9-yl)phenyl]-1,2,4-triazolo[4,3-f]phenanthridine (abbreviation: mCzTPt) represented by Structural Formula (xii) above was used instead of mDPhATPt in the light-emitting device 3.

The stacked-layer structures of the light-emitting device 3 and the comparative light-emitting device 3 are listed in the following table.

TABLE 3

| | | | Film |
|---|---|---|---|
| | Constituent | Weight | thickness |
| Functional layer name | material | ratio | (nm) |

Light-emitting device 3

| Electron-injection layer | | LiF | — | 1 |
|---|---|---|---|---|
| Electron-transport | 2 | BPhen | — | 15 |
| layer | 1 | DBTTPt-II | — | 15 |

| | | | |
|---|---|---|---|
| Light-emitting layer | mDPhATPt:[Ir(ppy)₃] | 1:0.08 | 30 |
| Hole-transport layer | BPAFLP | — | 30 |
| Hole-injection layer | CBP:MoOx | 4:2 | 60 |

Comparative light-emitting device 3

| Electron-injection layer | | LiF | — | 1 |
|---|---|---|---|---|
| Electron-transport | 2 | BPhen | — | 15 |
| layer | 1 | DBTTPt-II | — | 15 |

| | | | |
|---|---|---|---|
| Light-emitting layer | mCzTPt:[Ir(ppy)₃] | 1:0.08 | 30 |
| Hole-transport layer | BPAFLP | — | 30 |
| Hole-injection layer | CBP:MoOx | 4:2 | 60 |

The light-emitting device 3 and the comparative light-emitting device 3 were subjected to sealing with a glass substrate (a sealant was applied to surround the elements, followed by UV treatment and one-hour heat treatment at 80° C. at the time of sealing) in a glove box containing a nitrogen atmosphere so that the light-emitting devices were not exposed to the air. Then, the initial characteristics of the light-emitting devices were measured.

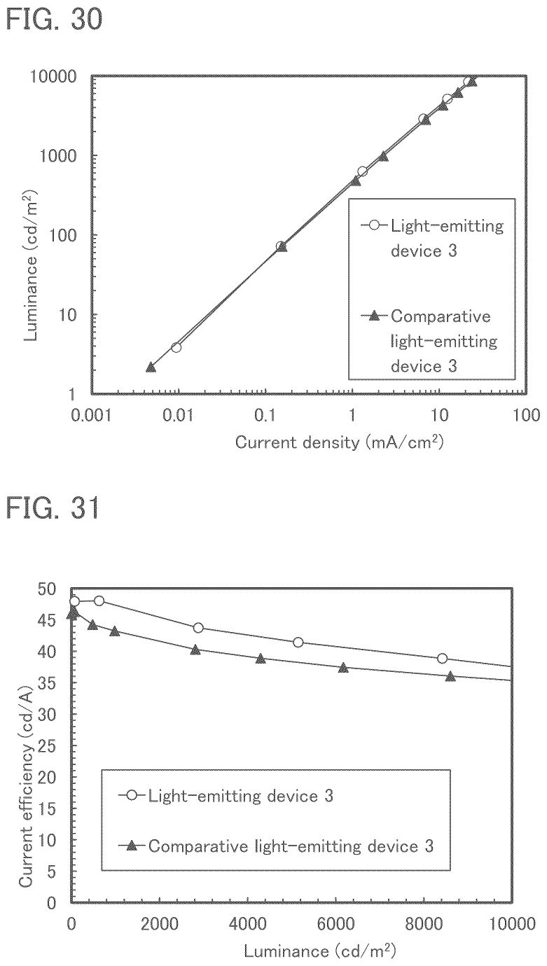

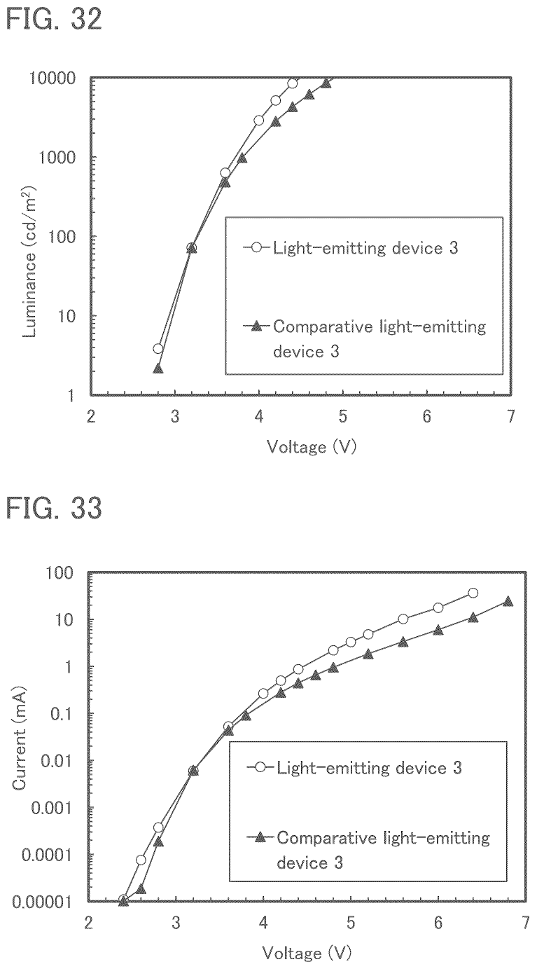

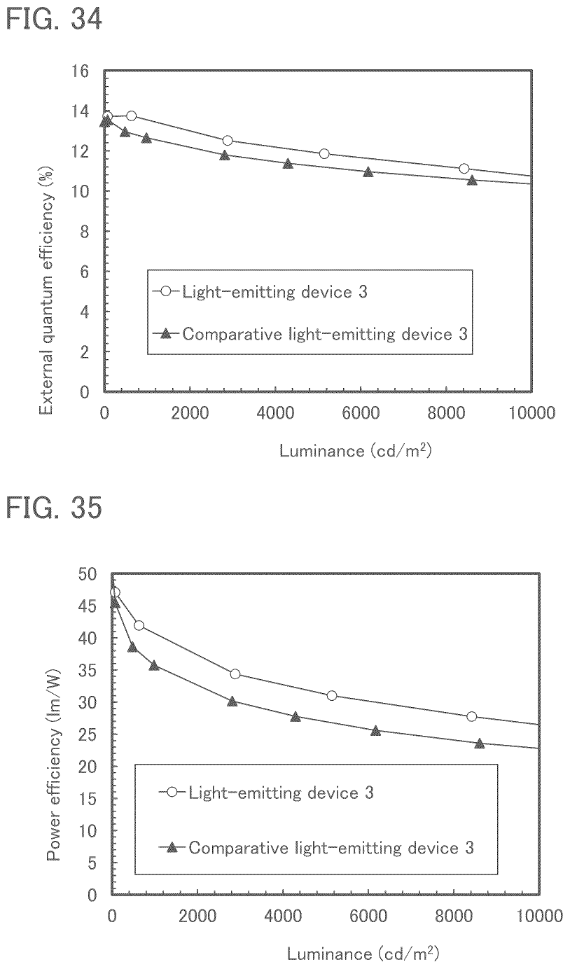

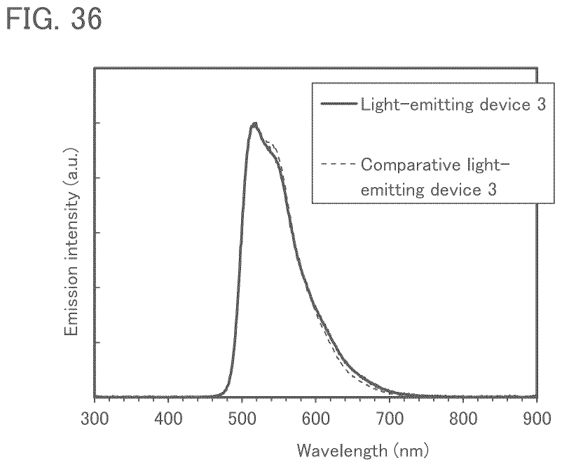

FIG. 30 shows the luminance-current density characteristics of the light-emitting device 1 and the comparative light-emitting device 3; FIG. 31 shows the current efficiency-luminance characteristics thereof; FIG. 32 shows the luminance-voltage characteristics thereof; FIG. 33 shows the current-voltage characteristics thereof; FIG. 34 shows the external quantum efficiency-luminance characteristics thereof; and FIG. 35 shows the luminance-power efficiency characteristics thereof; FIG. 36 shows the emission spectra thereof. Table 4 shows the main characteristics of the light-emitting device 3 and the comparative light-emitting device 3 at approximately 1000 cd/m². Luminance and CIE chromaticity were measured with a luminance colorimeter (BM-5A manufactured by TOPCON TECHNOHOUSE CORPORATION), and electroluminescence spectra were measured with a multi-channel spectrometer (PMA-11 manufactured by Hamamatsu Photonics K.K.). Note that the measurements of the light-emitting devices were performed at room temperature (in an atmosphere maintained at 23° C.).

TABLE 4

| | | | | | | | | External |
|---|---|---|---|---|---|---|---|---|
| | | | Current | | | Current | Power | quantum |
| | Voltage | Current | density | Chromaticity | Chromaticity | efficiency | efficiency | efficiency |
| | (V) | (mA) | (mA/cm²) | y | y | (cd/A) | (lm/W) | (%) |
| Light-emitting device 3 | 3.6 | 0.05 | 1.3 | 0.329 | 0.625 | 48.0 | 41.9 | 13.7 |
| Comparative | 3.8 | 0.09 | 2.3 | 0.333 | 0.621 | 43.2 | 35.7 | 12.7 |
| light-emitting device 3 | | | | | | | | |

FIG. 36 shows that from the light-emitting device 3 of one embodiment of the present invention and the comparative light-emitting device 3, light emission originating from [Ir(ppy)₃] that is a green phosphorescent dopant is obtained. Furthermore, FIG. 31, FIG. 34, and FIG. 35 show that the light-emitting device 3 of one embodiment of the present invention is an EL device having higher current efficiency, higher external quantum efficiency, and higher power efficiency than the comparative light-emitting device 3. FIG. 33 shows that the light-emitting device 3 of one embodiment of the present invention is a light-emitting device that is driven by low voltage and has low power consumption. According to the above, mDPhATPt of one embodiment of the present invention was found to be suitable for a host material of a light-emitting layer in a green phosphorescent element. Therefore, mDPhATPt was found to be an organic compound with a high T1 level that can be used as a host of a green phosphorescent material.

### Example 6

In this example, a light-emitting device 4 of one embodiment of the present invention and a comparative light-emitting device 4 that is a comparative example are described. Structural formulae of organic compounds used in this example are shown below.

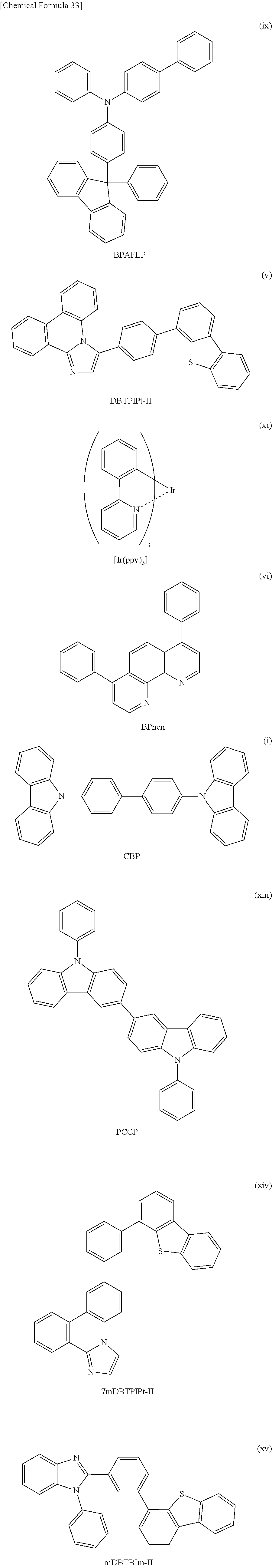

(Method for Fabricating Light-Emitting Device 4)

First, indium tin oxide containing silicon oxide (ITSO) was deposited over a glass substrate by a sputtering method, so that the first electrode 101 was formed. Note that the film thickness was 110 nm and the area of the electrode was 2 mm×2 mm.

Next, in pretreatment for forming the light-emitting device over the substrate, a surface of the substrate was washed with water and baked at 200° C. for 1 hour, and then UV ozone treatment was performed for 370 seconds.

After that, the substrate was transferred into a vacuum evaporation apparatus in which the pressure was reduced to about 1×10⁻⁴ Pa, vacuum baking at 170° C. for 30 minutes was performed in a heating chamber in the vacuum evaporation apparatus, and then the substrate was naturally cooled down for about 30 minutes.

Next, the substrate over which the first electrode 101 was formed was fixed to a substrate holder provided in the vacuum evaporation apparatus so that the surface over which the first electrode 101 was formed faced downward, and 4-phenyl-4′-(9-phenylfluoren-9-yl)triphenylamine (abbreviation: BPAFLP) represented by Structural Formula (ix) above and molybdenum oxide were co-evaporated over the first electrode 101 to have a weight ratio of 4:2 (=BPAFLP: molybdenum oxide) to a thickness of 50 nm by an evaporation method using resistance heating, whereby the hole-injection layer 111 was formed.

Next, over the hole-injection layer 111, BPAFLP was deposited by evaporation to a thickness of 20 nm to form the hole-transport layer 112.

Next, 3-[4-(dibenzothiophen-4-yl)phenyl]imidazo[1,2-f]phenanthridine (abbreviation: DBTPIPt-II) represented by Structural Formula (v) above and tris(2-phenylpyridinato-N,C2′)iridium(III) (abbreviation: [Ir(ppy)₃]) represented by Structural Formula (xi) above were co-evaporated to have a weight ratio of 1:0.06 (=DBTPIPt-II: [Ir(ppy)₃]) to a thickness of 30 nm, whereby the light-emitting layer 113 was formed.

After that, over the light-emitting layer 113, DBTPIPt-II was deposited by evaporation to a thickness of 30 nm to form a first electron-transport layer, and then bathophenanthroline (abbreviation: Bphen) represented by Structural Formula (vi) above was deposited by evaporation to a thickness of 15 nm to form a second electron-transport layer, whereby the electron-transport layer 114 was formed.

After the formation of the electron-transport layer 114, lithium fluoride (LiF) was deposited by evaporation to a thickness of 1 nm to form the electron-injection layer 115, and then aluminum was deposited by evaporation to a thickness of 200 nm to form the second electrode 102, whereby the light-emitting device 4 of this example was fabricated.

(Method for Fabricating Comparative Light-Emitting Device 4)

The comparative light-emitting device 4 is a light-emitting device with the same phosphorescent dopant as that of the light-emitting device 4. The comparative light-emitting device 4 was fabricated in such a manner: in the light-emitting device 4, 4,4-di(N-carbazolyl)biphenyl (abbreviation: CBP) represented by Structural Formula (i) above was used instead of BPAFLP in the hole-injection layer; 3,3′-bis(9-phenyl-9H-carbazole) (abbreviation: PCCP) represented by Structural Formula (xiii) above was used instead of BPAFLP in the hole-transport layer; 7-[3-(dibenzothiophen-4-yl)phenyl]imidazo[1,2-f]phenanthridine (abbreviation: 7mDBTPIPt-II) represented by Structural Formula (xiv) above was used instead of DBTPIPt-II in the light-emitting layer; and N-phenyl-2-[3-(dibenzothiophen-4-yl)phenyl]benzimidazole (abbreviation: mDBTBIm-II) represented by Structural Formula (xv) above was used instead of DBTPIPt-II in the electron-transport layer. Furthermore, the thicknesses of the hole-injection layer 111, the light-emitting layer 113, and the second electron-transport layer are respectively 60 nm, 40 nm, and 20 nm. The materials and thicknesses in the comparative light-emitting device 4 other than the above are the same as those in the light-emitting device 4.

The stacked-layer structures of the light-emitting device 4 and the comparative light-emitting device 4 are listed in the following table.

TABLE 5

| | | | Film |
|---|---|---|---|
| | Constituent | Weight | thickness |
| Functional layer name | material | ratio | (nm) |

Light-emitting device 4

| Electron-injection layer | | LiF | — | 1 |
|---|---|---|---|---|
| Electron-transport | 2 | BPhen | — | 15 |
| layer | 1 | DBTPIPt-II | — | 15 |

| | | | |
|---|---|---|---|
| Light-emitting layer | DBTPIPt-II:[Ir(ppy)₃] | 1:0.06 | 30 |
| Hole-transport layer | BPAFLP | — | 20 |
| Hole-injection layer | BPAFLP:MoOx | 4:2 | 50 |

Comparative light-emitting device 4

| Electron-injection layer | | LIF | — | 1 |
|---|---|---|---|---|
| Electron-transport | 2 | BPhen | — | 20 |
| layer | 1 | mDBTBIm-II | — | 15 |

| | | | |
|---|---|---|---|
| Light-emitting layer | 7mDBTPIPt-II:[Ir(ppy)₃] | 1:0.06 | 40 |
| Hole-transport layer | PCCP | — | 20 |
| Hole-injection layer | CBP:MoOx | 4:2 | 60 |

The light-emitting device 4 and the comparative light-emitting device 4 were subjected to sealing with a glass substrate (a sealant was applied to surround the elements, followed by UV treatment and one-hour heat treatment at 80° C. at the time of sealing) in a glove box containing a nitrogen atmosphere so that the light-emitting devices were not exposed to the air. Then, the initial characteristics of the light-emitting devices were measured.

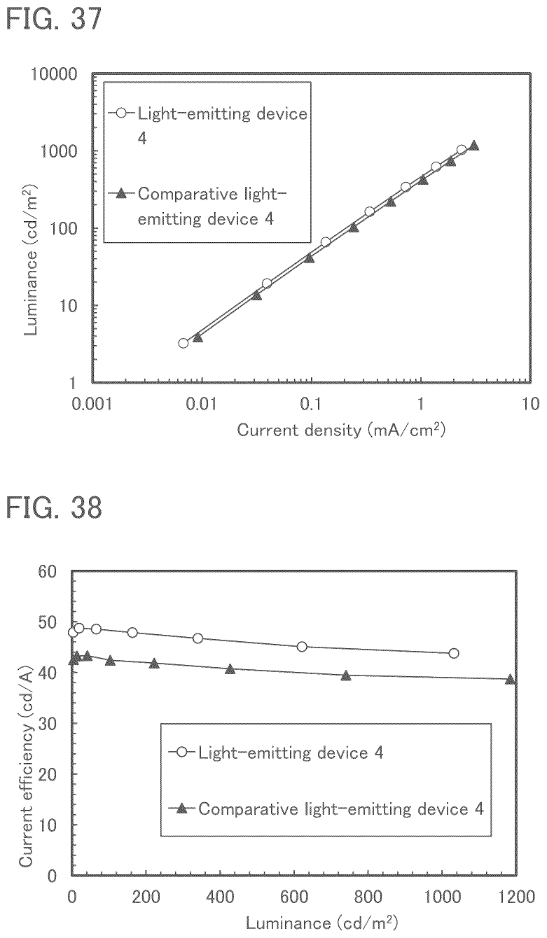

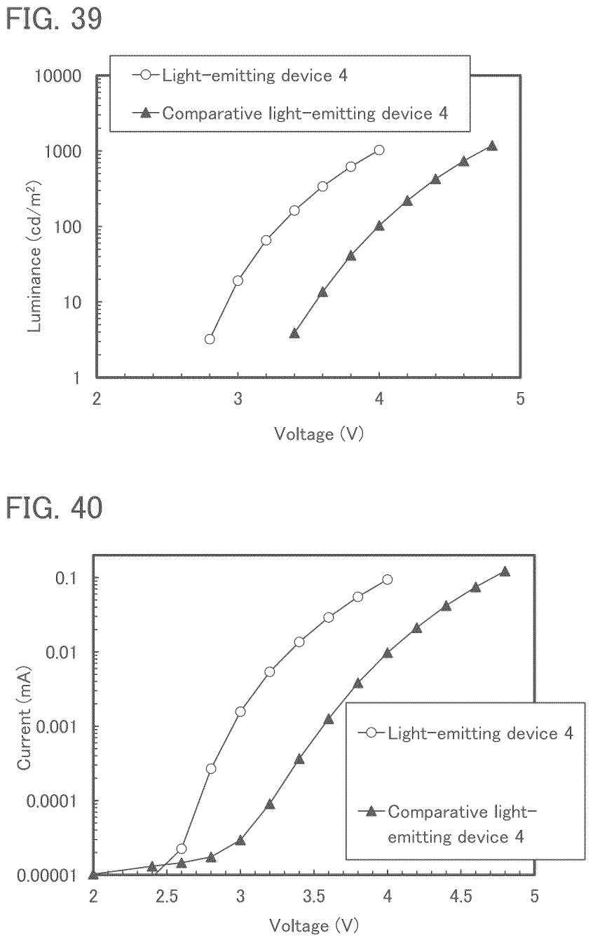

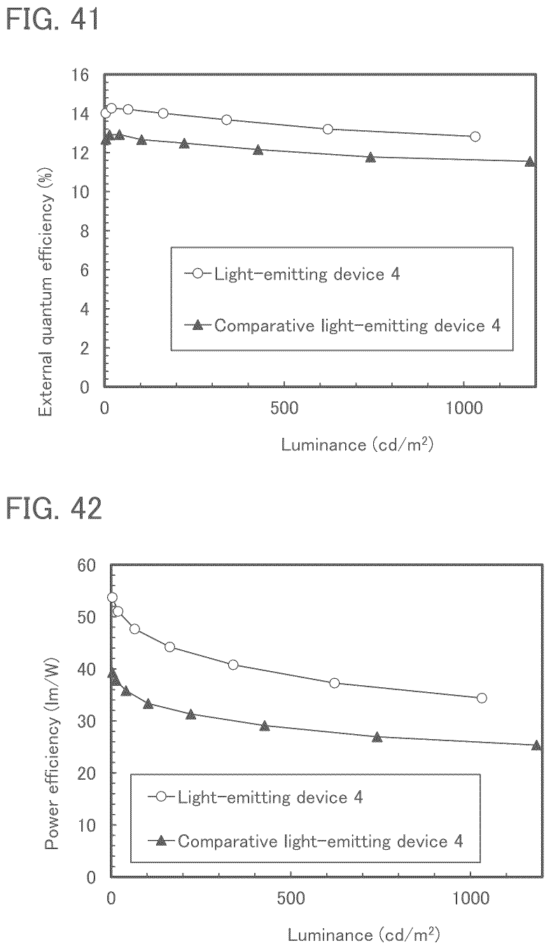

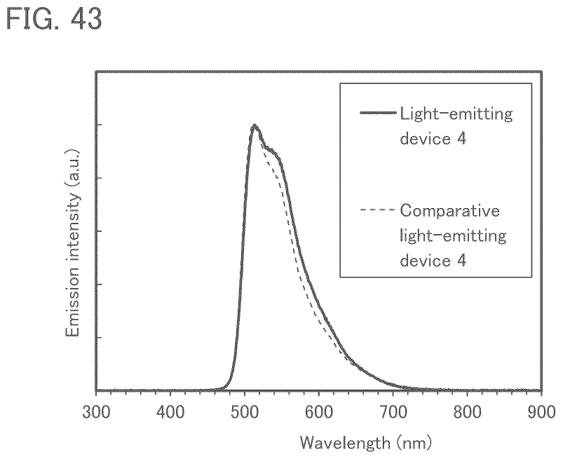

FIG. 37 shows the luminance-current density characteristics of the light-emitting device 4 and the comparative light-emitting device 4; FIG. 38 shows the current efficiency-luminance characteristics thereof; FIG. 39 shows the luminance-voltage characteristics thereof; FIG. 40 shows the current-voltage characteristics thereof; FIG. 41 shows the external quantum efficiency-luminance characteristics thereof; and FIG. 42 shows the luminance-power efficiency characteristics thereof; FIG. 43 shows the emission spectra thereof. Table 6 shows the main characteristics of the light-emitting device 4 and the comparative light-emitting device 4 at approximately 1000 cd/m². Luminance and CIE chromaticity were measured with a luminance colorimeter (BM-5A manufactured by TOPCON TECHNOHOUSE CORPORATION), and electroluminescence spectra were measured with a multi-channel spectrometer (PMA-11 manufactured by Hamamatsu Photonics K.K.). Note that the measurements of the light-emitting devices were performed at room temperature (in an atmosphere maintained at 23° C.).

TABLE 6

| | | | | | | | | External |
|---|---|---|---|---|---|---|---|---|
| | | | Current | | | Current | Power | quantum |
| | Voltage | Current | density | Chromaticity | Chromaticity | efficiency | efficiency | efficiency |
| | (V) | (mA) | (mA/cm²) | x | y | (cd/A) | (lm/W) | (%) |
| Light-emitting device 4 | 4.0 | 0.09 | 2.4 | 0.326 | 0.618 | 43.8 | 34.4 | 12.8 |
| Comparative | 4.8 | 0.12 | 3.1 | 0.307 | 0.621 | 38.7 | 25.3 | 11.6 |
| light-emitting device 4 | | | | | | | | |

FIG. 43 shows that from the light-emitting device 4 of one embodiment of the present invention and the comparative light-emitting device 4, light emission originating from [Ir(ppy)₃] that is a green phosphorescent dopant is obtained. Furthermore, FIG. 38, FIG. 41, and FIG. 42 show that the light-emitting device 4 of one embodiment of the present invention is an EL device having higher current efficiency, higher external quantum efficiency, and higher power efficiency than the comparative light-emitting device 4. FIG. 40 shows that the light-emitting device 4 of one embodiment of the present invention is a light-emitting device that is driven by low voltage and has low power consumption. According to the above, DBTPIPt-II of one embodiment of the present invention was found to be suitable for a host material of a light-emitting layer in a green phosphorescent element. Therefore, DBTPIPt-II was found to be an organic compound with a high T1 level that can be used as a host of a green phosphorescent material. This indicates that a phosphorescent dopant with a higher T1 level and a shorter wavelength can be excited and higher efficiency can be achieved when a substituent is put to the 3-position of a 4-(dibenzothiophen-4-yl)phenyl]imidazo[1,2-f]phenanthridine skeleton as in DBTPIPt-II than when a substituent is put to the 7-position as in 7mDBTPIPt-II. Furthermore, it is indicated that DBTPIPt-II is preferably used for an electron-transport layer because the electron-transport property can be high and the driving voltage can be low.

### Example 7

In this example, a light-emitting device 5 of one embodiment of the present invention described in the embodiment and a comparative light-emitting device 5 that is a comparative example are described. Structural formulae of organic compounds used in this example are shown below.

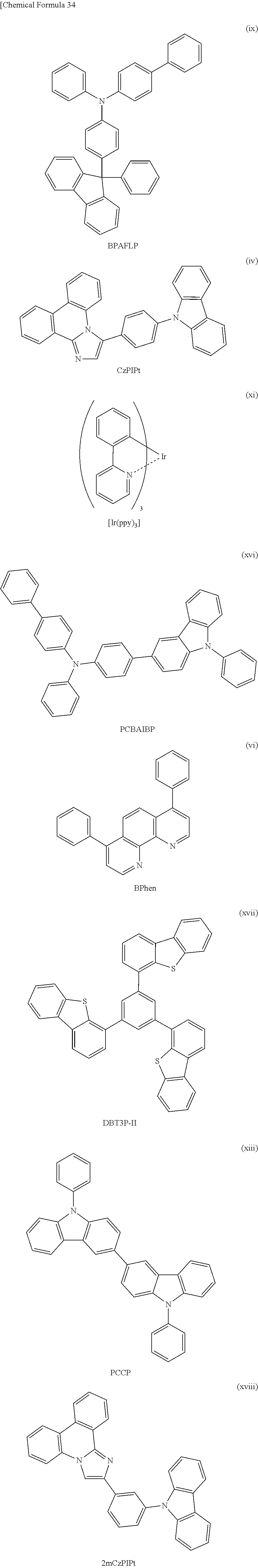

(Method for Fabricating Light-Emitting Device 5)

First, indium tin oxide containing silicon oxide (ITSO) was deposited over a glass substrate by a sputtering method, so that the first electrode 101 was formed. Note that the film thickness was 110 nm and the area of the electrode was 2 mm×2 mm.

Next, in pretreatment for forming the light-emitting device over the substrate, a surface of the substrate was washed with water and baked at 200° C. for 1 hour, and then UV ozone treatment was performed for 370 seconds.

After that, the substrate was transferred into a vacuum evaporation apparatus in which the pressure was reduced to about 1×10⁻⁴ Pa, vacuum baking at 170° C. for 30 minutes was performed in a heating chamber in the vacuum evaporation apparatus, and then the substrate was naturally cooled down for about 30 minutes.

Next, the substrate over which the first electrode 101 was formed was fixed to a substrate holder provided in the vacuum evaporation apparatus so that the surface over which the first electrode 101 was formed faced downward, and 4-phenyl-4′-(9-phenylfluoren-9-yl)triphenylamine (abbreviation: BPAFLP) represented by Structural Formula (ix) above and molybdenum oxide were co-evaporated over the first electrode 101 to have a weight ratio of 2:1 (=BPAFLP: molybdenum oxide) to a thickness of 50 nm by an evaporation method using resistance heating, whereby the hole-injection layer 111 was formed.

Next, over the hole-injection layer 111, BPAFLP was deposited by evaporation to a thickness of 20 nm to form the hole-transport layer 112.

Next, 3-[4-(carbazol-9-yl)phenyl]imidazo[1,2-f]phenanthridine (abbreviation: CzPIPt) represented by Structural Formula (iv) above, 4-phenyl-4′-(9-phenyl-9H-carbazol-3-yl)triphenylamine (abbreviation: PCBA1BP) represented by Structural Formula (xvi) above, and tris(2-phenylpyridinato-N,C2′)iridium(III) (abbreviation: [Ir(ppy)₃]) represented by Structural Formula (xi) above were co-evaporated to have a weight ratio of 1:0.3:0.06 (=CzPIPt: PCBA1BP: [Ir(ppy)₃]) to a thickness of 20 nm to form a first light-emitting layer, and then CzPIPt and [Ir(ppy)₃] were co-evaporated to have a weight ratio of 1:0.06 (=CzPIPt: [Ir(ppy)₃]) to form a second light-emitting layer, whereby the light-emitting layer 113 was formed.

After that, over the light-emitting layer 113, CzPIPt was deposited by evaporation to a thickness of 15 nm to form a first electron-transport layer, and then bathophenanthroline (abbreviation: Bphen) represented by Structural Formula (vi) above was deposited by evaporation to a thickness of 15 nm to form a second electron-transport layer, whereby the electron-transport layer 114 was formed.

After the formation of the electron-transport layer 114, lithium fluoride (LiF) was deposited by evaporation to a thickness of 1 nm to form the electron-injection layer 115, and then aluminum was deposited by evaporation to a thickness of 200 nm to form the second electrode 102, whereby the light-emitting device 5 of this example was fabricated.

(Method for Fabricating Comparative Light-Emitting Device 5)

The comparative light-emitting device 5 is a light-emitting device with the same phosphorescent dopant as that of the light-emitting device 5. The comparative light-emitting device 5 was fabricated in such a manner: in the light-emitting device 5, 4,4′,4″-(benzene-1,3,5-triyl)tri(dibenzothiophene) (abbreviation: DBT3P-II) represented by Structural Formula (xvii) above was used instead of BPAFLP in the hole-injection layer; 3,3′-bis(9-phenyl-9H-carbazole) (abbreviation: PCCP) represented by Structural Formula (xiii) above was used instead of BPAFLP used for the hole-transport layer and PCBA1BP used for the first light-emitting layer; 2-[3-(carbazol-9-yl)phenyl]imidazo[1,2-f]phenanthridine (abbreviation: 2mCzPIPt) represented by Structural Formula (xviii) above was used instead of CzPIPt used for the light-emitting layer and the first electron-transport layer. Furthermore, the thicknesses of the hole-injection layer 111 and the first electron-transport layer are respectively 60 nm and 10 nm. The materials and thicknesses in the comparative light-emitting device 5 other than the above are the same as those in the light-emitting device 5.

The stacked-layer structures of the light-emitting device 5 and the comparative light-emitting device 5 are listed in the following table.

TABLE 7

| | | | Film |
|---|---|---|---|
| | Constituent | Weight | thickness |
| Functional layer name | material | ratio | (nm) |

Light-emitting device 5

| Electron-injection layer | | LiF | — | 1 |
|---|---|---|---|---|
| Electron-transport layer | 2 | BPhen | — | 15 |
| | 1 | CzPIPt | — | 15 |
| Light-emitting layer | 2 | CzPIPt:[Ir(ppy)₃] | 1:0.06 | 10 |
| | 1 | CzPIPt:PCBA1BP:[Ir(ppy)₃] | 1:0.3:0.06 | 20 |

| | | | |
|---|---|---|---|
| Hole-transport layer | BPAFLP | — | 20 |
| Hole-injection layer | BPAFLP:MoOx | 4:2 | 50 |

Comparative light-emitting device 5

| Electron-injection layer | | LiF | — | 1 |
|---|---|---|---|---|
| Electron-transport layer | 2 | Bphen | — | 15 |
| | 1 | 2mCzPIPt | — | 10 |
| Light-emitting layer | 2 | 2mCzPIPt:[Ir(ppy)₃] | 1:0.06 | 10 |
| | 1 | 2mCzPIP:PCCP:[Ir(ppy)₃] | 1:0.3:0.06 | 20 |

| | | | |
|---|---|---|---|
| Hole-transport layer | PCCP | — | 20 |
| Hole-injection layer | DBT3P-II:MoOx | 4:2 | 60 |

The light-emitting device 5 and the comparative light-emitting device 5 were subjected to sealing with a glass substrate (a sealant was applied to surround the elements, and at the time of sealing, UV treatment was performed first and heat treatment was performed at 80° C. for one hour) in a glove box containing a nitrogen atmosphere so that the light-emitting devices were not exposed to the air. Then, the initial characteristics were measured.

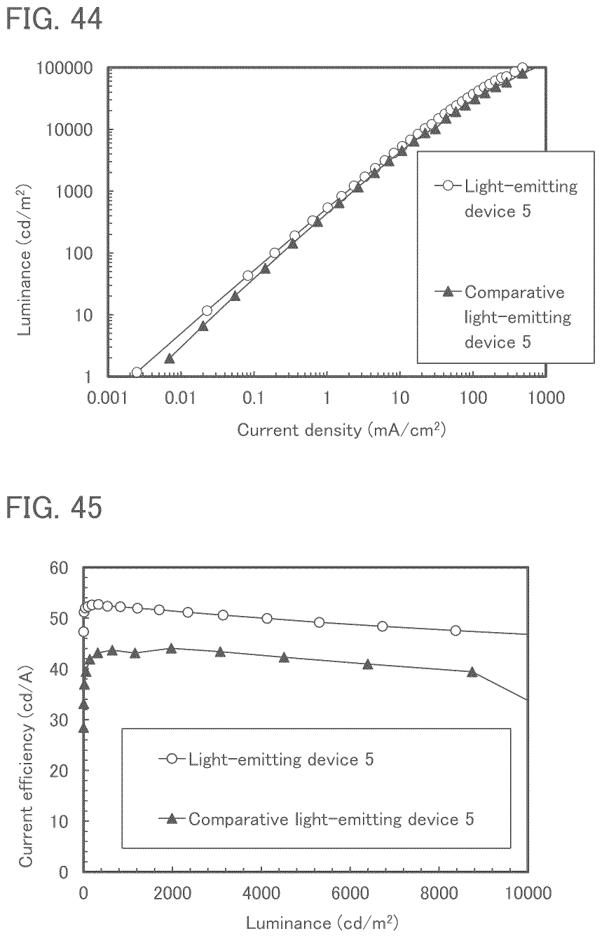

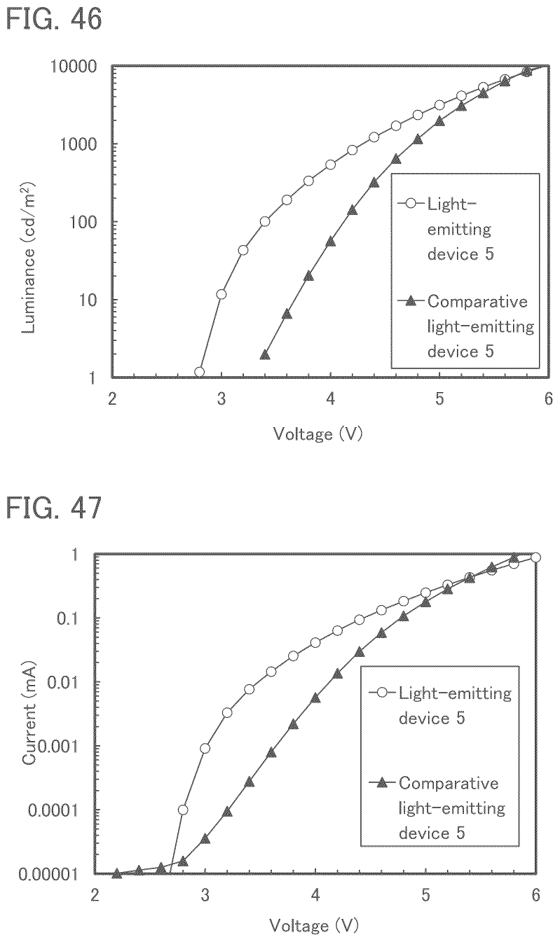

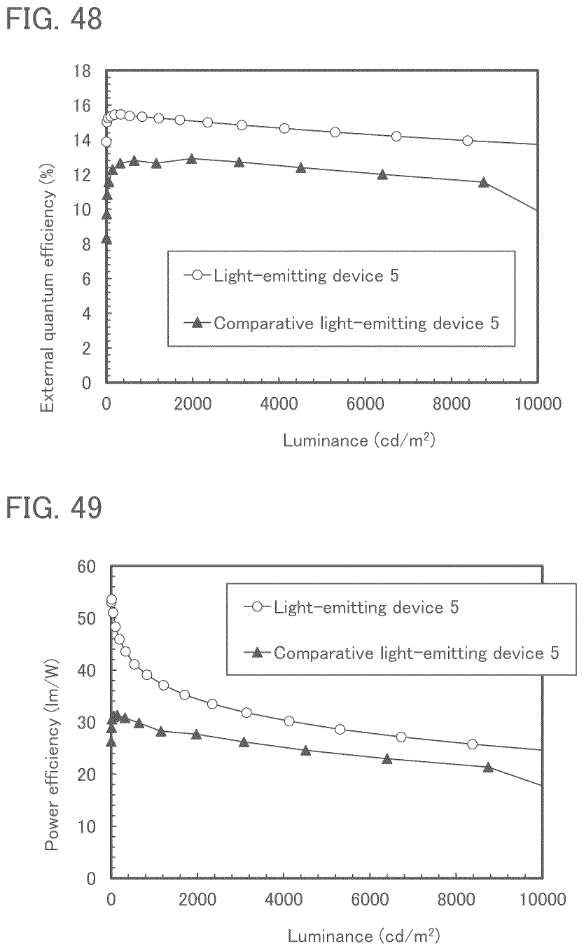

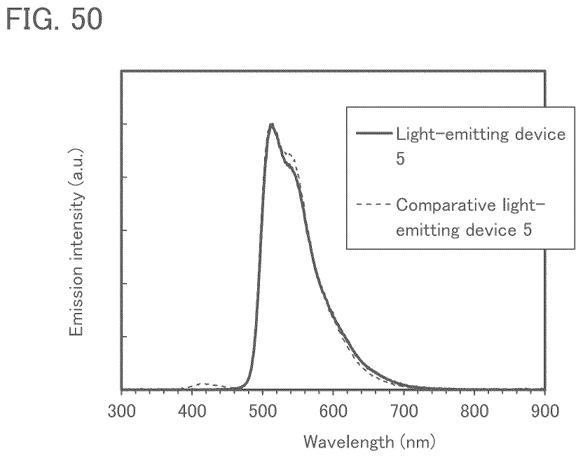

FIG. 44 shows the luminance-current density characteristics of the light-emitting device 5 and the comparative light-emitting device 5; FIG. 45 shows the current efficiency-luminance characteristics thereof; FIG. 46 shows the luminance-voltage characteristics thereof; FIG. 47 shows the current-voltage characteristics thereof; FIG. 48 shows the external quantum efficiency-luminance characteristics thereof; and FIG. 49 shows the luminance-power efficiency characteristics thereof; FIG. 50 shows the emission spectra thereof. Table 8 shows the main characteristics of the light-emitting device 5 and the comparative light-emitting device 5 at approximately 1000 cd/m². Luminance and CIE chromaticity were measured with a luminance colorimeter (BM-5A manufactured by TOPCON TECHNOHOUSE CORPORATION), and electroluminescence spectra were measured with a multi-channel spectrometer (PMA-11 manufactured by Hamamatsu Photonics K.K.). Note that the measurements of the light-emitting devices were performed at room temperature (in an atmosphere maintained at 23° C.).

TABLE 8

| | | | | | | | | External |
|---|---|---|---|---|---|---|---|---|
| | | | Current | | | Current | Power | quantum |
| | Voltage | Current | density | Chromaticity | Chromaticity | efficiency | efficiency | efficiency |
| | (V) | (mA) | (mA/cm²) | x | y | (cd/A) | (lm/W) | (%) |
| Light-emitting device 5 | 4.2 | 0.06 | 1.6 | 0.316 | 0.622 | 52.2 | 39.1 | 15.3 |
| Comparative | 4.6 | 0.06 | 1.5 | 0.307 | 0.608 | 43.7 | 29.8 | 12.8 |
| light-emitting device 5 | | | | | | | | |

FIG. 50 shows that from the light-emitting device 5 of one embodiment of the present invention and the comparative light-emitting device 5, light emission originating from [Ir(ppy)₃] that is a green phosphorescent dopant is obtained. Furthermore, FIG. 45, FIG. 48, and FIG. 49 show that the light-emitting device 5 of one embodiment of the present invention is a light-emitting device having higher current efficiency, higher external quantum efficiency, and higher power efficiency than the comparative light-emitting device 5. FIG. 47 shows that the light-emitting device 5 of one embodiment of the present invention is a light-emitting device that is driven by low voltage and has low power consumption. According to the above, CzPIPt of one embodiment of the present invention was found to be suitable for a host material of a light-emitting layer in a green phosphorescent element. Therefore, CzPIPt was found to be an organic compound with a high T1 level that can be used as a host of a green phosphorescent material. This indicates that a phosphorescent dopant with a higher T1 level and a shorter wavelength can be excited and higher efficiency can be achieved when a substituent is put to the 3-position of a 4-(dibenzothiophen-4-yl)phenyl]imidazo[1,2-f]phenanthridine skeleton as in CzPIPt than when a substituent is put to the 2-position as in 2mCzPIPt. Furthermore, it was found that CzPIPt is preferably used as a material forming an electron-transport layer because the electron-transport property can be high and the driving voltage can be low.

### REFERENCE NUMERALS

101: first electrode, 102: second electrode, 103: EL layer, 111: hole-injection layer, 112: hole-transport layer, 113: light-emitting layer, 114: electron-transport layer, 115: electron-injection layer, 116: charge-generation layer, 117: P-type layer, 118: electron-relay layer, 119: electron-injection buffer layer, 400: substrate, 401: first electrode, 403: EL layer, 404: second electrode, 405: sealant, 406: sealant, 407: sealing substrate, 412: pad, 420: IC chip, 501: anode, 502: cathode, 511: first light-emitting unit, 512: second light-emitting unit, 513: charge-generation layer, 601: driver circuit portion (source line driver circuit), 602: pixel portion, 603: driver circuit portion (gate line driver circuit), 604: sealing substrate, 605: sealant, 607: space, 608: wiring, 609: FPC (flexible print circuit), 610: element substrate, 611: switching FET, 612: current control FET, 613: first electrode, 614: insulator, 616: EL layer, 617: second electrode, 618: light-emitting device, 951: substrate, 952: electrode, 953: insulating layer, 954: partition layer, 955: EL layer, 956: electrode, 1001: substrate, 1002: base insulating film, 1003: gate insulating film, 1006: gate electrode, 1007: gate electrode, 1008: gate electrode, 1020: first interlayer insulating film, 1021: second interlayer insulating film, 1022: electrode, 1024W: first electrode, 1024R: first electrode, 1024G: first electrode, 1024B: first electrode, 1025: partition, 1028: EL layer, 1029: second electrode, 1031: sealing substrate, 1032: sealant, 1033: transparent base material, 1034R: red coloring layer, 1034G: green coloring layer, 1034B: blue coloring layer, 1035: black matrix, 1036: overcoat layer, 1037: third interlayer insulating film, 1040: pixel portion, 1041: driver circuit portion, 1042: peripheral portion, 2001: housing, 2002: light source, 2100: robot, 2110: arithmetic device, 2101: illuminance sensor, 2102: microphone, 2103: upper camera, 2104: speaker, 2105: display, 2106: lower camera, 2107: obstacle sensor, 2108: moving mechanism, 3001: lighting device, 5000: housing, 5001: display portion, 5002: display portion, 5003: speaker, 5004: LED lamp, 5006: connection terminal, 5007: sensor, 5008: microphone, 5012: support, 5013: earphone, 5100: cleaning robot, 5101: display, 5102: camera, 5103: brush, 5104: operation button, 5150: portable information terminal, 5151: housing, 5152: display region, 5153: bend portion, 5120: dust, 5200: display region, 5201: display region, 5202: display region, 5203: display region, 7101: housing, 7103: display portion, 7105: stand, 7107: display portion, 7109: operation key, 7110: remote controller, 7201: main body, 7202: housing, 7203: display portion, 7204: keyboard, 7205: external connection port, 7206: pointing device, 7210: second display portion, 7401: housing, 7402: display portion, 7403: operation button, 7404: external connection port, 7405: speaker, 7406: microphone, 9310: portable information terminal, 9311: display panel, 9313: hinge, 9315: housing

1. An organic compound represented by General Formula (G1), 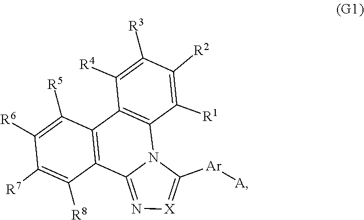
wherein X represents nitrogen or substituted or unsubstituted carbon,
wherein Ar represents a substituted or unsubstituted arylene group having 6 to 12 carbon atoms,
wherein R¹ to R⁸ each independently represent any of hydrogen, an alkyl group having 1 to 6 carbon atoms, a cyclic alkyl group having 3 to 7 carbon atoms, and a substituted or unsubstituted aryl group having 6 to 13 carbon atoms, and
wherein A represents a substituted or unsubstituted diarylamino group when X represents nitrogen, and A represents any of a substituted or unsubstituted diarylamino group, a substituted or unsubstituted carbazolyl group, a substituted or unsubstituted dibenzothiophenyl group, and a substituted or unsubstituted dibenzofuranyl group when X represents carbon.

2. The organic compound according to claim 1,
wherein the R¹ to R⁸ each represent hydrogen.

3. The organic compound according to claim 1, wherein the organic compound is represented by General Formula (G2), 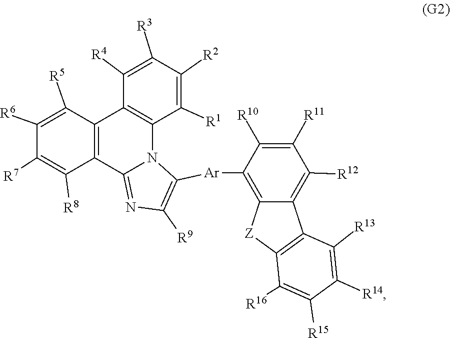
wherein Z represents oxygen or sulfur, and
wherein R⁹ to R¹⁶ each independently represent any of hydrogen, an alkyl group having 1 to 6 carbon atoms, a cyclic alkyl group having 3 to 7 carbon atoms, and a substituted or unsubstituted aryl group having 6 to 13 carbon atoms.

4. The organic compound according to claim 3,
wherein the R¹ to R¹⁶ each represent hydrogen.

5. The organic compound according to claim 1,
wherein the organic compound is represented by General Formula (G3), 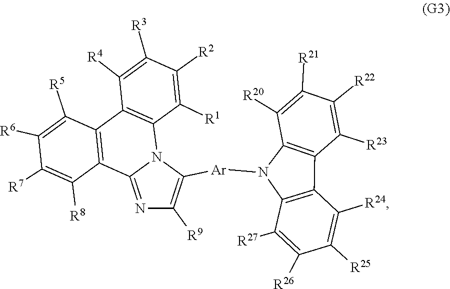
wherein R⁹ and R²⁰ to R²⁷ each independently represent any of hydrogen, an alkyl group having 1 to 6 carbon atoms, a cyclic alkyl group having 3 to 7 carbon atoms, and a substituted or unsubstituted aryl group having 6 to 13 carbon atoms.

6. The organic compound according to claim 5,
wherein the R¹ to R⁹ and R²⁰ to R²⁷ each represent hydrogen.

7. The organic compound according to claim 1,
wherein the Ar is represented by any of Structural Formulae (Ar-1) to (Ar-13),

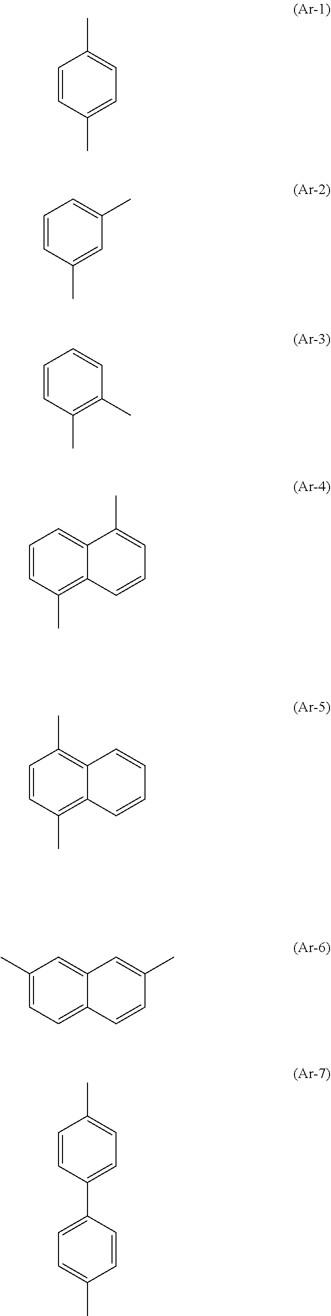

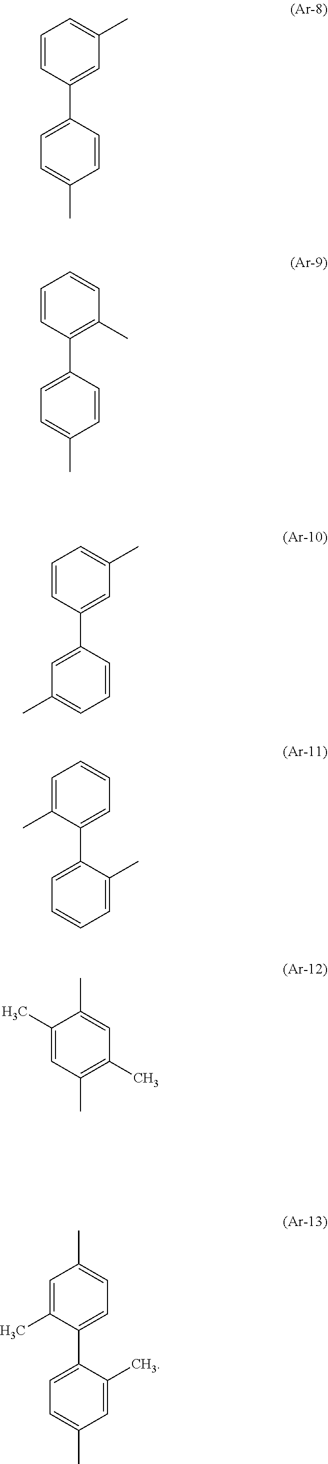

8. The organic compound according to claim 7,
wherein the Ar is (Ar-1).

9. An organic compound represented by Structural Formula (100),

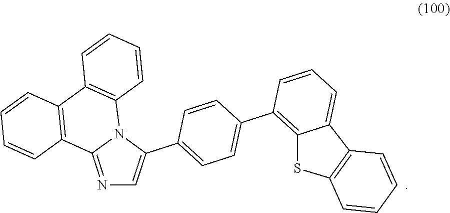 [SMILES: c1ccc2c(c1)sc1c(-c3ccc(-c4cnc5c6ccccc6c6ccccc6n45)cc3)cccc12]

10. An organic compound represented by Structural Formula (135),

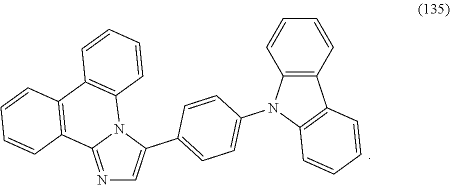 [SMILES: c1ccc2c(c1)c1ccccc1n1c(-c3ccc(-n4c5ccccc5c5ccccc54)cc3)cnc21]

11. A material for a carrier-transport layer of a light-emitting device comprising the organic compound according to claim 1.

12. A host material of a light-emitting device comprising the organic compound according to claim 1.

13. A light-emitting device comprising:
an anode;
a cathode; and
an EL layer positioned between the anode and the cathode,
wherein the EL layer comprises a light-emitting layer, and
wherein the light-emitting layer comprises a light-emitting material and the organic compound according to claim 1.

14. An electronic device comprising:
the light-emitting device according to claim 13, and
a sensor, an operation button, a speaker, or a microphone.

15. A light-emitting apparatus comprising the light-emitting device according to claim 13 and a transistor or a substrate.

16. A lighting device comprising the light-emitting device according to claim 13 and a housing.

17. A material for a carrier-transport layer of a light-emitting device comprising the organic compound according to claim 9.

18. A host material of a light-emitting device comprising the organic compound according to claim 9.

19. A light-emitting device comprising:
an anode;
a cathode; and
an EL layer positioned between the anode and the cathode,
wherein the EL layer comprises a light-emitting layer, and
wherein the light-emitting layer comprises a light-emitting material and the organic compound according to claim 9.

20. An electronic device comprising:
the light-emitting device according to claim 19, and
a sensor, an operation button, a speaker, or a microphone.

21. A light-emitting apparatus comprising the light-emitting device according to claim 19 and a transistor or a substrate.

22. A lighting device comprising the light-emitting device according to claim 19 and a housing.

23. A material for a carrier-transport layer of a light-emitting device comprising the organic compound according to claim 10.

24. A host material of a light-emitting device comprising the organic compound according to claim 10.

25. A light-emitting device comprising:
an anode;
a cathode; and
an EL layer positioned between the anode and the cathode,
wherein the EL layer comprises a light-emitting layer, and
wherein the light-emitting layer comprises a light-emitting material and the organic compound according to claim 10.

26. An electronic device comprising:
the light-emitting device according to claim 25, and
a sensor, an operation button, a speaker, or a microphone.

27. A light-emitting apparatus comprising the light-emitting device according to claim 25 and a transistor or a substrate.

28. A lighting device comprising the light-emitting device according to claim 25 and a housing.
